(19) United States
(12) Patent Application Publication
(10) Pub. No.: US 2005/0026929 A1
(43) Pub. Date: Feb. 3, 2005

(54) Novel phenyl derivatives as inducers of apoptosis

(75) Inventors: Sui Xiong Cai, San Diego, CA (US); Ben Cebon, Kew (AU); Joane Litvak, Oakland, CA (US); Keith Pararajasingham, So. San Francisco, CA (US); Emma J. Shelton, Menlo Park, CA (US); Jeffrey R. Spencer, So. San Francisco, CA (US); David Sperandio, Mountain View, CA (US); Paul A. Sprengeler, El Granada, CA (US); Vincent W.-F. Tai, So. San Francisco, CA (US); Robert M. Yee, San Francisco, CA (US)

Correspondence Address: AXYS PHARMACEUTICALS, INC., 180 KIMBALL WAY, SOUTH SAN FRANCISCO, CA, 94080, US

(73) Assignees: AXYS PHARMACEUTICALS, INC., So. San Francisco, CA (US); CYTOVIA, INC., San Diego, CA (US)

(21) Appl. No.: 10/421,372

(22) Filed: Apr. 23, 2003

Related U.S. Application Data
(60) Provisional application No. 60/374,872, filed on Apr. 23, 2002.

(51) Int. Cl. C07D 417/02; C07D 413/02; C 07D 4 3/02; A61K 31/496; A61K 31/454; A61K 31/422; A61K 31/427

(52) U.S. Cl. 514/254.02; 514/326; 514/365; 514/374; 514/383; 514/396; 544/369; 544/370; 546/208; 546/209

(57) ABSTRACT

The present invention related to certain phenyl derivatives that are activators of caspases and inducers of apoptosis, pharmaceutical composition comprising these compounds and method of treating cancer utilizing these compounds.

### CROSS-REFERENCE

The Applicants claim priority under 35 U.S.C. 119(e) to copending Provisional Application No. 60/374,872 filed on Apr. 23, 2002, the disclosure of which is incorporated herein by reference in its entirety.

### BACKGROUND OF THE INVENTION

1. Field of the Invention

The present invention relates to certain phenyl derivatives that are activators of caspases and inducers of apoptosis, pharmaceutical composition comprising these compounds and method of treating cancer utilizing these compounds. Methods of preparing these compounds are also disclosed.

2. State of the Art

Organisms eliminate unwanted cells by a process variously known as regulated cell death, programmed cell death or apoptosis. Such cell death occurs as a normal aspect of animal development as well as in tissue homeostasis and aging (Glucksmann, A., Biol. Rev. Cambridge Philos. Soc. 26:59-86 (1951); Glucksmann, A., Archives de Biologie 76:419-437 (1965); Ellis, et al., Dev. 112:591-603 (1991); Vaux, et al. Cell 76:777-779 (1994)). Apoptosis regulates cell number, facilitates morphogenesis, removes harmful or otherwise abnormal cells and eliminates cells that have already performed their function. Additionally, apoptosis occurs in response to various physiological stresses, such as hypoxia or ischemia (PCT published application WO96/20721).

There are a number of morphological changes shared by cells experiencing regulated cell death, including plasma and nuclear membrane blebbing, cell shrinkage (condensation of nucleoplasm and cytoplasm), organelle relocalization and compaction, chromatin condensation and production of apoptotic bodies (membrane enclosed particles containing intracellular material) (Orrenius, S., J Internal Medicine 237:529-536 (1995).

Apoptosis is achieved through an endogenous mechanism of cellular suicide (Wyllie, A. H., in Cell Death in Biology and Pathology, Bowen and Lockshin, eds., Chapman and Hall (1991), pp. 9-34). A cell activates its internally encoded suicide program as a result of either internal or external signals. The suicide program is executed through the activation of a carefully regulated genetic program (Wyllie, et al., Int Rev. Cyt. 68:251 (1980); Ellis, et al., Ann Rev. Cell Bio. 7:663 (1991). Apoptotic cells and bodies are usually recognized and cleared by neighboring cells or macrophages before lysis. Because of this clearance mechanism, inflammation is not induced despite the clearance of great numbers of cells (Orrenius, S., J. Internal Medicine 237:529-536(1995)).

A group of proteases are a key element in apoptosis (see, e.g., Thorneberry, Chemistry and Biology 5:R97-R103 (1998); Thornberry, British Med. Bull. 53:478-490 (1996)). Genetic studies in the nematode Caenorhabditis elegans revealed that apoptotic cell death involves at least 14 genes, two of which are the pro-apoptotic (death-promoting) ced (for cell death abnormal) genes, ced-3 and ced-4. CED-3 is homologous to interleukin 1 beta-converting enzyme, a cysteine protease, which is now called caspase-1. Further extensive research revealed that the mammalian apoptosis system appears to involve a cascade of caspases, or a system that behaves like a cascade of caspases. At present, the caspase family of cysteine proteases comprises 14 different members, and more may be discovered in the future. All known caspases are synthesized as zymogens that require cleavage at an aspartyl residue prior to forming the active enzyme. Thus, caspases are capable of activating other caspases in the manner of an amplifying cascade.

Apoptosis and caspases are thought to be crucial in the development of cancer (Apoptosis and Cancer Chemotherapy, Hickman and Dive, eds., Humana Press (1999)). There is mounting evidence that cancer cells, while containing caspases, lack parts of the molecular machinery that activate the caspase cascade. This makes the cancer cells lose their capacity to undergo cellular suicide and the cells become immortal, i.e., they become cancerous. Control points are known to exist in the apoptosis process that represent points for intervention leading to activation. These control points include the CED-9-BCL-like and CED-3-ICE-like gene family products, which are intrinsic proteins regulating the fate of a cell to survive or die, respectively, and executing part of the cell death process itself (see, Schmitt, et al., Biochem. Cell. Biol. 75:301-314 (1997)). BCL-like proteins include BCL-XL and BAX-alpha, which appear to function upstream of caspase activation. BCL-XL appears to prevent activation of the apoptotic protease cascade, whereas BAX-alpha accelerates activation of the apoptotic protease cascade.

Chemotherapeutic (anti-cancer) drugs can trigger cancer cells to undergo suicide by activation of the dormant caspase cascade. This may be a crucial aspect of the mode of action of most, if not all, known anticancer drugs (Los, et al., Blood 90:3118-3129 (1997); Friesen, et al., Nat. Med. 2:574 (1996)). The mechanism of action of current antineoplastic drugs frequently involves an attack at specific phases of the cell cycle. The cell cycle refers to the stages through which cells normally progress during their lifetimes. Normally, cells exist in a resting phase termed G₀. During multiplication, cells progress to a stage in which DNA synthesis occurs, termed S. Later, cell division, or mitosis, occurs in a phase called M. Antineoplastic drugs such as cytosine arabinoside, hydroxyurea, 6-mercaptopurine, and methotrexate are S phase specific, whereas antineoplastic drugs such as vincristine, vinblastine, and paclitaxel are M phase specific. Many slow growing tumors, for example colon cancers, exist primarily in the G₀ phase, whereas rapidly proliferating normal tissues, for example bone marrow, exist primarily in the S or M phase. Thus, the possibility exists for the activation of the caspase cascade, although the exact mechanisms for doing so presently are not clear. Furthermore, insufficient activity of the caspase cascade and consequent apoptotic events are implicated in various types of cancer. The development of caspase cascade activators and inducers of apoptosis is a highly desirable goal in the development of therapeutically effective antineoplastic agents. Moreover, since autoimmune disease and certain degenerative diseases also involve the proliferation of abnormal cells, therapeutic treatment for these diseases could be effected by enhancement of the apoptotic process through the administration of appropriate caspase cascade activators and inducers of apoptosis.

### SUMMARY OF THE INVENTION

In one aspect, this invention is directed to a compound of Formula I: 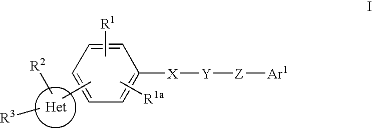
wherein:
R¹ and R^{1a} are independently hydrogen, alkyl, alkoxy, halo, haloalkyl, nitro, amino, alkylamino, dialkylamino, alkylcarbonylamino, carboxy, alkoxycarbonyl, carboxyalkyl, alkoxycarbonylalkenyl, hydroxyalkyl, carboxyalkenyl, hydroxy, alkoxycarbonylalkyloxy, alkoxycarbonylalkyl, carboxyalkylcarbonylamino, or saturated or unsaturated heterocycloalkylaminocarbonylalkyl; or when R¹ and R^{1a} are adjacent to each other they may combine to form a —CH═CH—CH═CH— group;
R² is hydrogen, alkyl, hydroxyalkyl, aryl, heteroaryl, or halo;
R³ is —CONR¹R⁵ where R⁴ and R together with the nitrogen atom to which they are attached form saturated or unsaturated heterocycloalkylamino, saturated or unsaturated bicyclic heterocycloalkylamino, or saturated or unsaturated bridged heterocycloalkylamino;
Het is a five membered heteroaryl ring consisting of one, two, three, or four heteroatoms independently selected from nitrogen, oxygen, or sulfur, the remaining ring atoms being carbon;
X is alkylene optionally substituted with halo;
Y is —O—, —NR⁶—, —SO—, —SO—, —SO₂—, —NR⁷CO—, —CONR⁷—, —NR⁷SO₂—, —SO₂NR⁷—, —NHCONH—, —NHCSNH—, —NHCOO—, or —OCONH— where R⁶ and R⁷ are independently hydrogen or alkyl;
Z is alkenylene or alkylene wherein said alkylene is optionally substituted with halo, hydroxy, hydroxyalkyl, carboxy, amino, amido, alkoxycarbonyl, alkylaminocarbonyl, or dialkylaminocarbonyl; and
Ar¹ is aryl, heteroaryl, or saturated or unsaturated heterocycloalkyl; or a pharmaceutically acceptable salt thereof, provided that:
(i) when Het is oxazol-2-yl, R¹, R^{1a} and R² are hydrogen, X and Z are independently methylene, Y is —NHCO—, and Ar¹ is 4-methoxyphenyl, thien-2-yl, or 2,5-dimethoxyphenyl then R³ is not piperidin-1-yl, 4-methylpiperidin-1-yl, 4-phenylpiperazin-1-yl, 4-(2-methoxyphenyl)piperazin-1-yl, 4-methylpiperazin-1-yl, 4-acetylpiperazin-1-yl, or -3,4-methylenedioxybenzyl; and
(ii) when Het is oxazol-2-yl, R¹, R^{1a}, and R² are hydrogen, X is methylene, Y is —NHCO—, Z is ethylene, and Ar¹ is phenyl then R³ is not piperidin-1-yl, 4-methylpiperidin-1-yl, 4-phenylpiperazin-1-yl, 4-(2-methoxyphenyl)piperazin-1-yl, 4-methylpiperazin-1-yl, 4-acetylpiperazin-1-yl, or 3,4-methylenedioxybenzyl.

Preferably a compound of Formula I, as represented by Ia: 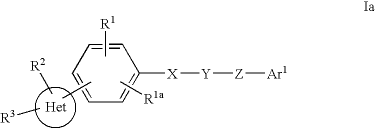
wherein:
R¹, R^{1a}, R², R³, Het, X, Y, and Z are as defined in Formula I above and
Ar¹ is aryl, heteroaryl, or saturated or unsaturated heterocycloalkyl; or a pharmaceutically acceptable salt thereof, provided that (i) when Het is oxazol-2-yl, R¹, R^{1a}, and R² are hydrogen, X and Z are independently alkylene, Y is —NHCO—, and Ar¹ is thien-2-yl or phenyl substituted with alkoxy, then R³ is not piperidin-1-ylcarbonyl optionally substituted with alkyl or piperazin-1-ylcarbonyl optionally substituted with alkyl, alkylcarbonyl, phenyl, 2-methoxyphenyl, or 3,4-methylenedioxybenzyl; and (ii) when Het is oxazol-2-yl, R¹, R^{1a}, and R² are hydrogen, X is alkylene, Z is ethylene, Y is —NHCO—, and Ar¹ is phenyl, then R³ is not piperidin-1-ylcarbonyl optionally substituted with alkyl or piperazin-1-ylcarbonyl optionally substituted with alkyl, alkylcarbonyl, phenyl, methoxyphenyl, or 3,4-methylenedioxybenzyl.

Preferably a compound of Formula I, as represented by Ib: 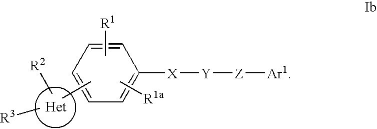
wherein:
R¹ and R^{1a} are independently hydrogen, alkyl, alkoxy, halo, haloalkyl, nitro, amino, alkylamino, dialkylamino, acylamino, or hydroxyalkyl; or when R¹ and R^{1a} are adjacent to each other they may combine to form a CH═CH—CH═CH— group;
R² is hydrogen, alkyl, hydroxyalkyl, aryl, heteroaryl, or halo;
R³ is —CONR⁴R⁵ where R⁴ and R⁵ together with the nitrogen atom to which they are attached form saturated or unsaturated heterocycloalkylamino;
Het is a five membered heteroaryl ring consisting of one, two, three, or four heteroatoms independently selected from nitrogen, oxygen, or sulfur, the remaining ring atoms being carbon;
X is alkylene optionally substituted with halo;
Y is —O—, —NR⁶—, —S—, —SO—, —SO₂—, —NR⁷CO—, —CONR¹-, —NR⁷SO₂—, —SO₂NR⁷—, —NHCONH—, —NHCSNH—, —NHCOO—, or —OCONH— where R⁶ and R⁷ are independently hydrogen or alkyl;
Z is alkylene optionally substituted with halo or alkenylene; and
Ar¹ is aryl, heteroaryl, or saturated or unsaturated heterocycloalkyl; or a pharmaceutically acceptable salt thereof, provided that:
(i) when Het is oxazol-2-yl, R¹, R^{1a}, and R² are hydrogen, X and Z are independently alkylene, Y is —NHCO—, and Ar¹ is thien-2-yl or phenyl substituted with alkoxy, then R³ is not piperidin-1-ylcarbonyl optionally substituted with alkyl or piperazin-1-ylcarbonyl optionally substituted with alkyl, 2-methoxyphenyl, or 3,4-methylenedioxybenzyl; and
(ii) when Het is oxazol-2-yl, R¹, R^{1a}, and R² are hydrogen, X is alkylene, Z is ethylene, Y is —NHCO—, and Ar¹ is phenyl, then R³ is not piperidin-1-ylcarbonyl optionally substituted with alkyl or piperazin-1-ylcarbonyl optionally substituted with alkyl, methoxyphenyl, or 3,4-methylenedioxybenzyl.

In a second aspect, this invention is directed to a method of treating a disorder responsive to the induction of apoptosis in an animal suffering said disorder, comprising administering to said animal a pharmaceutical composition comprising a therapeutically effective amount of a compound of Formula II: 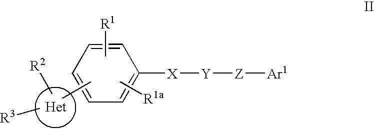
wherein:
R¹ and R^{1a} are independently hydrogen, alkyl, alkoxy, halo, haloalkyl, nitro, amino, alkylamino, dialkylamino, alkylcarbonylamino, carboxy, alkoxycarbonyl, carboxyalkyl, alkoxycarbonylalkenyl, hydroxy, alkoxycarbonylalkyloxy, alkoxycarbonylalkyl, carboxyalkylcarbonylamino, carboxyalkenyl, saturated or unsaturated heterocycloalkylaminocarbonylalkyl, or hydroxyalkyl; or when R¹ and R^{1a} are adjacent to each other they may combine to form a —CH═CH—CH═CH— group;

R² is hydrogen, alkyl, hydroxyalkyl, aryl, heteroaryl, or halo;

R³ is —CONR⁴R⁵ where R⁴ and R⁵ together with the nitrogen atom to which they are attached form saturated or unsaturated heterocycloalkylamino, saturated or unsaturated bicyclic heterocycloalkylamino or bridged saturated or unsaturated heterocycloalkylamino;
Het is a five membered heteroaryl ring consisting of one, two, three, or four heteroatoms independently selected from nitrogen, oxygen, or sulfur, the remaining ring atoms being carbon;
X is alkylene optionally substituted with halo;
Y is —O—, —NR⁶—, —S—, —SO—, —SO₂—, —NR⁷CO—, —CONR⁷—, —NR⁷SO₂—, —SO₂NR⁷—, —NHCONH—, —NHCSNH—, —NHCOO—, or —OCONH— where R⁶ and R⁷ are independently hydrogen or alkyl;
Z is alkenylene or alkylene wherein said alkylene is optionally substituted with halo, hydroxy, hydroxyalkyl, carboxy, amino, amido, alkoxycarbonyl, alkylaminocarbonyl, or dialkylaminocarbonyl; and
Ar¹ is aryl, heteroaryl, or saturated or unsaturated heterocycloalkyl; or a pharmaceutically acceptable salt thereof.

Preferably, a compound of Formula II, as represented by IIa: 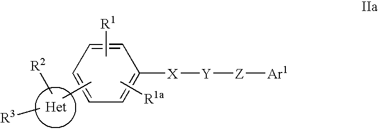
wherein:
R¹ and R^{1a} are independently hydrogen, alkyl, alkoxy, halo, haloalkyl, nitro, amino, alkylamino, dialkylamino, acylamino, or hydroxyalkyl; or when R¹ and R^{1a} are adjacent to each other they may combine to form a CH═CH—CH═CH— group;
R² is hydrogen, alkyl, hydroxyalkyl, aryl, heteroaryl, or halo;
R³ is —CONR⁴R⁵ where R⁴ and R⁵ together with the nitrogen atom to which they are attached form saturated or unsaturated heterocycloalkylamino;
Het is a five membered heteroaryl ring consisting of one, two, three, or four heteroatoms independently selected from nitrogen, oxygen, or sulfur, the remaining ring atoms being carbon;
X is alkylene optionally substituted with halo;
Y is —O—, —NR⁶—, —S—, —SO—, —SO₂—, —NR⁷CO—, —CONR⁷—, —NR⁷SO₂—, —SO₂NR⁷—, —NHCONH—, —NHCSNH—, —NHCOO—, or —OCONH— where R⁶ and R⁷ are independently hydrogen or alkyl;
Z is alkylene optionally substituted with halo or alkenylene; and
Ar¹ is aryl, heteroaryl, or saturated or unsaturated heterocycloalkyl; or a pharmaceutically acceptable salt thereof.

Preferably, the disorder is a cancer, autoimmune disease, rheumatoid arthritis, inflammatory bowel disease, or psoriasis. Preferably, the cancer is selected from the group consisting of Hodgkin's disease, non-Hodgkin's lymphoma, acute and chronic lymphocytic leukemias, multiple myeloma, neuroblastoma, breast carcinoma, ovarian carcinoma, lung carcinoma, Wilms' tumor, cervical carcinoma, testicular carcinoma, soft-tissue sarcoma, chronic lymphocytic leukemia, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head and neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer and prostatic carcinoma, and the animal is a human. More preferably, the cancer is selected from the group consisting of non-Hodgkin's lymphoma, lung carcinoma, testicular carcinoma, chronic lymphocytic leukemia, small-cell lung carcinoma, and colon carcinoma.

In third aspect, this invention is directed to a pharmaceutical composition comprising a therapeutically effective amount of a compound of Formula I, Ia, Ib, or II and a pharmaceutically acceptable excipient.

In a fourth aspect, this invention is directed to a method of treating cancer in an animal which method comprises administering to said animal a pharmaceutical composition comprising a therapeutically effective amount of a compound of Formula I or II and a pharmaceutically acceptable excipient in combination with radiation therapy and optionally in combination with one or more chemotherapeutic compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.

Preferably, the chemotherapeutic compound(s) is independently selected from Taxol®, Taxotere®, epothilone A, epothilone B, desoxyepothilone A, desoxyepothilone B or their derivatives; epidophyllotoxin; procarbazine; mitoxantrone; the mitomycins, discodermolide, podophyllotoxins, doxorubicin, carminomycin, daunorubicin, aminopterin, methotrexate, methopterin, dichloromethotrexate, mitomycin C, porfiromycin, Herceptin®, Rituxan®, 5 fluorouracil, 6-mercaptopurine, gemcitabine, cytosine arabinoside, colchicines, etoposide, etoposide phosphate, teniposide, melphalan, vinblastine, vincristine, vinorelbein, leurosidine, vindesine, leurosine, paclitaxel, estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons, interleukins, capecitabine, and gefitinib. More preferably, the chemotherapeutic compound(s) is independently selected from cisplatin, gemcitabine, 5-fluorouracil, capecitabine, and gefitinib.

In a fifth aspect, this invention is directed to an intermediate of Formula III: 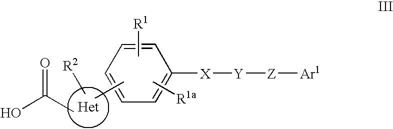
where R¹, R^{1a} R², X, Y, Z, and Ar¹ are as defined above for a compound of Formula I, including preferred embodiments;
or a compound of Formula IV: 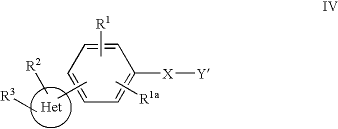
where R¹, R^{1a}, R², R³, X, are as defined above for a compound of Formula I, including preferred embodiments; and Y′ is —OH, —SH, or —NHR″ where R″ is hydrogen or a nitrogen protecting group.

In a sixth aspect, this invention is directed to a process of preparing a compound of Formula I or II where Y is —NR⁷CO— comprising:

(a) reacting a compound of Formula III: 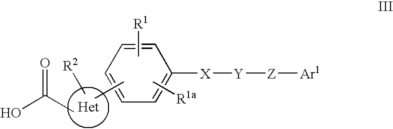
where R¹, R^{1a}, R², X, Z, and Ar¹ are as defined for a compound of Formula I above and Y is —NR⁷CO— where R⁷ is as defined for a compound of Formula I above; with an amine of formula NHR⁴R⁵ where R⁴ and R⁵ together with the nitrogen atom to which they are attached form saturated or unsaturated heterocycloalkylamino, saturated or unsaturated bicyclic heterocycloalkylamino, or bridged saturated or unsaturated heterocycloalkylamino to provide a compound of Formula I or II; or
(b) reacting a compound of Formula IV: 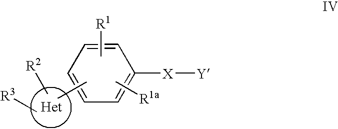
where R¹, R^{1a}, R², R³, X, are as defined for a compound of Formula I above and Y′ is —NHR⁷ where R⁷ is as defined for a compound of Formula I above, with an acylating agent of formula Ar¹—Z—CO₂H or Ar¹—Z—COLG where LG is a leaving group under acylating reaction conditions to provide a compound of Formula I or II, where Y is —NR⁷CO—;
(c) optionally converting the compound obtained in step (a) or (b) above, to an acid addition salt;
(d) optionally converting a salt form of the compound obtained in step (a) or (b) above, to a free base;
(e) optionally separating individual isomers;
(f) optionally modifying any of the R¹, R^{1a}, R², R³, and Ar¹ groups.

### DETAILED DESCRIPTION OF THE INVENTION

Definitions:

Unless otherwise stated, the following terms used in the specification and claims are for the purposes of this Application and have the following meanings: “Acyl” means a radical COR where R is alkyl or trifluoromethyl, e.g., methylcarbonyl, or trifluoromethylcarbonyl, and the like.

“Acylamino” means a radical —NHCOR where R is alkyl or trifluoromethyl, e.g., acetylamino or trifluoromethylcarbonylamino, and the like.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), or pentyl (including all isomeric forms), and the like.

“Alkylene” means a linear saturated divalent hydrocarbon radical of one to six carbon atoms or a branched saturated divalent hydrocarbon radical of three to six carbon atoms e.g., methylene, ethylene, propylene, 1-methylpropylene, 2-methylpropylene, butylene, or pentylene, and the like.

“Alkenyl” means a linear monovalent hydrocarbon radical of two to six carbon atoms or a branched monovalent hydrocarbon radical of three to six carbon atoms containing one or two double bonds e.g., ethenyl, propenyl, 2-propenyl, 1-methylpropenyl, butenyl, or pentenyl, and the like.

“Alkenylene” means a linear divalent hydrocarbon radical of two to six carbon atoms or a branched divalent hydrocarbon radical of three to six carbon atoms containing one or two double bonds e.g., ethenylene, propenylene, 1-methylpropenylene, butenylene, or pentenylene, and the like.

“Alkoxy” means a radical —OR where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, or 2-ethoxyethyl, and the like.

“Alkoxycarbonyl” means a radical —COOR where R is alkyl as defined above, e.g., methoxycarbonyl, ethoxycarbonyl, n-propoxycarbonyl, or 2-propoxycarbonyl, n-, iso-, or tert-butoxycarbonyl, and the like.

“Alkoxycarbonylalkenyl” means a radical-(alkenylene)-COOR, where R is alkyl as defined above, e.g., 2-methoxycarbonyl-1-ethenyl, or 3-ethoxycarbonyl-2-propenyl, and the like.

“Alkoxycarbonylalkyl” means a radical-(alkylene)-COOR, where R is alkyl, as defined above, e.g., methoxycarbonylethyl, and the like.

“Alkoxycarbonylalkyloxy” means a radical —O-(alkylene)-COOR where R is alkyl as defined above, e.g. methoxycarbonylmethyloxy, and the like.

“Alkylamino” means a radical —NHR where R is alkyl as defined above, or an N-oxide derivative, or a protected derivative thereof, e.g., methylamino, ethylamino, n-, iso-propylamino, n-, iso-, tert-butylamino, or methylamino-N-oxide, and the like.

“Alkylaminocarbonyl” means a radical —CONHR where R is an alkyl group as defined above e.g, methylaminocarbonyl or ethylaminocarbonyl, and the like.

“Alkylcarbonyl” means a radical —(CO)R′ where R′ is an alkyl as defined above, e.g., methylcarbonyl, ethylcarbonyl, or 2-propylcarbonyl, and the like.

“Alkylcarbonylamino” means a radical —NR(CO)R′, where R′ is alkyl as defined above and R is hydrogen or alkyl, e.g., methylcarbonylamino or ethylcarbonylamino, and the like.

“Alkylcarboxy” means a radical O(CO)R where R is alkyl as defined above, e.g., methylcarboxy or ethylcarboxy, and the like.

“Alkylthio” means a radical —SR where R is alkyl as defined above, e.g., methylthio, ethylthio, propylthio (including all isomeric forms), or butylthio (including all isomeric forms), and the like.

“Amino” means a radical —NH₂, or an N-oxide derivative, or a protected derivative thereof such as —NH→₂, —NHBoc, or —NHCBz, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R and R′ are independently selected from hydrogen, alkyl, or —COR^{a} where R^{a} is alkyl, or an N-oxide derivative, or a protected derivative thereof e.g., aminomethyl, methylaminoethyl, 2-ethylamino-2-methylethyl, 1,3-diaminopropyl, dimethylaminomethyl, diethylaminoethyl, or acetylaminopropyl, and the like.

“Aminocarbonyl” means a radical —CONH₂, or an N-oxide derivative, or a protected derivative thereof, and the like.

“Aryl” means a monovalent, monocyclic or bicyclic aromatic hydrocarbon radical of 6 to 12 ring atoms e.g., phenyl, naphthyl, or anthracenyl, and the like. The aryl ring may be optionally fused to a saturated or unsaturated heterocycloalkyl ring and optionally substituted on any of the rings with one, two, or three substituents independently selected from the group consisting of alkyl, alkoxy, alkylthio, azido, haloalkyl, haloalkoxy, halo, hydroxy, amino, alkylamino, dialkylamino, nitro, alkylcarbonyl, alkylcarbonylamino, alkoxycarbonyl, alkoxyalkyl, aminoalkyl, aminocarbonyl, alkylaminocarbonyl, dialkylaminocarbonyl, carboxy, cyano, hydroxyalkyl, optionally substituted phenyl, optionally substituted heteroaryl, or when two substituents are adjacent to each other they can combine to form methylenedioxy group or aryl is pentafluorophenyl.

“Carboxyalkenyl” means a radical-(alkenylene)-COOH, e.g., carboxyethenyl, 1-, 2-, or 3-carboxypropenyl, and the like.

“Carboxyalkyl” means a radical-(alkylene)-COOH, e.g., carboxymethyl, carboxyethyl, 1-, 2-, or 3-carboxypropyl, and the like.

“Carboxyalkylcarbonylamino” means a radical —NRCOR′, where R is hydrogen or alkyl, as defined above and R′ is carboxyalkyl as defined above, e.g., 2-carboxyethylcarbonylamino, and the like.

“Cycloalkenyl” means a cyclic unsaturated hydrocarbon radical of three to six carbon atoms, e.g., cyclopropenyl or cyclohexenyl, and the like.

“Cycloalkyl” means a cyclic saturated monovalent hydrocarbon radical of three to six carbon atoms, e.g., cyclopropyl, cyclobutyl, cyclopentyl, or cyclohexyl, and the like.

“Cycloalkylalkyl” means a -(alkylene)-R where R is cycloalkyl as defined above; e.g., cyclopropylmethyl, cyclobutylmethyl, cyclopentylethyl, or cyclohexylmethyl, and the like.

“Cycloalkylcarbonyloxy” means a —O—(CO)R′, where R′ is cycloalkyl, as defined above, e.g., cyclohexanecarbonyloxy, and the like.

“Dialkylamino” means a radical —NRR′ where R and R′ are independently alkyl as defined above, or an N-oxide derivative, or a protected derivative thereof, e.g., dimethylamino, diethylamino, methylpropylamino, methylethylamino, n-, iso-, or tert-butylamino, and the like.

“Dialkylaminocarbonyl” means a radical CONRR′ where R and R′ are independently an alkyl group as defined above e.g, dimethylaminocarbonyl or methylethylaminocarbonyl, and the like.

“Ethylenedioxy” means a radical —O—(CH₂)₂—O—.

“Halo” means fluoro, chloro, bromo, and iodo, preferably fluoro or chloro.

“Haloalkoxy” means a radical —OR where R is haloalkyl as defined above, e.g., trofluoromethoxy or 2,2,2-trifluoroethoxy, and the like.

“Haloalkyl” means alkyl substituted with one or more halogen atoms, preferably one to three halogen atoms, preferably fluorine or chlorine, including those substituted with different halogens, e.g., —CH₂Cl, —CF₃, or —CHF₂, and the like.

“Heteroaralkyl” means a radical (alkylene)-R radical, where R is heteroaryl as defined below, e.g., pyridinylmethyl, furanylmethyl, or chloropyridinylmethyl, and the like.

“Heteroaryl” means a monovalent monocyclic or bicyclic aromatic radical of 5 to 10 ring atoms containing one or more, preferably one, two, or three ring heteroatoms selected from N, O, or S, SO₂, the remaining ring atoms being carbon. More specifically the term heteroaryl includes, but is not limited to, pyridinyl, pyrrolyl, imidazolyl, thienyl, furanyl, indolyl, quinolyl, pyrazinyl, pyrimidinly, pyridazinyl, oxazolyl, isooxazolyl, benzoxazolyl, quinolinyl, isoquinolinyl, benzopyranyl, or thiazolyl, and the derivatives thereof, or N-oxide or a protected derivative thereof. The heteroaryl ring may be optionally substituted with one, two, or three substituents independently selected from the group consisting of alkyl, alkoxy, alkylthio, haloalkyl, haloalkoxy, halo, hydroxy, amino, alkylamino, dialkylamino, nitro, alkylcarbonyl, alkylcarbonylamino, alkoxycarbonyl, alkoxyalkyl, aminoalkyl, aminocarbonyl, alkylaminocarbonyl, dialkylaminocarbonyl, carboxy, cyano, hydroxyalkyl, or optionally substituted phenyl.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, or 1-(hydroxymethyl)-2-hydroxyethyl, and the like.

“Methylenedioxy” means a radical —O—CH₂—O—.

The present invention also includes the prodrugs of compounds of Formula I or II. The term prodrug is intended to represent covalently bonded carriers, which are capable of releasing the active ingredient of Formula I or II when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula I or II include compounds wherein a hydroxy, amidino, guanidino, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula I or II), amides (e.g, trifluoroacetylamino, acetylamino, and the like), and the like. Prodrugs of compounds of Formula I or II are also within the scope of this invention.

The present invention also includes N-oxide derivatives and protected derivatives of compounds of Formula I or II. For example, when compounds of Formula I or II contain an oxidizable nitrogen atom, the nitrogen atom can be converted to an N-oxide by methods well known in the art. Also when compounds of Formula I or II contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I or II can be prepared by methods well known in the art.

A “pharmaceutically acceptable salt” of a compound means a salt that is pharmaceutically acceptable and that possesses the desired pharmacological activity of the parent compound. Such salts include:
acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula I or II can exist as tautomers. All possible tautomers are within the scope of this invention. Additionally, as used herein the terms alkyl includes all the possible isomeric forms of said alkyl group albeit only a few examples are set forth. Furthermore, when the cyclic groups such as aryl, heteroaryl, heterocycloalkyl are substituted, they include all the positional isomers albeit only a few examples are set forth.

“Optional” or “optionally” means that the subsequently described event or circumstance may but need not occur, and that the description includes instances where the event or circumstance occurs and instances in which it does not. For example, “heterocycloalkyl group optionally mono- or di-substituted with an alkyl group” means that the alkyl may but need not be present, and the description includes situations where the heterocycloalkyl group is mono- or disubstituted with an alkyl group and situations where the heterocycloalkyl group is not substituted with the alkyl group.

“Optionally substituted heteroaryl” means a heteroaryl ring as defined above which is optionally substituted with one, two, or three substituents independently selected from alkyl, halo, alkoxy, trifluoromethyl, trifluoromethoxy, amino, alkylamino, dialkylamino, hydroxy, cyano, nitro, aminocarbonyl, hydroxyalkyl, alkoxycarbonyl, or aminoalkyl. More specifically the term optionally substituted heteroaryl includes, but is not limited to, pyridinyl, pyrrolyl, imidazolyl, thienyl, furanyl, indolyl, quinolyl, pyrazinyl, pyrimidinyl, pyridazinyl, oxazolyl, isooxazolyl, benzoxazolyl, quinolinyl, isoquinolinyl, benzopyranyl, and thiazolyl, and the derivatives thereof, or N-oxide or a protected derivative thereof.

“Optionally substituted heteroaralkyl” means a -(alkylene)-R where R is optionally substituted heteroaryl ring as defined above.

“Optionally substituted phenylalkyl” means a radical-(alkylene)-R where R is optionally substituted phenyl as defined above e.g., benzyl, phenylethyl, and the like.

“Optionally substituted phenyl” means a phenyl ring optionally substituted with one, two, or three substituents independently selected from alkyl, halo, alkoxy, alkylthio, trifluoromethyl, trifluoromethoxy, amino, alkylamino, dialkylamino, hydroxy, cyano, nitro, methylenedioxy, aminocarbonyl, hydroxyalkyl, alkoxycarbonyl, aminoalkyl, or carboxy or optionally substituted with five fluorine atoms.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Saturated heterocycloalkyl” means a saturated monovalent cyclic group of 3 to 10 ring atoms in which one, two, or three ring atoms are heteroatoms selected from N, O, or S(O)ₙ, where n is an integer from 0 to 2, the remaining ring atoms being C where one or two carbon atoms can be optionally be replaced by a carbonyl group. More specifically the term heterocycloalkyl includes, but is not limited to, pyrrolidino, piperidino, morpholino, piperazino, tetrahydropyranyl, and thiomorpholino, and the like, and the derivatives thereof and N-oxide or a protected derivative thereof. The heterocycloalkyl ring may be optionally substituted, on any ring, with one, two, or three substituents independently selected from the group consisting of alkyl, alkoxy, alkoxyalkyl, alkylthio, haloalkyl, haloalkoxy, halo, hydroxy, amino, alkylamino, dialkylamino, nitro, alkylcarbonylamino, carboxy, alkoxycarbonyl, aminoalkyl, aminocarbonyl, alkylaminocarbonyl, dialkylaminocarbonyl, cyano, cycloalkyl, cycloalkylalkyl, cycloalkylcarbonyloxy, optionally substituted phenyl, optionally substituted heteroaryl, optionally substituted phenylalkyl, optionally substituted heteroaralkyl, or hydroxyalkyl.

“Saturated heterocycloalkylamino” means a saturated monovalent cyclic group of 3 to 10 ring atoms in which one of the ring atoms is nitrogen and optionally one, two, or three additional ring atoms are independently selected from —(CO)—, —N—, —O—, —S(O)ₙ where n is 0, 1, or 2, the rest of the ring atoms being carbon. The heterocycloalkyamino group may be optionally substituted on any ring with one, two, or three substituents independently selected from the group consisting of alkyl, alkoxy, alkoxyalkyl, alkylthio, haloalkyl, haloalkoxy, halo, hydroxy, hydroxyalkyl, amino, alkylamino, dialkylamino, nitro, alkylcarbonylamino, alkoxycarbonyl, alkylcarbonyl, alkylcarbonyloxy, aminoalkyl, aminocarbonyl, alkylaminocarbonyl, dialkylaminocarbonyl, carboxy, [(CH₃)₃CO(CO)NH][(CH₃)₃CO(CO)CH₂]—CH(CO)NH—, cyano, cycloalkylcarbonyloxy, —SO₂OH, optionally substituted phenyl, optionally substituted heteroaryl, optionally substituted phenylalkyl, optionally substituted heteroaralkyl, hydroxyalkyl or ethylenedioxy. More specifically the term heterocycloalkylamino; includes, but is not limited to, pyrrolidino, piperidino, morpholino, piperazino, homopiperidino, homopiperazino, and the like, and the derivatives thereof and N-oxide or a protected derivative thereof. Additionally, when heterocycloalkylamino contains a keto group, i.e., —(CO)—, the keto can be protected as the ketal, —OCH₂CH₂O—. Such ketal derivative is within the scope of this invention.

“Saturated bicyclic heterocycloalkylamino” means a saturated monovalent heterocycloalkylamino group, as defined above, that is fused with cycloalkyl or heterocycloalkyl, e.g., 3-aza-bicyclo[3.1.0]hexan-3-yl, decahydro-isoquinolin-2-yl, 8-oxa-3-aza-bicyclo[4.2.0]octan-3-yl, or 7-oxa-3-aza-bicyclo[4.2.0]octan-3-yl, and the like.

“Saturated bridged heterocycloalkylamino” means a saturated bridged monovalent cyclic group of 6 to 10 ring atoms in which one of the ring atoms is nitrogen and optionally one, two, or three additional ring atoms are indpendently selected from —(CO)—, —N—, —O—, —S(O)ₙ where n is 0, 1, or 2, the rest of the ring atoms being carbon. Representative examples include, but are not limited to, 3-aza-bicyclo[3.2.2]nonan-3-yl, and the like.

“Saturated or unsaturated heterocycloalkylaminocarbonylalkyl” means a radical —(alkylene)-(CO)—R′, where R′ is saturated or unsaturated heterocycloalkylamino as defined above, e.g., morpholin-4-yl-carbonylethyl or piperazin-1-yl-carbonylethyl, and the like.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

A “therapeutically effective amount” means the amount of a compound of Formula I or II that, when administered to a mammal for treating a disease, is sufficient to effect such treatment for the disease. The “therapeutically effective amount” will vary depending on the compound, the disease and its severity and the age, weight, etc., of the mammal to be treated.

“Unsaturated bicyclic heterocycloalkylamino” means an unsaturated bridged monovalent cyclic group of means a saturated monovalent heterocycloalkylamino group, as defined above, that is fused with cycloalkenyl or unsubstituted aryl, e.g., 1,2,3,4-tetrahydro-isoquinolin-2-yl, and the like.

“Unsaturated bridged heterocycloalkenylamino” means an unsaturated bridged monovalent cyclic group of 6 to 10 ring atoms in which one of the ring atoms is nitrogen and one or two additional ring atoms are optionally selected from —(CO)—, —N—, —O—, —S(O)ₙ where n is 0, 1, or 2, the rest of the ring atoms being carbon. Representative examples include, but are not limited to, e.g., 3-aza-bicyclo[2.2.1]hept-5-en-3-yl, and the like.

“Unsaturated heterocycloalkyl” means a monovalent cyclic group of 3 to 10 ring atoms containing in which one, two, or three ring atoms are heteroatoms selected from N, O, or S(O)ₙ, where n is an integer from 0 to 2, the remaining ring atoms being C and additionally containing one or two double bonds. More specifically the term unsaturated heterocycloalkyl; includes, but is not limited to, dihydropyrroline, tetrahydropyridine, tetrahydroazepine, tetrahydroisoquinoline, and the like, and the derivatives thereof and N-oxide or a protected derivative thereof.

“Unsaturated heterocycloalkylamino” means a monovalent cyclic group of 3 to 10 ring atoms in which one, two, or three ring atoms are heteroatoms selected from N, O, or S(O)n, where n is an integer from 0 to 2 provided that at least one nitrogen atom is present, the remaining ring atoms being C and which additionally contains one or two double bonds. The heterocycloalkylamino group may be optionally substituted with alkyl, halo, alkoxy, or hydroxy. Examples include, but are not limited to, dihydropyrroline, tetrahydropyridine, tetrahydroazepine, tetrahydroisoquinoline, and the like.

### Preferred Embodiments

While the broadest definition of this invention is set forth in the Summary of the Invention, certain compounds of Formula I or II are preferred. For example:

1. One preferred group of compounds of Formula I or II is that wherein:

Het is selected from the group consisting of oxazol-2-yl, thiazol-2-yl, 1H-imidazol-2-yl, [1,2,4]oxadiazol-3-yl, and 1H-pyrazol-1-yl, preferably oxazol-2-yl; and is located in the 4-position of the phenylene ring, with the carbon to which —X—Y—Z— is attached being in the 1-position;

R² is hydrogen, alkyl, or halo, preferably hydrogen or methyl, more preferably hydrogen; and

Y is —NR⁷SO₂— or —NR⁷CO—, preferably —NHSO₂—, —N(CH₃)CO—, or —NHCO—, more preferably —NHCO—.

Within the above preferred and more preferred groups, an even more preferred group of compounds is that wherein:

R¹ is hydrogen or halo, preferably hydrogen or fluoro; and

R^{1a} is hydrogen, halo, hydroxy, nitro, alkyl, alkoxy, alkoxycarbonylalkenyl, alkoxycarbonylalkyl, carboxyalkenyl, carboxy, alkylcarbonylamino, carboxyalkyl, carboxyalkylcarbonylamino, alkoxycarbonyl, alkoxycarbonylalkyloxy, saturated heterocycloalkylaminocarbonylalkyl, or amino; preferably, R^{1a} is hydrogen, fluoro, iodo, hydroxy, nitro, methyl, methoxy, methoxycarbonylethylen-1-yl, methoxycarbonylethyl, carboxyethylen-1-yl, acetylamino, carboxy, carboxyethyl, 2-carboxyethylcarbonylamino, methoxycarbonyl, methoxycarbonylmethyloxy, 2-(piperazin-1-ylcarbonyl)ethyl, 2-(morpholin-4-ylcarbonyl)ethyl, or amino. More preferably, R^{1a} is hydrogen, 3-fluoro, 5-fluoro, 2-iodo, 2-hydroxy, 3-hydroxy, 2-nitro, 3-methyl, 2-methoxy, 3-methoxy, 2-methoxycarbonylethylen-1-yl, 2-methoxycarbonylethyl, 2-(carboxyethylen-1-yl), 2-acetylamino, 2-carboxy, 2-carboxyethyl, 2-(2-carboxyethylcarbonylamino)-, 2-methoxycarbonyl, 3-(methoxy-carbonylmethyloxy), 2-[2-(piperazin-1-ylcarbonyl)ethyl], 2-[2-(morpholin-4-ylcarbonyl)ethyl], or 2-amino.

Even more preferably, R¹ and R^{1a} are both fluoro or both hydrogen or R¹ is hydrogen and R^{1a} is methyl. Most preferably, R¹ and R^{1a} are both hydrogen or both fluoro where the fluoro are in the three and five positions of the phenylene ring or R¹ is hydrogen and R^{1a} is methyl where the methyl is in the three position of the phenylene ring. Most preferably, R¹ and R^{1a} are both hydrogen or both fluoro where the fluoro are in the three and five positions of the phenylene ring.

Within the above preferred and more preferred groups, a particularly preferred group of compounds is that wherein:

X is methylene or ethylene; preferably, methylene; and

Z is alkylene which is optionally substituted with hydrogen, halo, hydroxy, hydroxyalkyl, carboxy, amino, alkoxycarbonyl, alkylaminocarbonyl, or dialkylaminocarbonyl; more preferably Z is —CH(CH₃)CH₂—, —CH₂—CH(CH₃)—, dimethylmethylene, methylene, ethylene, or propylene wherein methylene, ethylene, or propylene is optionally substituted with hydrogen, fluoro, hydroxy, difluoro, carboxy, amino, hydroxymethyl, ethoxycarbonyl, methylaminocarbonyl, or dimethylaminocarbonyl. Even more preferably, Z is methylene, ethylene, propylene, fluoromethylene, difluoromethylene, hydroxymethylene, S-hydroxymethylene, R-hydroxymethylene, aminomethylene, S-aminomethylene, carboxymethylene, hydroxymethylmethylene, ethoxycarbonylmethylene, methylaminocarbonylmethylene, or dimethylaminocarbonylmethylene. Most preferably, Z is methylene, fluoromethylene, or difluoromethylene. Within any of the above preferred, more preferred, even more preferred, and most preferred groups that contain a chiral center, the stereochemistry may be R or S or a mixture of R and S.

Within the above preferred, more preferred and particularly preferred groups, a more particularly preferred group of compounds is that wherein Ar¹ is phenyl optionally substituted with one or two or three subsitutents independently selected from alkyl, halo, alkoxy, methylenedioxy, azido, haloalkyl, hydroxy, or haloalkoxy; preferably phenyl optionally substituted with one, two, or three substituents independently selected from methyl, chloro, fluoro, iodo, methoxy, methylenedioxy, trifluoromethyl, azido, hydroxy, or trifluoromethoxy. More preferably, Ar¹ is phenyl, 2-methylphenyl, 3-methylphenyl, 4-methylphenyl, 2-methoxyphenyl, 3-methoxyphenyl, 4-methoxyphenyl, 2-fluorophenyl, 3-fluorophenyl, 4-fluorophenyl, 3-chlorophenyl, 4-chlorophenyl, 2,6-dichlorophenyl, 3,4-dichlorophenyl, 2,4-dichlorophenyl, 4-trifluoromethoxyphenyl, 3,4-dimethoxyphenyl, 2,5-dimethoxyphenyl, 4-ethoxy-phenyl, 3,5-dimethylphenyl, 3,4-difluorophenyl, 2,5-bis-(trifluoromethyl)phenyl, 3,4-methylenedioxyphenyl, 4-methoxy-3-methylphenyl, 3,4,5-trimethoxyphenyl, 3-azidophenyl, 4-azidophenyl, 4-iodophenyl, 2-hydroxyphenyl, 3-hydroxyphenyl, 4-hydroxyphenyl, or 3-fluoro-4-hydroxyphenyl. Even more preferably Ar¹ is phenyl, 4-trifluoromethoxyphenyl, 4-chlorophenyl, or 2-fluorophenyl.

Within the above preferred, more and even more preferred, and particularly preferred groups, another more particularly preferred group of compounds is that wherein Ar¹ is heteroaryl, preferably pyridinyl, thienyl, 3-methyl-isoxazol-5-yl, or furanyl; more preferably thien-2-yl, thien-3-yl, pyridin-2-yl, pyridin-3-yl, 3-methyl-isoxazol-5-yl, or furan-2-yl; even more preferably thien-3-yl or thien-2-yl.

Within the above preferred, more preferred, even more preferred groups, particularly and more particularly preferred groups, a more preferred group of compounds is that wherein R³ is —CONR⁴R⁵ where R⁴ and R⁵ together with the nitrogen atom to which they are attached form saturated heterocycloalkylamino, preferably: either
(a) piperidin-1-yl optionally substituted with one, two, or three groups independently selected from hydrogen, alkyl, halo, hydroxy, alkoxy, alkoxycarbonyl, carboxy, haloalkyl, alkylcarbonyloxy, {[(CH₃)₃C]O(CO)NH}{[(CH₃)₃C]O(CO)CH₂}CH(CO)NH, —OSO₂OH, cycloalkylcarbonyloxy, or hydroxyalkyl; preferably, hydrogen, fluoro, bromo, chloro, hydroxy, methyl, methoxy, methoxycarbonyl, ethoxycarbonyl, carboxy, methylcarbonyloxy, —OSO₂OH, hydroxymethyl, trifluoromethyl, {[(CH₃)₃C]O(CO)NH}{[(CH₃)₃C]O(CO)CH₂}CH(CO)NH, hydroxyethyl, or cyclohexylcarbonyloxy; more preferably, piperidin-1-yl, 3,3-difluoropiperidin-1-yl, 2-hydroxymethylpiperidin-1-yl, 3-hydroxymethylpiperidin-1-yl, 3-hydroxypiperidin-1-yl, 3R-hydroxypiperidin-1-yl, 4-hydroxy-piperidin-1-yl, 3,3-difluoro-4-hydroxypiperidin-1-yl, 2-methylpiperidin-1-yl, 3-methylpiperidin-1-yl, 4-methylpiperidin-1-yl, 4-hydroxymethylpiperidin-1-yl, 4-bromopiperidin-1-yl, 2,6-dimethylpiperidin-1-yl, 3,3-dimethylpiperidin-1-yl, 3-methoxypiperidin-1-yl, 4-methoxypiperidin-1-yl, 3-fluoropiperidin-1-yl, 4-fluoropiperidin-1-yl, 4,4-difluoropiperidin-1-yl, 4-chloropiperidin-1-yl, 3-chloropiperidin-1-yl, 4-methoxycarbonylpiperidin-1-yl, 4-carboxypiperidin-1-yl, 4-methylcarbonyloxypiperidin-1-yl, 2-hydroxyethylpiperidin-1-yl, 4-ethoxycarbonylpiperidin-1-yl, 2-methoxycarbonylpiperidin-1-yl, 3,4-dihydroxy-piperidin-1-yl, cis-3,4-dihydroxypiperidin-1-yl, trans-3,4-dihydroxypiperidin-1-yl, 3,5-dimethylpiperidin-1-yl, 3,4-difluoropiperidin-1-yl, 3-trifluoromethylpiperidin-1-yl, 4-trifluoromethylpiperidin-1-yl, 3-fluoro-4-hydroxypiperidin-1-yl, 4-cyclohexylcarbonyloxypiperidin-1-yl, 4-(HOSO₂O—)-3-hydroxypiperidin-1-yl, 4-acetyloxypiperidin-1-yl, cis-3-hydroxy-4-hydroxymethylpiperidin-1-yl, trans-3-hydroxy-4-hydroxymethylpiperidin-1-yl, cis-4-hydroxy-3-hydroxymethylpiperidin-1-yl, or 3-({[(CH₃)₃C]O(CO)NH}{[(CH₃)₃C]O(CO)CH₂}CH(CO)NH)-4-hydroxypiperidin-1-yl; even more preferably, piperidin-1-yl, 3,3-difluoropiperidin-1-yl, 4-hydroxypiperidin-1-yl, 3-hydroxypiperidin-1-yl, 3,3-difluoro-4-hydroxypiperidin-1-yl; or
(b) pyrrolidin-1-yl optionally substituted with hydrogen, alkyl, hydroxy, haloalkyl, alkoxycarbonyl, or hydroxyalkyl; preferably hydrogen, methyl, hydroxy, trifluoromethyl, methoxycarbonyl, or hydroxymethyl; more preferably pyrrolidin-1-yl, 2-methylpyrrolidin-1-yl, 3-hydroxypyrrolidin-1-yl, 2,5-dimethylpyrrolidin-1-yl, cis-2,5-dimethylpyrrolidin-1-yl, trans-2,5-dimethylpyrrolidin-1-yl, 2S-methoxycarbonylpyrrolidin-1-yl, 2S-hydroxymethylpyrrolidin-1-yl, 2R-hydroxymethylpyrrolidin-1-yl, 2-trifluoromethylpyrrolidin-1-yl; or
(c) homopiperidin-1-yl optionally substituted with hydrogen, hydroxy, or halo; preferably, hydrogen, hydroxy, or fluoro; more preferably homopiperidin-1-yl, 3-hydroxyhomopiperidin-1-yl, 4-hydroxyhomopiperidin-1-yl, 4-fluoro-3-hydroxyhomopiperidin-1-yl, trans-4-fluoro-3-hydroxyhomopiperidin-1-yl, or cis-4-fluoro-3-hydroxyhomopiperidin-1-yl; even more preferably homopiperidin-1-yl or 4-hydroxyhomopiperidin-1-yl; or
(d) thiomorpholin-4-yl; or
(e) morpholin-4-yl; or
(f) [1,3]oxazinan-3-yl; or
(g) azetidin-1-yl; or
(h) thiazolidin-3-yl, optionally substituted with hydrogen or alkyl; preferably, hydrogen or methyl; more preferably, thiazolidin-3-yl or 2-methylthiazolidin-3-yl; or
(i) piperazin-1-yl, optionally substituted with hydrogen, alkyl, or alkylcarbonyl; preferably, hydrogen, methyl or methylcarbonyl; more preferably, piperazin-1-yl, 4-acetylpiperazin-1-yl, 2,5-dimethylpiperazin-1-yl, cis-2,5-dimethylpiperazin-1-yl, or trans-2,5-dimethylpiperazin-1-yl; or
(j) homopiperazin-1-yl, optionally substituted with hydrogen, alkyl, or alkylcarbonyl; preferably hydrogen, methyl or acetyl; more preferably, homopiperazin-1-yl, 4-methylpiperazin-lyl or 4-acetylpiperazin-1-yl; or
(k) azocan-1-yl; or
(l) 2-methylaziridin-1-yl; or
(m) [1,4]oxazepan-4-yl; or
(n) piperidin-1-yl where one carbon is replaced by —O—, —SO—, or —SO₂—; preferably, 1-oxothiomorpholin-4-yl, 1,1-dioxothiomorpholin-4-yl, 4-oxopiperidin-1-yl, 3-oxopiperidin-1-yl, or 3-fluoro-4-oxopiperidin-1-yl; or
(O) 3-oxo-piperazin-1-yl.

More preferably, R³ is CONR⁴R⁴ where R⁴ and R⁵ together with the nitrogen atom to which they are attached form 3,3-difluoropiperidin-1-yl, piperidin-1-yl, 4-hydroxypiperidin-1-yl, 3-hydroxypiperidin-1-yl, homopiperidin-1-yl, 4-hydroxyhomopiperidin-1-yl, or 3,3-difluoro-4-hydroxypiperidin-1-yl.

Within the above preferred, more preferred, even more preferred groups, particularly and more particularly preferred groups, a more preferred group of compounds is that wherein R³ is —CONR⁴R⁵ where R⁴ and R⁵ together with the nitrogen atom to which they are attached form unsaturated heterocycloalkylamino, preferably unsaturated heterocycloalkylamino optionally substituted with one, two, or three substituents selected from hydrogen or alkyl; preferably hydrogen or methyl; more preferably, 1,2,3,6-tetrahydro-pyridin-1-yl, 2,5-dimethyl-2,5-dihydro-1H-pyrrol-1-yl, cis-2,5-dimethyl-2,5-dihydro-1H-pyrrol-1-yl, or trans-2,5-dimethyl-2,5-dihydro-1H-pyrrol-1-yl.

Within the above preferred, more preferred, even more preferred groups, particularly and more particularly preferred groups, a more preferred group of compounds is that wherein R³ is —CONR⁴R⁵ where R⁴ and R⁵ together with the nitrogen atom to which they are attached form saturated bicyclic heterocycloalkylamino, preferably decahydro-isoquinolin-2-yl, 3-aza-bicyclo[3.1.0]hexan-3-yl, 8-oxa-3-aza-bicyclo[4.2.0]octan-3-yl, or 7-oxa-3-aza-bicyclo[4.2.0]octan-3-yl.

Within the above preferred, more preferred, even more preferred groups, particularly and more particularly preferred groups, a more preferred group of compounds is that wherein R³ is —CONR⁴R⁵ where R⁴ and R⁵ together with the nitrogen atom to which they are attached form unsaturated bicyclic heterocycloalkylamino, preferably, 1,2,3,4-tetrahydro-isoquinolin-2-yl.

Within the above preferred, more preferred, even more preferred groups, particularly and more particularly preferred groups, a more preferred group of compounds is that wherein R³ is CONR⁴R⁵ where R⁴ and R⁵ together with the nitrogen atom to which they are attached form saturated bridged heterocycloalkylamino, preferably, 3-aza-bicyclo[3.2.2]nonan-3-yl.

Within the above preferred, more preferred, even more preferred groups, particularly and more particularly preferred groups, a more preferred group of compounds is that wherein R³ is —CONR⁴R⁵ where R⁴ and R⁵ together with the nitrogen atom to which they are attached form unsaturated bridged heterocycloalkylamino, preferably, 3-aza-bicyclo[2.2.1]hept-5-en-3-yl.

2. Another preferred group of compounds of Formula I and II is that wherein:
R³ is —CONR⁴R⁵ where R⁴ and R⁵ together with the nitrogen atom to which they are attached form piperidin-1-yl optionally substituted with one, two, or three groups independently selected from hydrogen, alkyl, halo, hydroxy, alkoxy, alkoxycarbonyl, carboxy, haloalkyl, {[(CH₃)₃C]O(CO)NH}{[(CH₃)₃C]O(CO)CH₂}CH(CO)NH, alkylcarbonyloxy, —OSO₂OH, cycloalkylcarbonyloxy, or hydroxyalkyl; preferably, hydrogen, fluoro, bromo, chloro, hydroxy, methyl, methoxy, methoxycarbonyl, ethoxycarbonyl, carboxy, methylcarbonyloxy, {[(CH₃)₃C]O(CO)NH}{[(CH₃)₃C]O(CO)CH₂}CH(CO)NH, —OSO₂₀H, hydroxymethyl, hydroxyethyl, trifluoromethyl, or cyclohexylcarbonyloxy; more preferably, piperidin-1-yl, 3,3-difluoropiperidin-1-yl, 2-hydroxymethylpiperidin-1-yl, 3-hydroxymethylpiperidin-1-yl, 3-hydroxypiperidin-1-yl, 3R-hydroxypiperidin-1-yl, 4-hydroxypiperidin-1-yl, 3,3-difluoro-4-hydroxypiperidin-1-yl, 2-methylpiperidin-1-yl, 3-methylpiperidin-1-yl, 4-methylpiperidin-1-yl, 4-hydroxymethylpiperidin-1-yl, 4-bromopiperidin-1-yl, 2,6-dimethylpiperidin-1-yl, 3,3-dimethylpiperidin-1-yl, 3-methoxypiperidin-1-yl, 4-methoxypiperidin-1-yl, 3-fluoropiperidin-1-yl, 4-fluoropiperidin-1-yl, 4,4-difluoropiperidin-1-yl, 4-chloropiperidin-1-yl, 3-chloropiperidin-1-yl, 4-methoxycarbonylpiperidin-1-yl, 4-carboxypiperidin-1-yl, 4-ethoxycarbonylpiperidin-1-yl, 2-methoxycarbonylpiperidin-1-yl, 3,4-dihydroxypiperidin-1-yl, cis-3,4-dihydroxypiperidin-1-yl, trans-3,4-dihydroxypiperidin-1-yl, 3,5-dimethylpiperidin-1-yl, 2-hydroxyethylpiperidin-1-yl, 4-cyclohexylcarbonyloxypiperidin-1-yl,3,4-difluoropiperidin-1-yl, 3-trifluoromethylpiperidin-1-yl, 3-fluoro-4-hydroxypiperidin-1-yl, 4-methylcarbonyloxypiperidin-1-yl, 4-trifluoromethyl-piperidin-1-yl, 4-(HOSO₂O—)-3-hydroxypiperidin-1-yl, 4-acetyloxypiperidin-1-yl, cis-3-hydroxy-4-hydroxymethylpiperidin-1-yl, trans-3-hydroxy-4-hydroxymethylpiperidin-1-yl, cis-4-hydroxy-3-hydroxymethylpiperidin-1-yl, or 3-({[(CH₃)₃C]O(CO)NH}{[(CH₃)₃C]O(CO)CH₂}CH(CO)NH)-4-hydroxypiperidin-1-yl; even more preferably, piperidin-1-yl, 3,3-difluoropiperidin-1-yl, 4-hydroxypiperidin-1-yl, 3-hydroxypiperidin-1-yl, or 3,3-difluoro-4-hydroxypiperidin-1-yl.

3. Another preferred group of compounds of Formula I and II is that wherein:
R³ is —CONR⁴R⁵ where R⁴ and R⁵ together with the nitrogen atom to which they are attached form piperidin-1-yl substituted with one, two, or three groups independently selected from halo, alkyl, hydroxy, alkoxy, alkoxycarbonyl, carboxy, haloalkyl, alkylcarbonyloxy, cycloalkylcarbonyloxy, —OSO₂OH, {[(CH₃)₃C]O(CO)NH}{[(CH₃)₃C]O(CO)CH₂}CH(CO)NH, or hydroxyalkyl, provided that piperidin-1-yl is not substituted with halo or alkyl alone or any combination thereof; preferably, piperidin-1-yl substituted with fluoro, bromo, chloro, methyl, hydroxy, methoxy, methoxycarbonyl, ethoxycarbonyl, carboxy, methylcarbonyloxy, —OSO₂OH, hydroxymethyl, {[(CH₃)₃C]O(CO)NH}{[(CH₃)₃C]O(CO)CH₂}CH(CO)NH, hydroxyethyl, trifluoromethyl, or cyclohexylcarbonyloxy; more preferably, 2-hydroxymethylpiperidin-1-yl, 3-hydroxymethylpiperidin-1-yl, 3-hydroxypiperidin-1-yl, 3R-hydroxypiperidin-1-yl, 4-hydroxypiperidin-1-yl, 3,3-difluoro-4-hydroxypiperidin-1-yl, 4-hydroxymethylpiperidin-1-yl, 3-methoxypiperidin-1-yl, 4-methoxypiperidin-1-yl, 4-methoxycarbonylpiperidin-1-yl, 4-carboxypiperidin-1-yl, 4-ethoxycarbonylpiperidin-1-yl, 2-methoxycarbonylpiperidin-1-yl, 3,4-dihydroxypiperidin-1-yl, 2-hydroxyethylpiperidin-1-yl, 3-trifluoromethylpiperidin-1-yl, 4-trifluoromethyl-piperidin-1-yl, 4-methylcarbonyloxypiperidin-1-yl, 3-fluoro-4-hydroxypiperidin-1-yl, 4-cyclohexylcarbonyloxypiperidin-1-yl, 4-(HOSO₂O—)-3-hydroxypiperidin-1-yl, 4-acetyloxypiperidin-1-yl, cis-3,4-dihydroxypiperidin-1-yl, trans-3,4-dihydroxypiperidin-1-yl, cis-3-hydroxy-4-hydroxymethylpiperidin-1-yl, trans-3-hydroxy-4-hydroxymethylpiperidin-1-yl, cis-4-hydroxy-3-hydroxymethylpiperidin-1-yl, or 3-({[(CH₃)₃C]O(CO)NH}{[(CH₃)₃C]O(CO)CH₂}CH(CO)NH)-4-hydroxypiperidin-1-yl; even more preferably, 4-hydroxypiperidin-1-yl, 3-hydroxypiperidin-1-yl, or 3,3-difluoro-4-hydroxypiperidin-1-yl.

4. Another preferred group of compounds of Formula I or II is that wherein —X— is alkylene, Y is —NHCO—, Z is alkylene, R² is hydrogen, R¹ and R^{1a} are both hydrogen or halo, and Ar¹ is aryl. Preferably, X is methylene and Z is methylene, ethylene, or propylene which is optionally substituted with hydrogen, fluoro, hydroxy, difluoro, carboxy, amino, hydroxymethyl, ethoxycarbonyl, methylaminocarbonyl, or dimethylaminocarbonyl. More preferably, X and Y are methylene.

5. Another preferred group of compounds of Formula I or II is that wherein —X— is alkylene, Y is —NHCO—, Z is alkylene which is substituted with one or two hydrogen, halo, hydroxy, hydroxyalkyl, carboxy, amino, alkoxycarbonyl, alkylaminocarbonyl, or dialkylaminocarbonyl, R² is hydrogen, R¹ and R^{1a} are both hydrogen or halo, and Ar¹ is aryl. Preferably, X is methylene and Z is methylene, ethylene, or propylene which is optionally substituted with hydrogen, fluoro, hydroxy, difluoro, carboxy, amino, hydroxymethyl, ethoxycarbonyl, methylaminocarbonyl, or dimethylaminocarbonyl. More preferably, X and Y are methylene.

6. Another preferred group of compounds of Formula I or II is that wherein —X— is alkylene, Y is —NHCO—, Z is alkylene, R² is hydrogen, R¹ and R^{1a} are both hydrogen or halo, and Ar¹ is heteroaryl, preferably thien-3-yl. Preferably, X is methylene and Z is methylene, ethylene, or propylene which is optionally substituted with hydrogen, fluoro, hydroxy, difluoro, carboxy, amino, hydroxymethyl, ethoxycarbonyl, methylaminocarbonyl, or dimethylaminocarbonyl. More preferably, X is methylene and Y is methylene or difluoromethylene.

7. Another preferred group of compounds of Formula I or II is that wherein —X— is alkylene, Y is —NHCO—, Z is alkylene which is substituted with one or two hydrogen, halo, hydroxy, hydroxyalkyl, carboxy, amino, alkoxycarbonyl, alkylaminocarbonyl, or dialkylaminocarbonyl, R² is hydrogen, R¹ and R^{1a} are both hydrogen or halo, and Ar¹ is heteroaryl, preferably thien-3-yl. Preferably, X is methylene and Z is methylene, ethylene, or propylene which is optionally substituted with hydrogen, fluoro, hydroxy, difluoro, carboxy, amino, hydroxymethyl, ethoxycarbonyl, methylaminocarbonyl, or dimethylaminocarbonyl. More preferably, X is methylene and Y is methylene or difluoromethylene.

8. Another preferred group of compounds of Formula I or II is that wherein:

Het is thiazol-2-yl and is located at the 4-position of the phenylene ring; and Y is —NR⁷CO— or —NR⁷SO₂—, preferably —NHCO— or —NHSO₂—.

Within the above preferred group, a more preferred group of compounds is that wherein:
R² is hydrogen; and
R¹ and R^{1a} are hydrogen; and
R³ is —CONR⁴R⁵ where R⁴ and R⁵ together with the nitrogen atom to which they are attached form saturated heterocycloalkylamino, preferably, piperidin-1-yl; or

Within the above preferred, more preferred groups, and even more preferred groups of compounds is that wherein:
R³ is attached to the 4-position of the thiazol-2-yl; and
X is methylene; and
Z is alkylene, preferably, methylene or ethylene; and

Ar¹ is either:
i) phenyl; or
ii) heteroaryl, preferably, thien-2-yl.

9. Another preferred group of compounds of Formula I or II is that wherein: Het is 1H-pyrazol-1-yl and is located at the 4-position of the phenylene ring; and Y is —NR⁷CO—, preferably —NHCO—
R² is hydrogen; and
R¹ and R^{1a} are hydrogen; and
R³ is —CONR⁴R⁵ where R⁴ and R⁵ together with the nitrogen atom to which they are attached form saturated heterocycloalkylamino, preferably:
piperidin-1-yl, optionally substituted with hydrogen, hydroxy or halo, preferably hydrogen, hydroxy, or fluoro, more preferably piperidin-1-yl, 3,3-difluoropiperidin-1-yl, 3-hydroxypiperidin-1-yl, 4-hydroxypiperidin-1-yl; or

Within the above preferred, more preferred groups, and even more preferred groups of compounds is that wherein:
R³ is attached to the 4-position of the 1H-pyrazol-1-yl; and
X and Z are independently methylene; and
Ar¹ is either:
   i) phenyl; or
   ii) heteroaryl, preferably, thien-3-yl.

10. Another preferred group of compounds of Formula I or II is that wherein:
Het is 1H-imidazol-2-yl and is located at the 4-position of the phenylene ring; and Y is —NR⁷CO—, preferably —NHCO—.
R is hydrogen; and
R¹ and R^{1a} are hydrogen; and
R³ is —CONR⁴R⁵ where R⁴ and R⁵ together with the nitrogen atom to which they are attached form saturated heterocycloalkylamino, preferably:
piperidin-1-yl, optionally substituted with hydrogen or halo, preferably, hydrogen or fluoro, more preferably, piperidin-1-yl, 3,3-difluoropiperidin-1-yl; or
pyrrolidin-1-yl, optionally substituted with alkyl, preferably methyl, more preferably, 2,5-dimethylpyrrolidin-1-yl; or

Within the above preferred, more preferred groups, and even more preferred groups of compounds is that wherein:
R³ is attached to the 4-position of the 1H-imidazol-2-yl; and
X and Z are independently methylene; and
Ar¹ is phenyl.

11. Another preferred group of compounds of Formula I or II is that wherein:
Het is [1,2,4]oxadiazol-3-yl and is located at the 4-position of the phenylene ring; and
Y is —NR⁷CO—, preferably-NHCO—,
R¹ and R^{1a} are hydrogen; and
R³ is —CONR⁴R⁵ where R⁴ and R⁵ together with the nitrogen atom to which they are attached form saturated heterocycloalkylamino, preferably: piperidin-1-yl; or

Within the above preferred, more preferred, and even more preferred groups of compounds is that wherein:
R³ is attached to the 5-position of the [1,2,4]oxadiazol-3-yl; and
X and Z are independently methylene; and
Ar¹ is phenyl.

12. Another preferred group of compounds of Formula I or II is that wherein Y is —O—, —NR⁶—, —S—, —SO—, —SO₂—, —NR⁷CO—, —NR⁷SO₂—, —SO₂NR⁷—, —NHCONH—, —NHCSNH—, or —NHCOO—.

13. Another preferred group of compounds of Formula I or II is that wherein:

Ar¹ is aryl substituted with one, two, or three subsitutents independently selected from alkyl, halo, alkoxy, methylenedioxy, azido, haloalkyl, hydroxy, or haloalkoxy; preferably aryl substituted with one, two, or three substituents independently selected from methyl, chloro, fluoro, iodo, methoxy, methylenedioxy, trifluoromethyl, azido, hydroxy, or trifluoromethoxy.

Within the above preferred, more preferred, and even more preferred groups of compounds is that wherein:

Ar¹ is phenyl substituted with one, two, or three subsitutents independently selected from alkyl, halo, alkoxy, methylenedioxy, azido, haloalkyl, hydroxy, or haloalkoxy; preferably phenyl optionally substituted with one, two, or three substituents independently selected from methyl, chloro, fluoro, iodo, methoxy, methylenedioxy, trifluoromethyl, azido, hydroxy, or trifluoromethoxy. More preferably, Ar¹ is phenyl, 2-methylphenyl, 3-methylphenyl, 4-methylphenyl, 2-methoxyphenyl, 3-methoxyphenyl, 4-methoxyphenyl, 2-fluorophenyl, 3-fluorophenyl, 4-fluorophenyl, 3-chlorophenyl, 4-chlorophenyl, 2,6-dichlorophenyl, 3,4-dichlorophenyl, 2,4-dichlorophenyl, 4-trifluoromethoxyphenyl, 3,4-dimethoxyphenyl, 2,5-dimethoxyphenyl, 4-ethoxy-phenyl, 3,5-dimethylphenyl, 3,4-difluorophenyl, 2,5-bis-(trifluoromethyl)phenyl, 3,4-methylenedioxyphenyl, 4-methoxy-3-methylphenyl, 3,4,5-trimethoxyphenyl, 3-azidophenyl, 4-azidophenyl, 4-iodophenyl, 2-hydroxyphenyl, 3-hydroxyphenyl, 4-hydroxyphenyl, or 3-fluoro-4-hydroxyphenyl. Even more preferably Ar¹ is phenyl, 4-trifluoromethoxyphenyl, 4-chlorophenyl, or 2-fluorophenyl. 14. Another preferred group of compounds of Formula I or II is that wherein Ar¹ is heteroaryl substituted with one, two, or three substituents independently selected from the group consisting of alkyl, alkoxy, alkylthio, haloalkyl, haloalkoxy, halo, hydroxy, amino, alkylamino, dialkylamino, nitro, alkylcarbonyl, alkylcarbonylamino, alkoxycarbonyl, alkoxyalkyl, aminoalkyl, aminocarbonyl, alkylaminocarbonyl, dialkylaminocarbonyl, carboxy, cyano, hydroxyalkyl, or optionally substituted phenyl.

15. Another preferred group of compounds of Formula I or II is that wherein Ar¹ is unsubstituted heteroaryl, preferably pyridinyl, thienyl, 3-methyl-isoxazol-5-yl, or furanyl; more preferably thien-2-yl, thien-3-yl, pyridin-2-yl, pyridin-3-yl, 3-methyl-isoxazol-5-yl, or furan-2-yl; even more preferably thien-3-yl.

Reference to the preferred embodiments set forth above is meant to include all combinations of particular and preferred groups unless stated otherwise. A person of ordinary skill in the art would recognize that certain groups listed above in the preferred embodiments can exist as geometric or stereoisomers. The present invention includes individual stereoisomers and geometric isomers and mixtures thereof.

Representative compounds of Formulae I and II where R¹ and R^{1a} are hydrogen and other groups are as specified below are:

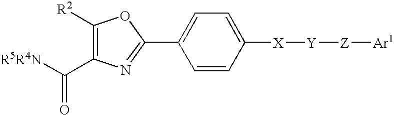

| Cmpd. | | | | | | |
|---|---|---|---|---|---|---|
| No. | —NR⁴R⁵ | R | X | Y | Z | Ar¹ |
| 22198 | 3,3-difluoropieridin-1- | H | CH₂ | NHCO | CH₂ | phenyl |
| | yl | | | | | |
| 19242 | piperidin-1-yl | H | CH₂ | NHCO | CH₂ | 4-(trifluoromethoxy)- |
| | | | | | | phenyl |
| 22221 | 4-hydroxypiperidin-1- | H | CH₂ | NHCO | CHF | phenyl |
| | yl | | | | | |
| 20991 | 3-hydroxypiperidin-1- | H | CH₂ | NHCO | CH₂ | phenyl |
| | yl | | | | | |
| 21817 | piperidin-1-yl | H | CH₂ | NHCO | CH₂ | 4-chlorophenyl |
| 21821 | 3-hydroxypiperidin-1- | H | CH₂ | NHCO | CH₂ | thien-3-yl |
| | yl | | | | | |
| 22538 | 3,3-difluoropiperidin-1- | H | CH₂ | NHCO | CH₂ | thien-3-yl |
| | yl | | | | | |
| 21932 | homopiperidin-1-yl | H | CH₂ | NHCO | CH₂ | thien-3-yl |
| 19241 | piperidin-1-yl | H | CH₂ | NHCO | CH₂ | phenyl |
| 21552 | piperidin-1-yl | H | CH₂ | NHCO | CHF | phenyl |
| 19237 | piperidin-1-yl | H | CH₂ | NHCO | CH₂ | thien-3-yl |
| 23186 | piperidin-1-yl | H | CH₂ | NHCO | CF₂ | thien-3-yl |
| 21003 | homopiperidin-1-yl | H | CH₂ | NHCO | CH₂ | phenyl |
| 23953 | 4- | H | CH₂ | NHCO | CF₂ | thien-2-yl |
| | hydroxyhomopiperidin- | | | | | |
| | 1-yl | | | | | |
| 23731 | 3-hydroxypiperidin-1- | H | CH₂ | NHCO | CF₂ | thien-2-yl |
| | yl | | | | | |
| 23182 | 4- | H | CH₂ | NHCO | CH₂ | thien-3-yl |
| | hydroxyhomopiperidin- | | | | | |
| | 1-yl | | | | | |
| 23383 | 3,3-difluoro-4- | H | CH₂ | NHCO | CH₂ | thien-3-yl |
| | hydroxypiperidin-1-yl | | | | | |
| 23277 | piperidin-1-yl | H | CH₂ | NHCO | CHF | 2-fluorophenyl |
| 19010 | piperidin-1-yl | H | CH₂ | NHCO | CH₂CH₂ | 2-methoxyphenyl |
| 19011 | piperidin-1-yl | H | CH₂ | NHCO | CH₂CH₂ | 3-methoxyphenyl |
| 19012 | piperidin-1-yl | H | CH₂ | NHCO | CH₂CH₂ | 4-methoxyphenyl |
| 19015 | piperidin-1-yl | H | CH₂ | NHCO | CH₂CH₂ | 4-methylphenyl |
| 19016 | piperidin-1-yl | H | CH₂ | NHCO | CH₂CH₂ | 3,4-difluorophenyl |
| 19021 | piperidin-1-yl | H | CH₂ | NHCO | CH₂CH₂ | 2,5-his- |
| | | | | | | (trifluoromethyl)- |
| | | | | | | phenyl |
| 19024 | piperidin-1-yl | H | CH₂ | NHCO | CH₂CH₂ | 3-fluorophenyl |
| 19025 | piperidin-1-yl | H | CH₂ | NHCO | CH₂CH₂ | 2-fluorophenyl |
| 19026 | piperidin- l-yl | H | CH₂ | NHCO | CH₂CH₂ | 3,4- |
| | | | | | | methylenedioxyphenyl |
| 19027 | piperidin-1-yl | H | CH₂ | NHCO | CH₂CH₂ | 3,4-dichlorophenyl |
| 19028 | piperidin-1-yl | H | CH₂ | NHCO | CH₂CH₂ | 2,6-dichlorophenyl |
| 19029 | piperidin-1-yl | H | CH₂ | NHCO | CH₂CH₂ | 3-methylphenyl |
| 19030 | piperidin-1-yl | H | CH₂ | NHCO | CH₂CH₂ | 4-fluorophenyl |
| 19031 | piperidin-1-yl | H | CH₂ | NHCO | CH₂CH₂ | 2,4-dichlorophenyl |
| 19032 | piperidin-1-yl | H | CH₂ | NHCO | CH₂CH₂ | 2,5-dimethoxyphenyl |
| 19033 | piperidin-1-yl | H | CH₂ | NHCO | (CH₃)CHCH₂ | phenyl |
| 19034 | piperidin-1-yl | H | CH₂ | NHCO | CH₂CH(CH₃) | phenyl |
| 19035 | piperidin-1-yl | H | CH₂ | NHCO | CH₂CH₂ | 2-methylphenyl |
| 19243 | piperidin-1-yl | H | CH₂ | NHCO | CH₂ | 3,4- |
| | | | | | | methylenedioxyphenyl |
| 19329 | piperidin-1-yl | H | CH₂ | NHCO | CH₂ | 4-methoxy-3- |
| | | | | | | methylphenyl |
| 19330 | piperidin-1-yl | H | CH₂ | NHCO | CH₂ | 3,4,5- |
| | | | | | | trimethoxyphenyl |
| 19336 | piperidin-1-yl | H | CH₂ | NHCO | CH₂ | 4-methylphenyl |
| 19345 | piperidin-1-yl | H | CH₂ | NHCO | CH₂ | pyridin-2-yl |
| 19354 | piperidin-1-yl | H | CH₂ | NHCO | CH₂ | 3,4-dimethoxyphenyl |
| 19380 | piperidin-1-yl | H | CH₂ | NHCO | CH₂CH₂ | pyridin-2-yl |
| 19509 | 2-methylpiperidin-1-yl | H | CH₂ | NHCO | CH₂ | 4-methoxyphenyl |
| 19510 | 2-methylpiperidin-1-yl | H | CH₂ | NHCO | CH₂CH₂ | phenyl |
| 19512 | piperidin-1-yl | H | CH₂ | NHCO | CH₂ | 4-ethoxyphenyl |
| 19518 | 3-methylpiperidin-1-yl | H | CH₂ | NHCO | CH₂ | 4-methoxyphenyl |
| 19520 | piperidin-1-yl | H | CH₂ | NHCO | CH₂CH₂ | furan-2-yl |
| 19521 | 3-methylpiperidin-1-yl | H | CH₂ | NHCO | CH₂CH₂ | phenyl |
| 19526 | piperidin-1-yl | H | CH₂ | NHCO | CH₂CH₂CH₂ | thien-2-yl |
| 19534 | piperidin-1-yl | H | CH₂ | NICO | CH₂ | pyridin-3-yl |
| 19544 | piperidin-1-yl | H | CH₂ | NHCO | CH₂ | 3,5-dimethylphenyl |
| 19939 | piperidin-1-yl | H | CH₂ | NHCO | CH₂CH₂ | thien-2-yl |
| 20140 | 3-methylpiperidin-1-yl | H | CH₂ | NHCO | CH₂ | thien-2-yl |
| 20142 | thiomorpholin-4-yl | H | CH₂ | NHCO | CH₂ | thien-2-yl |
| 20144 | 1,4-dioxa-8-aza- | H | CH₂ | NHCO | CH₂ | thien-2-yl |
| | spiror[4.5]decan-8-yl | | | | | |
| 20147 | 2,6-dimethylpiperidin- | H | CH₂ | NHCO | CH₂ | thien-2-yl |
| | 1-yl | | | | | |
| 20150 | 2-methylpiperidin-1-yl | H | CH₂ | NHCO | CH₂ | thien-2-yl |
| 20153 | 3,5-dimethylpiperidin- | H | CH₂ | NHCO | CH₂CH₂ | phenyl |
| | 1-yl | | | | | |
| 20154 | 4- | H | CH₂ | NHCO | CH₂CH₂ | phenyl |
| | hydroxymethylpiperidin- | | | | | |
| | 1-yl | | | | | |
| 20158 | 4-hydroxypiperidin-1- | H | CH₂ | NHCO | CH₂ | 4-methoxyphenyl |
| | yl | | | | | |
| 20315 | piperidin-1-yl | H | CH₂ | N(CH₃)CO | (CH₃)₂C | phenyl |
| 20317 | piperidin-1-yl | H | CH₂ | N(CH₃)CO | CH₂ | phenyl |
| 20427 | thiomorpholin-4-yl | H | CH₂ | NHCO | CH₂ | 4-methoxyphenyl |
| 20641 | 1,2,3,6-tetrahydro- | H | CH₂ | NHCO | CH₂ | 4-methoxyphenyl |
| | pyridin-1-yl | | | | | |
| 20642 | piperidin-1-yl | H | CH₂ | NHCO | CF₂ | phenyl |
| 20643 | pyrrolidin-1-yl | H | CH₂ | NHCO | CH₂ | thien-2-yl |
| 20645 | 4-bromopiperidin-1-yl | H | CH₂ | NHCO | CH₂ | thien-2-yl |
| 20646 | 1,2,3,6-tetrahydro- | H | CH₂ | NHCO | CH₂ | thien-2-yl |
| | pyridin-1-yl | | | | | |
| 20647 | homopiperidin-1-yl | H | CH₂ | NHCO | CH₂ | 4-methoxyphenyl |
| 20648 | 4- | H | CH₂ | NHCO | CH₂ | 4-methoxyphenyl |
| | hydroxymethylpiperidin- | | | | | |
| | 1-yl | | | | | |
| 20661 | 2,6-dimethylpiperidin- | H | CH₂ | NHCO | CH₂CH₂ | phenyl |
| | 1-yl | | | | | |
| 20878 | piperidin-1-yl | CH₃ | CH₂ | NHCO | CH₂ | phenyl |
| 20879 | piperidin-1-yl | CH₃ | CH₂ | NHCO | CH₂ | thien-2-yl |
| 21543 | 2,6-dimethylpiperidin- | H | CH₂ | NHCO | CH₂ | 4-methoxyphenyl |
| | 1-yl | | | | | |
| 20986 | 4-methylpiperidin-1-yl | H | CH₂ | NHCO | CH₂ | phenyl |
| 20987 | 2-methylpiperidin-1-yl | H | CH₂ | NHCO | CH₂ | phenyl |
| 20988 | 4-hydroxypiperidin-1- | H | CH₂ | NHCO | CH₂ | phenyl |
| | yl | | | | | |
| 20989 | thiomorpholin-4-yl | H | CH₂ | NHCO | CH₂ | phenyl |
| 20990 | 3-methylpiperidin-1-yl | H | CH₂ | NHCO | CH₂ | phenyl |
| 20992 | morpholin-4-yl | H | CH₂ | NHCO | CH₂ | phenyl |
| 20993 | 4- | H | CH₂ | NHCO | CH₂ | phenyl |
| | hydroxymethyl- | | | | | |
| | piperidin-1-yl | | | | | |
| 20995 | 4-bromopiperidin-1-yl | H | CH₂ | NHCO | CH₂ | phenyl |
| 20997 | 1,2,3,6-tetrahydro- | H | CH₂ | NHCO | CH₂ | phenyl |
| | pyridin-1-yl | | | | | |
| 20998 | pyrrolidin-1-yl | H | CH₂ | NHCO | CH₂ | phenyl |
| 21000 | 2-methylpiperidin-1-yl | CH₃ | CH₂ | NHCO | CH₂ | phenyl |
| 21001 | 2-methylpiperidin-1-yl | CH₃ | CH₂ | NHCO | CH₂ | thien-2-yl |
| 21543 | 2,6-dimethylpiperidin- | H | CH₂ | NHCO | CH₂ | 4-methoxyphenyl |
| | 1-yl | | | | | |
| 21547 | homopiperidin-1-yl | H | CH₂ | NHCO | CF₂ | phenyl |
| 21548 | 1,2,3,6-tetrahydro- | H | CH₂ | NHCO | CF₂ | phenyl |
| | pyridin-1-yl | | | | | |
| 21553 | homopiperidin-1-yl | H | CH₂ | NHCO | CHF | phenyl |
| 21554 | 1,2,3,6-tetrahydro- | H | CH₂ | NHCO | CHF | phenyl |
| | pyridin-1-yl | | | | | |
| 21555 | pyrrolidin-1-yl | H | CH₂ | NHCO | CHF | phenyl |
| 21811 | piperidin-1-yl | H | CH₂ | NHCO | CH₂ | 2-fluorophenyl |
| 21812 | piperidin-1-yl | H | CH₂ | NHCO | CH₂ | 3-fluorophenyl |
| 21813 | piperidin-1-yl | H | CH₂ | NHCO | CH₂ | 4-fluorophenyl |
| 21815 | piperidin-1-yl | H | CH₂ | NHCO | CH₂ | 2,6-difluorophenyl |
| 21816 | piperidin-1-yl | H | CH₂ | NHCO | CH₂ | 3-chiorophenyl |
| 21830 | piperidin-1-yl | H | CH₂CH₂ | NHCO | CH₂ | phenyl |
| 21931 | piperidin-1-yl | H | CH₂ | NHCO | CH₂ | furan-2-yl |
| 22041 | 3-hydroxypiperidin-1- | H | CH₂ | NHCO | CH₂ | 4- |
| | yl | | | | | trifluoromethoxy- |
| | | | | | | phenyl |
| 22042 | 4-hydroxy piperidin-1- | H | CH₂ | NHCO | CH₂ | 4- |
| | yl | | | | | trifluoromethoxyphen |
| | | | | | | yl |
| 22108 | piperidin-1-yl | Br | CH₂ | NHCO | CH₂ | phenyl |
| 22109 | azetidin-1-yl | H | CH₂ | NHCO | CH₂ | phenyl |
| 22110 | 2-methylpyrrolidin-1-yl | H | CH₂ | NHCO | CH₂ | phenyl |
| 22111 | 3-hydroxy-pyrrolidin-1- | H | CH₂ | NHCO | CH₂ | phenyl |
| | yl | | | | | |
| 22112 | 2,5-dimethyl- | H | CH₂ | NHCO | CH₂ | phenyl |
| | pyrrolidin-1-yl | | | | | |
| 22113 | trans-2,5-dimethyl-2,5- | H | CH₂ | NHCO | CH₂ | phenyl |
| | dihydro-1H-pyrrol-1-yl | | | | | |
| 22114 | thiazolidin-3-yl | H | CH₂ | NHCO | CH₂ | phenyl |
| 22115 | 2-methylthiazolidin-3- | H | CH₂ | NHCO | CH₂ | phenyl |
| | yl | | | | | |
| 22116 | 3-dimethyl-piperidin- | H | CH₂ | NHCO | CH₂ | phenyl |
| | 1-yl- | | | | | |
| 22117 | piperazin-1-yl- | H | CH₂ | NHCO | CH₂ | phenyl |
| 22120 | 4-acetylpiperazin-1-yl | H | CH₂ | NHCO | CH₂ | phenyl |
| 22121 | 1-oxothio-moriholin-4- | H | CH₂ | NHCO | CH₂ | phenyl |
| | yl | | | | | |
| 22122 | 1,1-dioxothio- | H | CH₂ | NHCO | CH₂ | phenyl |
| | morpholin-4-yl | | | | | |
| 22123 | 3-hydroxypiperidin-1- | H | CH₂ | (NCH₃)CO | CH₂ | phenyl |
| | yl | | | | | |
| 22124 | 3-methoxy-piperidin-1- | H | CH₂ | NHCO | CH₂ | phenyl |
| 22125 | 3-methoxy-piperidin-1- | H | CH₂ | (NCH₃)CO | CH₂ | phenyl |
| | yl | | | | | |
| 22126 | 4-hydroxypiperidin-1- | H | CH₂ | (NCH₃)CO | CH₂ | phenyl |
| | yl | | | | | |
| 22127 | 4-methoxy-piperidin-1- | H | CH₂ | NHCO | CH₂ | phenyl |
| | yl- | | | | | |
| 22128 | 4-methoxy-piperidin-1- | H | CH | (NCH₃)CO | CH₂ | phenyl |
| | yl- | | | | | |
| 22129 | homopiperazin-1-yl | H | CH₂ | NHCO | CH₂ | phenyl |
| 22130 | 4-methylhomo- | H | CH₂ | NHCO | CH₂ | phenyl |
| | piperazin-1-yl | | | | | |
| 22131 | azocan-1-yl- | H | CH₂ | NHCO | CH₂ | phenyl |
| 22132 | 1,2,3,4-tetrahydro- | H | CH₂ | NHCO | CH₂ | phenyl |
| | isoquinolin-2-yl- | | | | | |
| 22134 | decahydro-isoquinolin- | H | CH₂ | NICO | CH₂ | phenyl |
| | 2-yl- | | | | | |
| 22136 | 3-aza-bicyclo[2.2.1]- | H | CH₂ | NHCO | CH₂ | phenyl |
| | hept-5-en-3-yl | | | | | |
| 22137 | 3-aza-bicyclo[3.2.2]- | H | CH₂ | NICO | CH₂ | phenyl |
| | non-6-ene-3-yl | | | | | |
| 22194 | 4-fluoropiperidin-1-yl | H | CH₂ | NHCO | CH₂ | phenyl |
| 22195 | 4,4-difluoropiperidin-1- | H | CH₂ | NHCO | CH₂ | phenyl |
| | yl- | | | | | |
| 22196 | 2-methylaziridin-1-yl | H | CH₂ | NHCO | CH₂ | phenyl |
| 22212 | 4-hydroxymethyl- | H | CH₂ | NHCO | CH₂ | 4- |
| | piperidin-1-yl- | | | | | trifluoromethoxyphenyl |
| 22222 | 3-hydroxypiperidin-1- | H | CH₂ | NHCO | CHF | phenyl |
| | yl | | | | | |
| 22223 | 4-hydroxymethyl- | H | CH₂ | NHCO | CHF | phenyl |
| | piperidin-1-yl | | | | | |
| 22224 | 4-hydroxypiperidin-1- | H | CH₂ | NHCO | CH₂ | thien-3-yl |
| | yl | | | | | |
| 22226 | 4-hydroxypipendin-1- | H | CH₂ | NHCO | CH₂ | furan-2-yl |
| | yl | | | | | |
| 22227 | 3-hydroxypiperidin-1- | H | CH₂ | NHCO | CH₂ | furan-2-yl |
| | yl | | | | | |
| 22228 | 4-hydroxymethyl- | H | CH₂ | NHCO | CH₂ | furan-2-yl |
| | piperidin-1-yl | | | | | |
| 22230 | 3-fluoropiperidin-1-yl | H | CH₂ | NHCO | CH₂ | phenyl |
| 22231 | 4-oxopiperidin-1-yl | H | CH₂ | NHCO | CH₂ | phenyl |
| 22258 | trans-2,5-dimethyl- | H | CH₂ | NHCO | CH₂ | phenyl |
| | piperazin-1-yl | | | | | |
| 22324 | 3-oxopiperidin-1-yl | H | CH₂ | NUCO | CH₂ | phenyl |
| 22325 | 4-chloropiperidin-1-yl | H | CH₂ | NHCO | CH₂ | phenyl |
| 22327 | 3-chloropiperidin-1-yl | H | CH₂ | NHCO | CH₂ | phenyl |
| 22332 | 4-fluoropiperidin-1-yl | H | CH₂ | NHCO | CHF | phenyl |
| 22333 | 4-methoxycarbonyl- | H | CH₂ | NHCO | CHF | phenyl |
| | piperidrn-1-yl- | | | | | |
| 22334 | 4-carboxypiperidin-1-yl | H | CH₂ | NHCO | CHF | phenyl |
| 22335 | piperidin-1-yl- | H | CH₂ | NHSO₂ | CH₂ | phenyl |
| 22339 | 4-ethoxycarbonyl- | H | CH₂ | NHCO | CHF | phenyl |
| | piperidin-1-yl- | | | | | |
| 22350 | 2-methoxycarbonyl- | H | CH₂ | NHCO | CH₂ | phenyl |
| | piperidin-1-yl | | | | | |
| 22363 | 3,3-difluoropiperidin-1- | H | CH₂ | NHCO | CHF | phenyl |
| | yl | | | | | |
| 22390 | azocan-1-yl | H | CH₂ | NHCO | CHF | phenyl |
| 22392 | 2-methylpyrrolidin-1-yl | H | CH₂ | NHCO | CHF | phenyl |
| 22396 | morpholin-4-y-1 | H | CH₂ | NHCO | CH₂ | 4- |
| | | | | | | trifluoromethoxyphenyl |
| 22397 | morpholin-4-yl- | H | CH₂ | NHCO | CHF | phenyl |
| 22398 | morpholin-4-yl- | H | CH₂ | NHCO | CH₂ | thien-3-yl |
| 22429 | 2,5-dimethyl- | H | CH₂ | NHCO | CHF | phenyl |
| | pyrrolidin-1-yl- | | | | | |
| 22432 | 2S-methoxy-carbonyl- | H | CH₂ | NHCO | CH₂ | phenyl |
| | pyrrolidin-1-yl | | | | | |
| 22472 | 2S- | H | CH₂ | NHCO | CH₂ | phenyl |
| | hydroxymethylpyrrolidin- | | | | | |
| | 1-yl | | | | | |
| 22473 | 2R-hydroxy- | H | CH₂ | NHCO | CH₂ | phenyl |
| | methylpyrrolidin-1-yl | | | | | |
| 22490 | trans-2,5-dimethyl- | H | CH₂ | NHCO | CH₂ | phenyl |
| | pyrrolidin-1-y-l | | | | | |
| 22539 | 2,5-dimethyl- | H | CH₂ | NHCO | CH₂ | thien-3-yl |
| | pyrrolidin-1-yl- | | | | | |
| 22540 | 1,2,3,6-tetrahydro- | H | CH₂ | NHCO | CH₂ | thien-3-yl |
| | pyridin-1-yl | | | | | |
| 22541 | 3-methylpiperidin-1-yl | H | CH₂ | NHCO | CH₂ | thien-3-yl |
| 22542 | 2-methylpyrrolidin-1-yl | H | CH₂ | NHCO | CH₂ | thien-3-yl |
| 22568 | cis-3,4-dihydroxy- | H | CH₂ | NHCO | CH₂ | phenyl |
| | pipendin-1-yl | | | | | |
| 22597 | 3-chloropiperidin-1-yl | H | CH₂ | NHCO | CH₂ | thien-3-yl |
| 22598 | 4-chloropiperidin-1-yl | H | CH₂ | NHCO | CH₂ | thien-3-yl |
| 22599 | 3,5-dimethyl-piperidin- | H | CH₂ | NHCO | CH₂ | thien-3-yl |
| | 1-yl- | | | | | |
| 22600 | 2-(2-hydroxyethyl)- | H | CH₂ | NHCO | CH₂ | thien-3-yl |
| | piperidin-1-yl- | | | | | |
| 22602 | 2,6-dimethyl-piperidin- | H | CH₂ | NHCO | CH₂ | thien-3-yl |
| | 1-yl- | | | | | |
| 22605 | 4,4-difluoropiperdin-1- | H | CH₂ | NHCO | CH₂ | thien-3-yl |
| | yl- | | | | | |
| 22619 | 4-fluoropiperidin-1-yl | H | CH₂ | NHCO | CH₂ | thien-3-yl |
| 22877 | 3,4-difluoropiperidin-1- | H | CH₂ | NHCO | CH₂ | phenyl |
| | yl- | | | | | |
| 22878 | 3 -hydroxy-4- | H | CH₂ | NHCO | CH₂ | phenyl |
| | (OSO₂OH)-piperidin-1- | | | | | |
| | yl | | | | | |
| 22952 | 3-methoxy-piperidin-1- | H | CH₂ | NHCO | CH₂ | thien-3-yl |
| | yl- | | | | | |
| 22607 | 3-hydroxymethyl- | H | CH₂ | NHCO | CH₂ | thien-3-yl |
| | pipendin-1-yl | | | | | |
| 22608 | 2-hydroxymethy- | H | CH₂ | NHCO | CH₂ | thien-3-yl |
| | ipiperidin-1-yl | | | | | |
| 22972 | thiomorpholin-4-yl- | H | CH₂ | NHCO | CH₂ | thien-3-yl |
| 22974 | azocan-1-yl | H | CH₂ | NHCO | CH₂ | thien-3-yl |
| 22976 | 4-methylpiperidin-1-yl | H | CH₂ | NHCO | CH₂ | thien-3-yl |
| 22977 | 3-fluoropiperidin-1-yl | H | CH₂ | NHCO | CH₂ | thien-3-yl |
| 23178 | 3-hydroxy- | H | CH₂ | NHCO | CH₂ | phenyl |
| | (homopiperidin-1-yl) | | | | | |
| 23179 | 4-hydroxy- | H | CH₂ | NHCO | CH₂ | phenyl |
| | (homopiperidin-1-yl) | | | | | |
| 23180 | 3R-hydroxy-piperidin- | H | CH₂ | NHCO | CH₂ | phenyl |
| | 1-yl- | | | | | |
| 23181 | 3-hydroxy- | H | CH₂ | NHCO | CH₂ | thien-3-yl |
| | (homopiperidin-1-yl) | | | | | |
| 23183 | 3R-hydroxy-piperidin- | H | CH₂ | NHCO | CH₂ | thien-3-yl |
| | 1-yl- | | | | | |
| 23185 | 4-hydroxypiperidin-1- | H | CH₂ | NHCO | CF₂ | thien-3-yl |
| | yl | | | | | |
| 23187 | 3-trifluoromethyl- | H | CH₂ | NHCO | CH₂ | thien-3-yl |
| | piperidin-1-yl- | | | | | |
| 23188 | 3- | H | CH₂ | NHCO | CH₂ | phenyl |
| | trifluoromethylpiperidin- | | | | | |
| | 1-yl | | | | | |
| 23234 | 3,3-difluoropiperidin-1- | H | CH₂ | NHCO | CF₂ | thien-3-yl |
| | yl | | | | | |
| 23235 | 3,3-difluoropiperidin-1- | H | CH₂ | NHCO | CF₂ | thien-2-yl |
| | yl | | | | | |
| 23236 | piperidin-1-yl | H | CH₂ | NHCO | CF₂ | thien-2-yl |
| 23237 | 4-hydroxypiperidin-1- | H | CH₂ | NHCO | CF₂ | thien-2-yl |
| | yl | | | | | |
| 23239 | 4-hydroxypiperidin-1- | H | CH₂ | NHCO | CF₂ | phenyl |
| | yl | | | | | |
| 23240 | 3-hydroxypiperidin-1- | H | CH₂ | NHCO | CF₂ | phenyl |
| | yl | | | | | |
| 23241 | 3,3-difluoropiperidin-1- | H | CH₂ | NHCO | CF₂ | phenyl |
| | yl | | | | | |
| 23242 | trans-4-fluoro-3- | H | CH₂ | NHCO | CH₂ | phenyl |
| | hydroxy- | | | | | |
| | (homopiperidin-1-yl) | | | | | |
| 23243 | trans-4-fluoro-3- | H | CH₂ | NHCO | CH₂ | thien-3-yl |
| | hydroxy- | | | | | |
| | (homopiperidin-1-yl) | | | | | |
| 23249 | 3-fluoro-4- | H | CH₂ | NHCO | CH₂ | thien-3-yl |
| | hydroxypiperidin-1-yl | | | | | |
| 23261 | piperidin-1-yl | H | CH₂ | NHCO | CF₂ | pyridin-2-yl |
| 23262 | 4-hydroxypiperidin-1- | H | CH₂ | NHCO | CF₂ | pyridin-2-yl |
| | yl | | | | | |
| 23263 | piperidin-1-yl | H | CH₂ | NHCO | CHOH | phenyl |
| 23264 | 3,3-difluoropiperidin-1- | H | CH₂ | NHCO | CHOH | phenyl |
| | yl | | | | | |
| 23265 | 3,3-difluoropiperidin-1- | H | CH₂ | NHCO | R—CHOH | phenyl |
| | yl | | | | | |
| 23266 | 3-chloropiperidin-1-yl | H | CH₂ | NHCO | CHF | phenyl |
| 23267 | 3,3-difluoropiperidin-1- | H | CH₂ | NHCO | CH₂ | 2-fluorophenyl |
| | yl | | | | | |
| 23268 | 3,3-difluoropiperidin-1- | H | CH₂ | NHCO | CH₂ | 3-fluorophenyl |
| | yl | | | | | |
| 23269 | 4-hydroxypiperidin-1- | H | CH₂ | NHCO | CH₂ | 2-fluorophenyl |
| | yl | | | | | |
| 23270 | 4-hydroxypiperidin-1- | H | CH₂ | NHCO | CH₂ | 3-fluorophenyl |
| | yl | | | | | |
| 23271 | 3-hydroxypiperidin-1- | H | CH₂ | NHCO | CH₂ | 2-fluorophenyl |
| | yl | | | | | |
| 23272 | 3-hydroxypiperidin-1- | H | CH₂ | NHCO | CH₂ | 3-fluorophenyl |
| | yl | | | | | |
| 23273 | 3-fluoropiperidin-1-yl | H | CH₂ | NHCO | CHF | phenyl |
| 23274 | 3,3-difluoropiperidin-1- | H | CH₂ | NHCO | S—CHOH | phenyl |
| | yl | | | | | |
| 23275 | [1,3]oxazinan-3-yl | H | CH₂ | NHCO | CH₂ | thien-3-yl |
| 23276 | [1,3]oxazinan-3-yl | H | CH₂ | NHCO | CH₂ | phenyl |
| 23278 | 3-hydroxypiperidin-1- | H | CH₂ | NHCO | CHF | 2-fluorophenyl |
| | yl | | | | | |
| 23279 | 4-hydroxypiperidin-1- | H | CH₂ | NHCO | CHF | 2-fluorophenyl |
| | yl | | | | | |
| 23280 | 3,3-difluoropiperidin-1- | H | CH₂ | NHCO | CHF | 2-fluorophenyl |
| | yl | | | | | |
| 23460 | 3- | H | CH₂ | NHCO | CH₂ | phenyl |
| | {[(CH₃)₃C]O(CO)NH]} | | | | | |
| | {[(CH₃)₃C]O(CO)CH₂} | | | | | |
| | CH(CO)NH-4- | | | | | |
| | hydroxypiperidin-1-yl | | | | | |
| 23480 | piperidin-1-yl | H | CH₂ | NHCO | CH₂ | 4-azidophenyl |
| 23481 | piperidin-1-yl | H | CH₂ | NHCO | CH₂ | 3-azidophenyl |
| 23497 | trans-2,5-dimethyl- | H | CH₂ | NHCO | CH₂ | 4-iodophenyl |
| | pyrrolidin-1-yl | | | | | |
| 23498 | cis-2,5-dimethyl- | H | CH₂ | NHCO | CH₂ | 4-iodophenyl |
| | pyrrolidin-1-yl | | | | | |
| 23499 | trans-2,5-dimethyl-2,5- | H | CH₂ | NHCO | CH₂ | 4-iodophenyl |
| | dihydro-1H-pyrrol-1-yl | | | | | |
| 23500 | cis-2,5-dimethyl-2,5- | H | CH₂ | NHCO | CH₂ | 4-iodophenyl |
| | dihydro-1H-pyrrol-1-yl | | | | | |
| 23501 | cis-2,5-dimethyl-2,5- | H | CH₂ | NHCO | CH₂ | phenyl |
| | dihydro-1H-pyrrol-1-yl | | | | | |
| 23516 | 3-fluoro-4- | H | CH₂ | NHCO | CH₂ | thien-3-yl |
| | oxopiperidin-1-yl | | | | | |
| 23536 | piperidin-1-yl | H | CH₂ | NHCO | CH₂ | 2-hydroxyphenyl |
| 23537 | piperidin-1-yl | H | CH₂ | NHCO | CH₂ | 3-hydroxyphenyl |
| 23538 | piperidin-1-yl | H | CH₂ | NHCO | CH₂ | 4-hydroxyphenyl |
| 23552 | piperidin-1-yl | H | CH₂ | NHCO | CH₂ | 3-fluoro-4- |
| | | | | | | hydroxyphenyl |
| 23553 | piperidin-1-yl | H | CH₂ | NHCO | CH₂ | 3-methylisoxazol-5-yl |
| 23554 | piperidin-1-yl | H | CH₂ | NHCO | CHCOOH | phenyl |
| 23702 | 4-(cyclohexyl- | H | CH₂ | NHCO | CHF | phenyl |
| | carbonyloxy)-piperidin- | | | | | |
| | 1-yl | | | | | |
| 23703 | 4-(acetyloxy)-piperidin- | H | CH₂ | NHCO | CHF | phenyl |
| | 1-yl | | | | | |
| 23730 | 3-hydroxypiperidin-1- | H | CH₂ | NHCO | CF₂ | thien-3-yl |
| | yl | | | | | |
| 23760 | piperidin-1-yl | H | CH₂ | NHCO | CHNH₂ | phenyl |
| 23762 | 3-fluoro-4-hydroxy- | H | CH₂ | NHCO | CH₂ | phenyl |
| | piperidin-1-yl | | | | | |
| 23775 | 3-hydroxy- | H | CH₂ | NHCO | CF₂ | thien-2-yl |
| | (homopiperidin-1-yl) | | | | | |
| 23777 | 3,3-difluoropiperidin-1- | H | CH₂ | NHCO | CHCOCH | phenyl |
| | yl | | | | | |
| 23870 | 3-fluoro-4- | H | CH₂ | NHCO | CF₂ | thien-3-yl |
| | hydroxypiperidin-1-yl | | | | | |
| 23873 | 3-fluoro-4-hydroxy- | H | CH₂ | NHCO | CHCOOH | thien-3-yl |
| | piperidin-1-yl | | | | | |
| 23953 | 4-hydroxy- | H | CH₂ | NHCO | CF₂ | thien-2-yl |
| | (homopiperidin-1-yl) | | | | | |
| 24118 | 4-hydroxypiperidin-1- | H | CH₂ | NHCO | CH₂CH₂ | 4-methoxyphenyl |
| | yl | | | | | |
| 24119 | 4-hydroxypiperidin-1- | H | CH₂ | NHCO | CH₂CH₂ | 4-methylphenyl |
| | yl | | | | | |
| 24120 | 4-hydroxypiperidin-1- | H | CH₂ | NHCO | CH₂CH₂ | 3,4-difluorophenyl |
| | yl | | | | | |
| 24425 | piperidin-1-yl | H | CH₂ | NHCO | CH(CH₂OH) | phenyl |
| 24473 | trans-3,4-dihydroxy- | H | CH₂ | NHCO | CH₂ | phenyl |
| | piperidin-1-yl | | | | | |
| 24475 | trans-3,4-dihydroxy- | H | CH₂ | NHCO | CH₂ | thien-3-yl |
| | piperidin-1-yl | | | | | |
| 24476 | trans-3,4-dihydroxy- | H | CH₂ | NHCO | CH₂ | thien-2-yl |
| | piperidin-1-yl | | | | | |
| 24477 | trans-3,4-dihydroxy- | H | CH₂ | NHCO | CH₂ | 4-azidophenyl |
| | piperidin-1-yl | | | | | |
| 24478 | trans-3,4-dihydroxy- | H | CH₂ | NHCO | CH₂ | 3-azidophenyl |
| | piperidin-1-yl | | | | | |
| 24524 | 3-aza-bicyclo[3.1.0]- | H | CH₂ | NHCO | CH₂ | phenyl |
| | hexan-3-yl | | | | | |
| 24577 | 3-oxopiperazin-1-yl | H | CH₂ | NHCO | CH₂ | phenyl |
| 24578 | [1,4]oxazepan-4-yl | H | CH₂ | NHCO | CH₂ | phenyl |
| 24723 | piperidin-1-yl | H | CH₂ | NHCO | CH(C(O)OCH₂CH₃) | phenyl |
| 24725 | piperidin-1-yl | H | CH₂ | NHCO | CH₃NH(CO)CH | phenyl |
| 24726 | piperidin-1-yl | H | CH₂ | NHCO | (CH₃)₂N(CO)CH | phenyl |
| 24730 | 2-trifluoromethyl- | H | CH₂ | NHCO | CH₂ | phenyl |
| | pyrrolidin-1-yl | | | | | |
| 24731 | 4-trifluoromethyl- | H | CH₂ | NHCO | CH₂ | phenyl |
| | piperidin-1-yl | | | | | |
| 24732 | 3-oxopiperazin-1-yl | H | CH₂ | NHCO | CH₂ | thien-3-yl |
| 24733 | [1,4]oxazepan-4-yl | H | CH₂ | NHCO | CH₂ | thien-3-yl |
| 24734 | 2-trifluoromethyl- | H | CH₂ | NHCO | CH₂ | thien-3-yl |
| | pyrrolidin-1-yl | | | | | |
| 24735 | 4-trifluoromethyl- | H | CH₂ | NHCO | CH₂ | thien-3-yl |
| | piperidin-1-yl | | | | | |
| 24759 | 3-aza-bicyclo[3.1.0]- | H | CH₂ | NHCO | CH₂ | thien-3-yl |
| | hexan-3-yl | | | | | |
| 24760 | cis-3 -hydroxy-4- | H | CH₂ | NHCO | CH₂ | phenyl |
| | hydroxymethyl- | | | | | |
| | piperidin-1-yl | | | | | |
| 24761 | trans-3-hydroxy-4- | H | CH₂ | NHCO | CH₂ | phenyl |
| | hydroxymethyl- | | | | | |
| | pipendin-1-yl | | | | | |
| 24762 | cis-4-hydroxy-3- | H | CH₂ | NHCO | CH₂ | phenyl |
| | hydroxymethyl- | | | | | |
| | piperidin-1-yl | | | | | |
| 24763 | cis-3-hydroxy-4- | H | CH₂ | NHCO | CH₂ | thien-3-yl |
| | hydroxymethyl- | | | | | |
| | piperidin-1-yl | | | | | |
| 24764 | trans-3-hydroxy-4- | H | CH₂ | NHCO | CH₂ | thien-3-yl |
| | hydroxymethyl- | | | | | |
| | piperidin-1-yl | | | | | |
| 24765 | cis-4-hydroxy-3- | H | CH₂ | NHCO | CH₂ | thien-3-yl |
| | hydroxymethyl- | | | | | |
| | piperidin-1-yl | | | | | |
| 24929 | 8-oxa-3-aza- | H | CH₂ | NHCO | CH₂ | phenyl |
| | bicyclo[4.2.0]octan-3- | | | | | |
| | yl | | | | | |
| 24930 | 7-oxa-3-aza- | H | CH₂ | NHCO | CH₂ | phenyl |
| | bicyclo[4.2.0]octan-3- | | | | | |
| | yl | | | | | |
| 24931 | 4-acetylhomopiperazin- | H | CH₂ | NHCO | CH₂ | phenyl |
| | 1-yl | | | | | |
| 24933 | 8-oxa-3-aza- | H | CH₂ | NHCO | CH₂ | thien-3-yl |
| | bicyclo[4.2.0]octan-3- | | | | | |
| | yl | | | | | |
| 24934 | 7-oxa-3-aza- | H | CH₂ | NHCO | CH₂ | thien-3-yl |
| | bicyclo[4.2.0]octan-3- | | | | | |
| | yl | | | | | |
| 24935 | 4-acetylhomopiperazin- | H | CH₂ | NHCO | CH₂ | thien-3-yl |
| | 1-yl | | | | | |

are named as:
2-phenyl-N-{4-[4-(3,3-difluoropiperidin-1-ylcarbonyl)-oxazol-2-yl]-benzyl}-acetamide;
2-(4-trifluoromethoxyphenyl)-N-{4-[4-(piperidin-1-ylcarbonyl)-oxazol-2-yl]-benzyl}-acetamide;
2-fluoro-2-phenyl-N-{4-[4-(4-hydroxypiperidin-1-ylcarbonyl)-oxazol-2-yl]-benzyl}-acetamide;
2-phenyl-N-{4-[4-(3-hydroxypiperidin-1-ylcarbonyl)-oxazol-2-yl]-benzyl}-acetamide;
2-(4-chlorophenyl)-N-{4-[4-(piperidin-1-ylcarbonyl)-oxazol-2-yl]-benzyl}-acetamide;
2-(thien-3-yl)-N-{4-[4-(3-hydroxypiperidin-1-ylcarbonyl)-oxazol-2-yl]-benzyl}-acetamide;
2-(thien-3-yl)-N-{4-[4-(3,3-difluoropiperidin-1-ylcarbonyl)-oxazol-2-yl]-benzyl}-acetamide;
2-(thien-3-yl)-N-{4-[4-(homopiperidin-1-ylcarbonyl)-oxazol-2-yl]-benzyl}-acetamide;
2-phenyl-N-{4-[4-(piperidin-1-ylcarbonyl)-oxazol-2-yl]-benzyl}-acetamide;
2-fluoro-2-phenyl-N-{4-[4-(piperidin-1-ylcarbonyl)-oxazol-2-yl]-benzyl}-acetamide;
2-(thien-3-yl)-N-{4-[4-(piperidin-1-ylcarbonyl)-oxazol-2-yl]-benzyl}-acetamide;
2,2-difluoro-2-(thien-3-yl)-N-{4-[4-(piperidin-1-ylcarbonyl)-oxazol-2-yl]-benzyl}-acetamide;
2-phenyl-N-{4-[4-(homopiperidin-1-ylcarbonyl)-oxazol-2-yl]-benzyl}-acetamide;
2,2-difluoro-2-(thien-2-yl)-N-{4-[4-(4-hydroxyhomopiperidin-1-ylcarbonyl)-oxazol-2-yl]-benzyl}-acetamide;
2,2-difluoro-2-(thien-2-yl)-N-{4-[4-(3-hydroxypiperidin-1-ylcarbonyl)-oxazol-2-yl]-benzyl}-acetamide;
2-(thien-3-yl)-N-{4-[4-(4-hydroxyhomopiperidin-1-ylcarbonyl)-oxazol-2-yl]-benzyl}-acetamide;
2-(thien-3-yl)-N-{4-[4-(3,3-difluoro-4-hydroxypiperidin-1-ylcarbonyl)-oxazol-2-yl]-benzyl}-acetamide;
2-fluoro-2-(2-fluorophenyl)-N-{4-[4-(piperidin-1-ylcarbonyl)-oxazol-2-yl]-benzyl}-acetamide;
3-(2-methoxyphenyl)-N-{4-[4-(piperidin-1-ylcarbonyl)-oxazol-2-yl]-benzyl}-propionamide;
3-(3-methoxyphenyl)-N-{4-[4-(piperidin-1-ylcarbonyl)-oxazol-2-yl]-benzyl}-propionamide;
3-(4-methoxyphenyl)-N-{4-[4-(piperidin-1-ylcarbonyl)-oxazol-2-yl]-benzyl}-propionamide;
3-(4-methylphenyl)-N-{4-[4-(piperidin-1-ylcarbonyl)-oxazol-2-yl]-benzyl}-propionamide;
3-(3,4-difluorophenyl)-N-{4-[4-(piperidin-1-ylcarbonyl)-oxazol-2-yl]-benzyl}-propionamide;
3-[2,5-bis-(trifluoromethyl)phenyl]-N-{4-[4-(piperidin-1-ylcarbonyl)-oxazol-2-yl]-benzyl}-propionamide;
3-(3-fluorophenyl)-N-{4-[4-(piperidin-1-ylcarbonyl)-oxazol-2-yl]-benzyl}-propionamide;
3-(2-fluorophenyl)-N-{4-[4-(piperidin-1-ylcarbonyl)-oxazol-2-yl]-benzyl}-propionamide;
3-(3,4-methylenedioxyphenyl)-N-{4-[4-(piperidin-1-ylcarbonyl)-oxazol-2-yl]-benzyl}-propionamide;
3-(3,4-dichlorophenyl)-N-{4-[4-(piperidin-1-ylcarbonyl)-oxazol-2-yl]-benzyl}-propionamide;
3-(2,6-dichlorophenyl)-N-{4-[4-(piperidin-1-ylcarbonyl)-oxazol-2-yl]-benzyl}-propionamide;
3-(3-methylphenyl)-N-{4-[4-(piperidin-1-ylcarbonyl)-oxazol-2-yl]-benzyl}-propionamide;
3-(4-fluorophenyl)-N-{4-[4-(piperidin-1-ylcarbonyl)-oxazol-2-yl]-benzyl}-propionamide;
3-(2,4-dichlorophenyl)-N-{4-[4-(piperidin-1-ylcarbonyl)-oxazol-2-yl]-benzyl}-propionamide;
3-(2,5-dimethoxyphenyl)-N-{4-[4-(piperidin-1-ylcarbonyl)-oxazol-2-yl]-benzyl}-propionamide;
2-methyl-3-(phenyl)-N-{4-[4-(piperidin-1-ylcarbonyl)-oxazol-2-yl]-benzyl}-propionamide;
3-methyl-3-(phenyl)-N-{4-[4-(piperidin-1-ylcarbonyl)-oxazol-2-yl]-benzyl}-propionamide;
3-(2-methylphenyl)-N-{4-[4-(piperidin-1-ylcarbonyl)-oxazol-2-yl]-benzyl}-propionamide;
2-(3,4-methylenedioxyphenyl)-N-{4-[4-(piperidin-1-ylcarbonyl)-oxazol-2-yl]-benzyl}-acetamide;
2-(4-methoxy-3-methylphenyl)-N-{4-[4-(piperidin-1-ylcarbonyl)-oxazol-2-yl]-benzyl}-acetamide;
2-(3,4,5-trimethoxyphenyl)-N-{4-[4-(piperidin-1-ylcarbonyl)-oxazol-2-yl]-benzyl}-acetamide;
2-(4-methylphenyl)-N-{4-[4-(piperidin-1-ylcarbonyl)-oxazol-2-yl]-benzyl}-acetamide;
2-(pyridin-2-yl)-N-{4-[4-(piperidin-1-ylcarbonyl)-oxazol-2-yl]-benzyl}-acetamide;
2-(3,4-dimethoxyphenyl)-N-{4-[4-(piperidin-1-ylcarbonyl)-oxazol-2-yl]-benzyl}-acetamide;
3-(pyridin-2-yl)-N-{4-[4-(piperidin-1-ylcarbonyl)-oxazol-2-yl]-benzyl}-propionamide;
2-(4-methoxyphenyl)-N-{4-[4-(2-methylpiperidin-1-ylcarbonyl)-oxazol-2-yl]-benzyl}-acetamide;
3-(phenyl)-N-{4-[4-(2-methylpiperidin-1-ylcarbonyl)-oxazol-2-yl]-benzyl}-propionamide;
2-(4-ethoxyphenyl)-N-{4-[4-(piperidin-1-ylcarbonyl)-oxazol-2-yl]-benzyl}-acetamide;
2-(4-methoxyphenyl)-N-{4-[4-(3-methylpiperidin-1-ylcarbonyl)-oxazol-2-yl]-benzyl}-acetamide;
3-(furan-2-yl)-N-{4-[4-(piperidin-1-ylcarbonyl)-oxazol-2-yl]-benzyl}-propionamide;
3-(phenyl)-N-{4-[4-(3-methylpiperidin-1-ylcarbonyl)-oxazol-2-yl]-benzyl}-propionamide;
4-(thien-2-yl)-N-{4-[4-(piperidin-1-ylcarbonyl)-oxazol-2-yl]-benzyl}-butyramide;
2-(pyridin-3-yl)-N-{4-[4-(piperidin-1-ylcarbonyl)-oxazol-2-yl]-benzyl}-acetamide;
2-(3,5-dimethylphenyl)-N-{4-[4-(piperidin-1-ylcarbonyl)-oxazol-2-yl]-benzyl}-acetamide;
3-(thien-2-yl)-N-{4-[4-(piperidin-1-ylcarbonyl)-oxazol-2-yl]-benzyl}-propionamide;
2-(thien-2-yl)-N-{4-[4-(3-methylpiperidin-1-ylcarbonyl)-oxazol-2-yl]-benzyl}-acetamide;
2-(thien-2-yl)-N-{4-[4-(thiomorpholin-4-ylcarbonyl)-oxazol-2-yl]-benzyl}-acetamide;
2-(thien-2-yl)-N-{4-[4-(1,4-dioxa-8-aza-spiro[4.5]decan-8-ylcarbonyl)-oxazol-2-yl]-benzyl}-acetamide;
2-(thien-2-yl)-N-{4-[4-(2,6-dimethylpiperidin-1-ylcarbonyl)-oxazol-2-yl]-benzyl}-acetamide;
2-(thien-2-yl)-N-{4-[4-(2-methylpiperidin-1-ylcarbonyl)-oxazol-2-yl]-benzyl}-acetamide;
3-(phenyl)-N-{4-[4-(3,5-dimethylpiperidin-1-ylcarbonyl)-oxazol-2-yl]-benzyl}-propionamide;
3-(phenyl)-N-{4-[4-(4-hydroxymethylpiperidin-1-ylcarbonyl)-oxazol-2-yl]-benzyl}-propionamide;
2-(4-methoxyphenyl)-N-{4-[4-(4-hydroxypiperidin-1-ylcarbonyl)-oxazol-2-yl]-benzyl}-acetamide;
2,2-dimethyl-N-methyl-2-phenyl-N-{4-[4-(piperidin-1-ylcarbonyl)-oxazol-2-yl]-benzyl}-acetamide;
N-methyl-2-phenyl-N-{4-[4-(piperidin-1-ylcarbonyl)-oxazol-2-yl]-benzyl}-acetamide;
2-(4-methoxyphenyl)-N-{4-[4-(thiomorpholin-4-ylcarbonyl)-oxazol-2-yl]-benzyl}-acetamide;
2-(4-methoxyphenyl)-N-{4-[4-(1,2,3,6-tetrahydro-pyridin-1-ylcarbonyl)-oxazol-2-yl]-benzyl}-acetamide;
2,2-difluoro-2-phenyl-N-{4-[4-(piperidin-1-ylcarbonyl)-oxazol-2-yl]-benzyl}-acetamide;
2-(thien-2-yl)-N-{4-[4-(pyrrolidin-1-ylcarbonyl)-oxazol-2-yl]-benzyl}-acetamide;
2-(thien-2-yl)-N-{4-[4-(4-bromopiperidin-1-ylcarbonyl)-oxazol-2-yl]-benzyl}-acetamide;
2-(thien-2-yl)-N-{4-[4-(1,2,3,6-tetrahydro-pyridin-1-ylcarbonyl)-oxazol-2-yl]-benzyl}-acetamide;
2-(4-methoxyphenyl)-N-{4-[4-(homopiperidin-1-ylcarbonyl)-oxazol-2-yl]-benzyl}-acetamide;
2-(4-methoxyphenyl)-N-{4-[4-(4-hydroxymethylpiperidin-1-ylcarbonyl)-oxazol-2-yl]-benzyl}-acetamide;
3-phenyl-N-{4-[4-(2,6-dimethylpiperidin-1-ylcarbonyl)-oxazol-2-yl]-benzyl}-propionamide;
2-phenyl-N-{4-[5-methyl-4-(piperidin-1-ylcarbonyl)-oxazol-2-yl]-benzyl}-acetamide;
2-(thien-2-yl)-N-{4-[5-methyl-4-(piperidin-1-ylcarbonyl)-oxazol-2-yl]-benzyl}-acetamide;
2-(4-methoxyphenyl)-N-{4-[4-(2,6-dimethylpiperidin-1-ylcarbonyl)-oxazol-2-yl]-benzyl}-acetamide;
2-phenyl-N-{4-[4-(4-methylpiperidin-1-ylcarbonyl)-oxazol-2-yl]-benzyl}-acetamide;
2-phenyl-N-{4-[4-(2-methylpiperidin-1-ylcarbonyl)-oxazol-2-yl]-benzyl}-acetamide;
2-phenyl-N-{4-[4-(4-hydroxypiperidin-1-ylcarbonyl)-oxazol-2-yl]-benzyl}-acetamide;
2-phenyl-N-{4-[4-(thiomorpholin-4-ylcarbonyl)-oxazol-2-yl]-benzyl}-acetamide;
2-phenyl-N-{4-[4-(3-methylpiperidin-1-ylcarbonyl)-oxazol-2-yl]-benzyl}-acetamide;
2-phenyl-N-{4-[4-(morpholin-4-ylcarbonyl)-oxazol-2-yl]-benzyl}-acetamide;
2-phenyl-N-{4-[4-(4-hydroxypiperidin-1-ylcarbonyl)-oxazol-2-yl]-benzyl}-acetamide;
2-phenyl-N-{4-[4-(4-bromopiperidin-1-ylcarbonyl)-oxazol-2-yl]-benzyl}-acetamide;
2-phenyl-N-{4-[4-(1,2,3,6-tetrahydro-pyridin-1-ylcarbonyl)-oxazol-2-yl]-benzyl}-acetamide;
2-phenyl-N-{4-[4-(pyrrolidin-1-ylcarbonyl)-oxazol-2-yl]-benzyl}-acetamide;
2-phenyl-N-{4-[5-methyl-4-(2-methylpiperidin-1-ylcarbonyl)-oxazol-2-yl]-benzyl}-acetamide;
2-(thien-2-yl)-N-{4-[5-methyl-4-(2-methylpiperidin-1-ylcarbonyl)-oxazol-2-yl]-benzyl}-acetamide;
2-(4-methoxyphenyl)-N-{4-[4-(2,6-dimethylpiperidin-1-ylcarbonyl)-oxazol-2-yl]-benzyl}-acetamide;
2,2-difluoro-2-phenyl-N-{4-[4-(homopiperidin-1-ylcarbonyl)-oxazol-2-yl]-benzyl}-acetamide;
2,2-difluoro-2-phenyl-N-{4-[4-(1,2,3,6-tetrahydro-pyridin-1-ylcarbonyl)-oxazol-2-yl]-benzyl}-acetamide;
2-fluoro-2-phenyl-N-{4-[4-(homopiperidin-1-ylcarbonyl)-oxazol-2-yl]-benzyl}-acetamide;
2-fluoro-2-phenyl-N-{4-[4-(1,2,3,6-tetrahydro-pyridin-1-ylcarbonyl)-oxazol-2-yl]-benzyl}-acetamide;
2-fluoro-2-phenyl-N-{4-[4-(pyrrolidin-1-ylcarbonyl)-oxazol-2-yl]-benzyl}-acetamide;
2-(2-fluorophenyl)-N-{4-[4-(piperidin-1-ylcarbonyl)-oxazol-2-yl]-benzyl}-acetamide;
2-(3-fluorophenyl)-N-{4-[4-(piperidin-1-ylcarbonyl)-oxazol-2-yl]-benzyl}-acetamide;
2-(4-fluorophenyl)-N-{4-[4-(piperidin-1-ylcarbonyl)-oxazol-2-yl]-benzyl}-acetamide;
2-(2,6-difluorophenyl)-N-{4-[4-(piperidin-1-ylcarbonyl)-oxazol-2-yl]-benzyl}-acetamide;
2-(3-chlorophenyl)-N-{4-[4-(piperidin-1-ylcarbonyl)-oxazol-2-yl]-benzyl}-acetamide;
2-phenyl-N-{4-[4-(piperidin-1-ylcarbonyl)-oxazol-2-yl]-phenethyl}-acetamide;
2-(furan-2-yl)-N-{4-[4-(piperidin-1-ylcarbonyl)-oxazol-2-yl]-benzyl}-acetamide;
2-(4-trifluoromethoxyphenyl)-N-{4-[4-(3-hydroxypiperidin-1-ylcarbonyl)-oxazol-2-yl]-benzyl}-acetamide;
2-(4-trifluoromethoxyphenyl)-N-{4-[4-(4-hydroxypiperidin-1-ylcarbonyl)-oxazol-2-yl]-benzyl}-acetamide;
2-phenyl-N-{4-[5-bromo-4-(piperidin-1-ylcarbonyl)-oxazol-2-yl]-benzyl}-acetamide;
2-phenyl-N-{4-[4-(azetidin-1-ylcarbonyl)-oxazol-2-yl]-benzyl}-acetamide;
2-phenyl-N-{4-[4-(2-methylpyrrolidin-1-ylcarbonyl)-oxazol-2-yl]-benzyl}-acetamide;
2-phenyl-N-{4-[4-(3-hydroxypyrrolidin-1-ylcarbonyl)-oxazol-2-yl]-benzyl}-acetamide;
2-phenyl-N-{4-[4-(2,5-dimethylpyrrolidin-1-ylcarbonyl)-oxazol-2-yl]-benzyl}-acetamide;
2-phenyl-N-{4-[4-(trans-2,5-dimethyl-2,5-dihydro-1H-pyrrol-1-ylcarbonyl)-oxazol-2-yl]-benzyl}-acetamide;
2-phenyl-N-{4-[4-(thiazolidin-3-ylcarbonyl)-oxazol-2-yl]-benzyl}-acetamide;
2-phenyl-N-{4-[4-(2-methylthiazolidin-3-ylcarbonyl)-oxazol-2-yl]-benzyl}-acetamide;
2-phenyl-N-{4-[4-(3,3-dimethylpiperidin-1-ylcarbonyl)-oxazol-2-yl]-benzyl}-acetamide;
2-phenyl-N-{4-[4-(piperazin-1-ylcarbonyl)-oxazol-2-yl]-benzyl}-acetamide;
2-phenyl-N-{4-[4-(4-acetylpiperazin-1-ylcarbonyl)-oxazol-2-yl]-benzyl}-acetamide;
2-phenyl-N-{4-[4-(1-oxothiomorpholin-4-ylcarbonyl)-oxazol-2-yl]-benzyl}-acetamide;
2-phenyl-N-{4-[4-(1,1-dioxothiomorpholin-4-ylcarbonyl)-oxazol-2-yl]-benzyl}-acetamide;
N-methyl-2-phenyl-N-{4-[4-(3-hydroxypiperidin-1-ylcarbonyl)-oxazol-2-yl]-benzyl}-acetamide;
2-phenyl-N-{4-[4-(3-methoxypiperidin-1-ylcarbonyl)-oxazol-2-yl]-benzyl}-acetamide;
N-methyl-2-phenyl-N-{4-[4-(3-methoxypiperidin-1-yl carbonyl)-oxazol-2-yl]-benzyl}-acetamide;
N-methyl-2-phenyl-N-{4-[4-(4-hydroxypiperidin-1-ylcarbonyl)-oxazol-2-yl]-benzyl}-acetamide;
2-phenyl-N-{4-[4-(4-methoxypiperidin-1-ylcarbonyl)-oxazol-2-yl]-benzyl}-acetamide;
N-methyl-2-phenyl-N-{4-[4-(4-methoxypiperidin-1-ylcarbonyl)-oxazol-2-yl]-benzyl}-acetamide;
2-phenyl-N-{4-[4-(homopiperazin-1-ylcarbonyl)-oxazol-2-yl]-benzyl}-acetamide;
2-phenyl-N-{4-[4-(4-methylhomopiperazin-1-ylcarbonyl)-oxazol-2-yl]-benzyl}-acetamide;
2-phenyl-N-{4-[4-(azocan-1-ylcarbonyl)-oxazol-2-yl]-benzyl}-acetamide;
2-phenyl-N-{4-[4-(1,2,3,4-tetrahydro-isoquinolin-2-ylcarbonyl)-oxazol-2-yl]-benzyl}-acetamide;
2-phenyl-N-{4-[4-(decahydroisoquinolin-2-ylcarbonyl)-oxazol-2-yl]-benzyl}-acetamide;
2-phenyl-N-{4-[4-(3-aza-bicyclo[2.2.1]hept-5-en-3-ylcarbonyl)-oxazol-2-yl]-benzyl}-acetamide;
2-phenyl-N-{4-[4-(3-aza-bicyclo[3.2.2]non-6-ene-3-ylcarbonyl)-oxazol-2-yl]-benzyl}-acetamide;
2-phenyl-N-{4-[4-(4-fluoropiperidin-1-ylcarbonyl)-oxazol-2-yl]-benzyl}-acetamide;
2-phenyl-N-{4-[4-(4,4-difluoropiperidin-1-ylcarbonyl)-oxazol-2-yl]-benzyl}-acetamide;
2-phenyl-N-{4-[4-(2-methylaziridin-1-ylcarbonyl)-oxazol-2-yl]-benzyl}-acetamide;
2-(4-trifluoromethoxyphenyl)-N-{4-[4-(4-hydroxymethylpiperidin-1-ylcarbonyl)-oxazol-2-yl]-benzyl}-acetamide;
2-fluoro-2-phenyl-N-{4-[4-(3-hydroxypiperidin-1-ylcarbonyl)-oxazol-2-yl]-benzyl}-acetamide;
2-fluoro-2-phenyl-N-{4-[4-(4-hydroxymethylpiperidin-1-ylcarbonyl)-oxazol-2-yl]-benzyl}-acetamide;
2-(thien-3-yl)-N-{4-[4-(4-hydroxypiperidin-1-ylcarbonyl)-oxazol-2-yl]-benzyl}-acetamide;
2-(furan-2-yl)-N-{4-[4-(4-hydroxypiperidin-1-ylcarbonyl)-oxazol-2-yl]-benzyl}-acetamide;
2-(furan-2-yl)-N-{4-[4-(3-hydroxypiperidin-1-ylcarbonyl)-oxazol-2-yl]-benzyl}-acetamide;
2-(furan-2-yl)-N-{4-[4-(4-hydroxymethylpiperidin-1-ylcarbonyl)-oxazol-2-yl]-benzyl}-acetamide;
2-phenyl-N-{4-[4-(3-fluoropiperidin-1-ylcarbonyl)-oxazol-2-yl]-benzyl}-acetamide;
2-phenyl-N-{4-[4-(4-oxopiperidin-1-ylcarbonyl)-oxazol-2-yl]-benzyl}-acetamide;
2-phenyl-N-{4-[4-(trans-2,5-dimethylpiperazin-1-ylcarbonyl)-oxazol-2-yl]-benzyl}-acetamide;
2-phenyl-N-{4-[4-(3-oxopiperidin-1-ylcarbonyl)-oxazol-2-yl]-benzyl}-acetamide;
2-phenyl-N-{4-[4-(4-chloropiperidin-1-ylcarbonyl)-oxazol-2-yl]-benzyl}-acetamide;
2-phenyl-N-{4-[4-(3-chloropiperidin-1-ylcarbonyl)-oxazol-2-yl]-benzyl}-acetamide;
2-fluoro-2-phenyl-N-{4-[4-(4-fluoropiperidin-1-ylcarbonyl)-oxazol-2-yl]-benzyl}-acetamide;
2-fluoro-2-phenyl-N-{4-[4-(4-methoxycarbonylpiperidin-1-ylcarbonyl)-oxazol-2-yl]-benzyl}-acetamide;
2-fluoro-2-phenyl-N-{4-[4-(4-carboxypiperidin-1-ylcarbonyl)-oxazol-2-yl]-benzyl}-acetamide;
1-phenyl-N-{4-[4-(piperidin-1-ylcarbonyl)-oxazol-2-yl]-benzyl}-methanesulfonamide;
2-fluoro-2-phenyl-N-{4-[4-(4-ethoxycarbonylpiperidin-1-ylcarbonyl)-oxazol-2-yl]-benzyl}-acetamide;
2-phenyl-N-{4-[4-(2-methoxycarbonylpiperidin-1-ylcarbonyl)-oxazol-2-yl]-benzyl}-acetamide;
2-fluoro-2-phenyl-N-{4-[4-(3,3-difluoropiperidin-1-ylcarbonyl)-oxazol-2-yl]-benzyl}-acetamide;
2-fluoro-2-phenyl-N-{4-[4-(azocan-1-ylcarbonyl)-oxazol-2-yl]-benzyl}-acetamide;
2-fluoro-2-phenyl-N-{4-[4-(2-methylpyrrolidin-1-ylcarbonyl)-oxazol-2-yl]-benzyl}-acetamide;
2-(4-trifluoromethoxyphenyl)-N-{4-[4-(morpholin-4-ylcarbonyl)-oxazol-2-yl]-benzyl}-acetamide;
2-fluoro-2-phenyl-N-{4-[4-(morpholin-4-ylcarbonyl)-oxazol-2-yl]-benzyl}-acetamide;
2-(thien-3-yl)-N-{4-[4-(morpholin-4-ylcarbonyl)-oxazol-2-yl]-benzyl}-acetamide;
2-fluoro-2-phenyl-N-{4-[4-(2,5-dimethylpyrrolidin-1-ylcarbonyl)-oxazol-2-yl]-benzyl}-acetamide;
2-phenyl-N-{4-[4-(2S-methoxycarbonylpyrrolidin-1-ylcarbonyl)-oxazol-2-yl]-benzyl}-acetamide;
2-phenyl-N-{4-[4-(2S-hydroxymethylpyrrolidin-1-ylcarbonyl)-oxazol-2-yl]-benzyl}-acetamide;
2-phenyl-N-{4-[4-(2R-hydroxymethylpyrrolidin-1-ylcarbonyl)-oxazol-2-yl]-benzyl}-acetamide;
2-phenyl-N-{4-[4-(trans-2,5-dimethylpyrrolidin-1-ylcarbonyl)-oxazol-2-yl]-benzyl}-acetamide;
2-(thien-3-yl)-N-{4-[4-(2,5-dimethylpyrrolidin-1-ylcarbonyl)-oxazol-2-yl]-benzyl}-acetamide;
2-(thien-3-yl)-N-{4-[4-(1,2,3,6-tetrahydro-pyridin-1-ylcarbonyl)-oxazol-2-yl]-benzyl}-acetamide;
2-(thien-3-yl)-N-{4-[4-(3-methylpiperidin-1-ylcarbonyl)-oxazol-2-yl]-benzyl}-acetamide;
2-(thien-3-yl)-N-{4-[4-(2-methylpyrrolidin-1-ylcarbonyl)-oxazol-2-yl]-benzyl}-acetamide;
2-phenyl-N-{4-[4-(cis-3,4-dihydroxypiperidin-1-ylcarbonyl)-oxazol-2-yl]-benzyl}-acetamide;
2-(thien-3-yl)-N-{4-[4-(3-chloropiperidin-1-ylcarbonyl)-oxazol-2-yl]-benzyl}-acetamide;
2-(thien-3-yl)-N-{4-[4-(4-chloropiperidin-1-ylcarbonyl)-oxazol-2-yl]-benzyl}-acetamide;
2-(thien-3-yl)-N-{4-[4-(3,5-dimethylpiperidin-1-ylcarbonyl)-oxazol-2-yl]-benzyl}-acetamide;
2-(thien-3-yl)-N-{4-{4-[2-(2-hydroxyethyl)piperidin-1-ylcarbonyl]-oxazol-2-yl}-benzyl}-acetamide;
2-(thien-3-yl)-N-{4-[4-(2,6-dimethylpiperidin-1-ylcarbonyl)-oxazol-2-yl]-benzyl}-acetamide;
2-(thien-3-yl)-N-{4-[4-(4,4-difluoropiperidin-1-ylcarbonyl)-oxazol-2-yl]-benzyl}-acetamide;
2-(thien-3-yl)-N-{4-[4-(4-fluoropiperidin-1-ylcarbonyl)-oxazol-2-yl]-benzyl}-acetamide;
2-phenyl-N-{4-[4-(3,4-difluoropiperidin-1-ylcarbonyl)-oxazol-2-yl]-benzyl}-acetamide;
sulfuric acid mono-(3-hydroxy-1-{2-[4-(phenylacetylamino-methyl)-phenyl]-oxazol-4-ylcarbonyl}-piperidin-4-yl) ester (named by autonom);
2-(thien-3-yl)-N-{4-[4-(3-methoxypiperidin-1-ylcarbonyl)-oxazol-2-yl]-benzyl}-acetamide;
2-(thien-3-yl)-N-{4-[4-(3-hydroxymethylpiperidin-1-ylcarbonyl)-oxazol-2-yl]-benzyl}-acetamide;
2-(thien-3-yl)-N-{4-[4-(2-hydroxymethylpiperidin-1-ylcarbonyl)-oxazol-2-yl]-benzyl}-acetamide;
2-(thien-3-yl)-N-{4-[4-(thiomorpholin-4-ylcarbonyl)-oxazol-2-yl]-benzyl}-acetamide;
2-(thien-3-yl)-N-{4-[4-(azocan-1-ylcarbonyl)-oxazol-2-yl]-benzyl}-acetamide;
2-(thien-3-yl)-N-{4-[4-(4-methylpiperidin-1-ylcarbonyl)-oxazol-2-yl]-benzyl}-acetamide;
2-(thien-3-yl)-N-{4-[4-(3-fluoropiperidin-1-ylcarbonyl)-oxazol-2-yl]-benzyl}-acetamide;
2-phenyl-N-{4-[4-(3-hydroxy-(homopiperidin-1-yl)carbonylcarbonyl)-oxazol-2-yl]-benzyl}-acetamide;
2-phenyl-N-{4-[4-(4-hydroxy-(homopiperidin-1-yl)carbonylcarbonyl)-oxazol-2-yl]-benzyl}-acetamide;
2-phenyl-N-{4-[4-(3R-hydroxypiperidin-1-ylcarbonyl)-oxazol-2-yl]-benzyl}-acetamide;
2-(thien-3-yl)-N-{4-[4-(3-hydroxy-(homopiperidin-1-yl)carbonylcarbonyl)-oxazol-2-yl]-benzyl}-acetamide;
2-(thien-3-yl)-N-{4-[4-(3R-hydroxypiperidin-1-ylcarbonyl)-oxazol-2-yl]-benzyl}-acetamide;
2,2-difluoro-2-(thien-3-yl)-N-{4-[4-(4-hydroxypiperidin-1-ylcarbonyl)-oxazol-2-yl]-benzyl}-acetamide;
2-(thien-3-yl)-N-{4-[4-(3-trifluoromethylpiperidin-1-ylcarbonyl)-oxazol-2-yl]-benzyl}-acetamide;
2-phenyl-N-{4-[4-(3-trifluoromethylpiperidin-1-ylcarbonyl)-oxazol-2-yl]-benzyl}-acetamide;
2,2-difluoro-2-(thien-3-yl)-N-{4-[4-(3,3-difluoropiperidin-1-ylcarbonyl)-oxazol-2-yl]-benzyl}-acetamide;
2,2-difluoro-2-(thien-2-yl)-N-{4-[4-(3,3-difluoropiperidin-1-ylcarbonyl)-oxazol-2-yl]-benzyl}-acetamide;
2,2-difluoro-2-(thien-2-yl)-N-{4-[4-(piperidin-1-ylcarbonyl)-oxazol-2-yl]-benzyl}-acetamide;
2,2-difluoro-2-(thien-2-yl)-N-{4-[4-(4-hydroxypiperidin-1-ylcarbonyl)-oxazol-2-yl]-benzyl}-acetamide;
2,2-difluoro-2-phenyl-N-{4-[4-(4-hydroxypiperidin-1-ylcarbonyl)-oxazol-2-yl]-benzyl}-acetamide;
2,2-difluoro-2-phenyl-N-{4-[4-(3-hydroxypiperidin-1-ylcarbonyl)-oxazol-2-yl]-benzyl}-acetamide;
2,2-difluoro-2-phenyl-N-{4-[4-(3,3-difluoropiperidin-1-ylcarbonyl)-oxazol-2-yl]-benzyl}-acetamide;
2-phenyl-N-{4-[4-(trans-4-fluoro-3-hydroxy-(homopiperidin-1-yl)carbonyl)-oxazol-2-yl]-benzyl}-acetamide;
2-(thien-3-yl)-N-{4-[4-(trans-4-fluoro-3-hydroxy-(homopiperidin-1-yl)carbonyl)-oxazol-2-yl]-benzyl}-acetamide;
2-(thien-3-yl)-N-{4-[4-(3-fluoro-4-hydroxypiperidin-1-ylcarbonyl)-oxazol-2-yl]-benzyl}-acetamide;
2,2-difluoro-2-(pyridin-2-yl)-N-{4-[4-(piperidin-1-ylcarbonyl)-oxazol-2-yl]-benzyl}-acetamide;
2,2-difluoro-2-(pyridin-2-yl)-N-{4-[4-(4-hydroxypiperidin-1-ylcarbonyl)-oxazol-2-yl]-benzyl}-acetamide;
2-hydroxy-2-phenyl-N-{4-[4-(piperidin-1-ylcarbonyl)-oxazol-2-yl]-benzyl}-acetamide;
2-hydroxy-2-phenyl-N-{4-[4-(3,3-difluoropiperidin-1-ylcarbonyl)-oxazol-2-yl]-benzyl}-acetamide;
2R-hydroxy-2-phenyl-N-{4-[4-(3,3-difluoropiperidin-1-ylcarbonyl)-oxazol-2-yl]-benzyl}-acetamide;
2-fluoro-2-phenyl-N-{4-[4-(3-chloropiperidin-1-ylcarbonyl)-oxazol-2-yl]-benzyl}-acetamide;
2-(2-fluorophenyl)-N-{4-[4-(3,3-difluoropiperidin-1-ylcarbonyl)-oxazol-2-yl]-benzyl}-acetamide;
2-(3-fluorophenyl)-N-{4-[4-(3,3-difluoropiperidin-1-ylcarbonyl)-oxazol-2-yl]-benzyl}-acetamide;
2-(2-fluorophenyl)-N-{4-[4-(4-hydroxypiperidin-1-ylcarbonyl)-oxazol-2-yl]-benzyl}-acetamide;
2-(3-fluorophenyl)-N-{4-[4-(4-hydroxypiperidin-1-ylcarbonyl)-oxazol-2-yl]-benzyl}-acetamide;
2-(2-fluorophenyl)-N-{4-[4-(3-hydroxypiperidin-1-ylcarbonyl)-oxazol-2-yl]-benzyl}-acetamide;
2-(3-fluorophenyl)-N-{4-[4-(3-hydroxypiperidin-1-ylcarbonyl)-oxazol-2-yl]-benzyl}-acetamide;
2-fluoro-2-phenyl-N-{4-[4-(3-fluoropiperidin-1-ylcarbonyl)-oxazol-2-yl]-benzyl}-acetamide;
2S-hydroxy-2-phenyl-N-{4-[4-(3,3-difluoropiperidin-1-ylcarbonyl)-oxazol-2-yl]-benzyl}-acetamide;
2-(thien-3-yl)-N-{4-[4-([1,3]oxazinan-3-ylcarbonyl)-oxazol-2-yl]-benzyl}-acetamide;
2-phenyl-N-{4-[4-([1,3]oxazinan-3-ylcarbonyl)-oxazol-2-yl]-benzyl}-acetamide;
2-fluoro-2-(2-fluorophenyl)-N-{4-[4-(3-hydroxypiperidin-1-ylcarbonyl)-oxazol-2-yl]-benzyl}-acetamide;
2-fluoro-2-(2-fluorophenyl)-N-{4-[4-(4-hydroxypiperidin-1-ylcarbonyl)-oxazol-2-yl]-benzyl}-acetamide;
2-fluoro-2-(2-fluorophenyl)-N-{4-[4-(3,3-difluoropiperidin-1-ylcarbonyl)-oxazol-2-yl]-benzyl}-acetamide;
2-phenyl-N-{4-{4-[3-({[(CH₃)₃C]O(CO)NH}{[(CH₃)₃C]O(CO)CH₂}CH(CO)NH)-4-hydroxypiperidin-1-ylcarbonyl]-oxazol-2-yl}-benzyl}-acetamide;
2-(4-azidophenyl)-N-{4-[4-(piperidin-1-ylcarbonyl)-oxazol-2-yl]-benzyl}-acetamide;
2-(3-azidophenyl)-N-{4-[4-(piperidin-1-ylcarbonyl)-oxazol-2-yl]-benzyl}-acetamide;
2-(4-iodophenyl)-N-{4-[4-(trans-2,5-dimethylpyrrolidin-1-ylcarbonyl)-oxazol-2-yl]-benzyl}-acetamide;
2-(4-iodophenyl)-N-{4-[4-(cis-2,5-dimethylpyrrolidin-1-ylcarbonyl)-oxazol-2-yl]-benzyl}-acetamide;
2-(4-iodophenyl)-N-{4-[4-(trans-2,5-dimethyl-2,5-dihydro-1H-pyrrol-1-ylcarbonyl)-oxazol-2-yl]-benzyl}-acetamide;
2-(4-iodophenyl)-N-{4-[4-(cis-2,5-dimethyl-2,5-dihydro-1H-pyrrol-1-ylcarbonyl)-oxazol-2-yl]-benzyl}-acetamide;
2-phenyl-N-{4-[4-(cis-2,5-dimethyl-2,5-dihydro-1H-pyrrol-1-ylcarbonyl)-oxazol-2-yl]-benzyl}-acetamide;
2-(thien-3-yl)-N-{4-[4-(3-fluoro-4-oxopiperidin-1-ylcarbonyl)-oxazol-2-yl]-benzyl}-acetamide;
2-(2-hydroxyphenyl)-N-{4-[4-(piperidin-1-ylcarbonyl)-oxazol-2-yl]-benzyl}-acetamide;
2-(3-hydroxyphenyl)-N-{4-[4-(piperidin-1-ylcarbonyl)-oxazol-2-yl]-benzyl}-acetamide;
2-(4-hydroxyphenyl)-N-{4-[4-(piperidin-1-ylcarbonyl)-oxazol-2-yl]-benzyl}-acetamide;
2-(3-fluoro-4-hydroxyphenyl)-N-{4-[4-(piperidin-1-ylcarbonyl)-oxazol-2-yl]-benzyl}-acetamide;
2-(3-methylisoxazol-5-yl)-N-{4-[4-(piperidin-1-ylcarbonyl)-oxazol-2-yl]-benzyl}-acetamide;
2-carboxy-2-phenyl-N-{4-[4-(piperidin-1-ylcarbonyl)-oxazol-2-yl]-benzyl}-acetamide;
2-fluoro-2-phenyl-N-{4-{4-[4-(cyclohexylcarbonyloxy)-piperidin-1-ylcarbonyl]-oxazol-2-yl}-benzyl}-acetamide;
2-fluoro-2-phenyl-N-{4-{4-[4-(acetyloxy)piperidin-1-ylcarbonyl]-oxazol-2-yl}-benzyl}-acetamide
2,2-difluoro-2-(thien-3-yl)-N-{4-[4-(3-hydroxypiperidin-1-ylcarbonyl)-oxazol-2-yl]-benzyl}-acetamide
2-amino-2-phenyl-N-{4-[4-(piperidin-1-ylcarbonyl)-oxazol-2-yl]-benzyl}-acetamide;
2-phenyl-N-{4-[4-(3-fluoro-4-hydroxypiperidin-1-ylcarbonyl)-oxazol-2-yl]-benzyl}-acetamide;
2,2-difluoro-2-(thien-2-yl)-N-{4-[4-(3-hydroxy-(homopiperidin-1-yl)carbonyl)-oxazol-2-yl]-benzyl}-acetamide;
2-carboxy-2-phenyl-N-{4-[4-(3,3-difluoropiperidin-1-ylcarbonyl)-oxazol-2-yl]-benzyl}-acetamide;
2,2-difluoro-2-(thien-3-yl)-N-{4-[4-(3-fluoro-4-hydroxypiperidin-1-ylcarbonyl)-oxazol-2-yl]-benzyl}-acetamide;
2-carboxy-2-(thien-3-yl)-N-{4-[4-(3-fluoro-4-hydroxypiperidin-1-ylcarbonyl)-oxazol-2-yl]-benzyl}-acetamide;
2,2-difluoro-2-(thien-2-yl)-N-{4-[4-(4-hydroxy-(homopiperidin-1-yl)carbonyl)-oxazol-2-yl]-benzyl}-acetamide;
3-(4-methoxyphenyl)-N-{4-[4-(4-hydroxypiperidin-1-ylcarbonyl)-oxazol-2-yl]-benzyl}-propionamide;
3-(4-methylphenyl)-N-{4-[4-(4-hydroxypiperidin-1-ylcarbonyl)-oxazol-2-yl]-benzyl}-propionamide;
3-(3,4-difluorophenyl)-N-{4-[4-(4-hydroxypiperidin-1-ylcarbonyl)-oxazol-2-yl]-benzyl}-propionamide;
2-hydroxymethyl-2-phenyl-N-{4-[4-(piperidin-1-ylcarbonyl)-oxazol-2-yl]-benzyl}-acetamide;
2-phenyl-N-{4-[4-(trans-3,4-dihydroxypiperidin-1-ylcarbonyl)-oxazol-2-yl]-benzyl}-acetamide;
2-(thien-3-yl)-N-{4-[4-(trans-3,4-dihydroxypiperidin-1-ylcarbonyl)-oxazol-2-yl]-benzyl}-acetamide;
2-(thien-2-yl)-N-{4-[4-(trans-3,4-dihydroxypiperidin-1-ylcarbonyl)-oxazol-2-yl]-benzyl}-acetamide;
2-(4-azidophenyl)-N-{4-[4-(trans-3,4-dihydroxypiperidin-1-ylcarbonyl)-oxazol-2-yl]-benzyl}-acetamide;
2-(3-azidophenyl)-N-{4-[4-(trans-3,4-dihydroxypiperidin-1-ylcarbonyl)-oxazol-2-yl]-benzyl}-acetamide;
2-phenyl-N-{4-[4-(3-aza-bicyclo[3.1.0]hexan-3-ylcarbonyl)-oxazol-2-yl]-benzyl}-acetamide;
2-phenyl-N-{4-[4-(3-oxopiperazin-1-ylcarbonyl)-oxazol-2-yl]-benzyl}-acetamide;
2-phenyl-N-{4-[4-([1,4]oxazepan-4-ylcarbonyl)-oxazol-2-yl]-benzyl}-acetamide;
2-ethoxycarbonyl-2-phenyl-N-{4-[4-(piperidin-1-ylcarbonyl)-oxazol-2-yl]-benzyl}-acetamide;
2-(N-methylaminocarbonyl)-2-phenyl-N-{4-[4-(piperidin-1-ylcarbonyl)-oxazol-2-yl]-benzyl}-acetamide;
2-(N,N-dimethylaminocarbonyl)-2-phenyl-N-{4-[4-(piperidin-1-ylcarbonyl)-oxazol-2-yl]-benzyl}-acetamide;
2-phenyl-N-{4-[4-(2-trifluoromethylpyrrolidin-1-ylcarbonyl)-oxazol-2-yl]-benzyl}-acetamide;
2-phenyl-N-{4-[4-(4-trifluoromethylpiperidin-1-ylcarbonyl)-oxazol-2-yl]-benzyl}-acetamide;
2-(thien-3-yl)-N-{4-[4-(3-oxopiperazin-1-ylcarbonyl)-oxazol-2-yl]-benzyl}-acetamide;
2-(thien-3-yl)-N-{4-[4-([1,4]oxazepan-4-ylcarbonyl)-oxazol-2-yl]-benzyl}-acetamide;
2-(thien-3-yl)-N-{4-[4-(2-trifluoromethylpyrrolidin-1-ylcarbonyl)-oxazol-2-yl]-benzyl}-acetamide;
2-(thien-3-yl)-N-{4-[4-(4-trifluoromethylpiperidin-1-ylcarbonyl)-oxazol-2-yl]-benzyl}-acetamide;
2-(thien-3-yl)-N-{4-[4-(3-aza-bicyclo[3.1.0]hexan-3-ylcarbonyl)-oxazol-2-yl]-benzyl}-acetamide;
2-phenyl-N-{4-[4-(cis-3-hydroxy-4-hydroxymethylpiperidin-1-ylcarbonyl)-oxazol-2-yl]-benzyl}-acetamide;
2-phenyl-N-{4-[4-(trans-3-hydroxy-4-hydroxymethylpiperidin-1-ylcarbonyl)-oxazol-2-yl]-benzyl}-acetamide;
2-phenyl-N-{4-[4-(cis-4-hydroxy-3-hydroxymethylpiperidin-1-ylcarbonyl)-oxazol-2-yl]-benzyl}-acetamide;
2-(thien-3-yl)-N-{4-[4-(cis-3-hydroxy-4-hydroxymethylpiperidin-1-ylcarbonyl)-oxazol-2-yl]-benzyl}-acetamide;
2-(thien-3-yl)-N-{4-[4-(trans-3-hydroxy-4-hydroxymethylpiperidin-1-ylcarbonyl)-oxazol-2-yl]-benzyl}-acetamide;
2-(thien-3-yl)-N-{4-[4-(cis-4-hydroxy-3-hydroxymethylpiperidin-1-ylcarbonyl)-oxazol-2-yl]-benzyl}-acetamide;
2-phenyl-N-{4-[4-(8-oxa-3-aza-bicyclo [4.2.0]octan-3-ylcarbonyl)-oxazol-2-yl]-benzyl}-acetamide;
2-phenyl-N-{4-[4-(7-oxa-3-aza-bicyclo[4.2.0]octan-3-ylcarbonyl)-oxazol-2-yl]-benzyl}-acetamide;
2-phenyl-N-{4-[4-(4-acetylhomopiperazin-1-ylcarbonyl)-oxazol-2-yl]-benzyl}-amide;
2-(thien-3-yl)-N-{4-[4-(8-oxa-3-aza-bicyclo [4.2.0]octan-3-ylcarbonyl)-oxazol-2-yl]-yl}-acetamide;
2-(thien-3-yl)-N-{4-[4-(7-oxa-3-aza-bicyclo [4.2.0]octan-3-ylcarbonyl)-oxazol-2-yl]-yl}-acetamide;
2-(thien-3-yl)-N-{4-[4-(4-acetylhomopiperazin-1-ylcarbonyl)-oxazol-2-yl]-benzyl}-amide.

Representative compounds of Formulae I and II where R² is hydrogen, Y is —NHCO—, other groups are as specified below are:

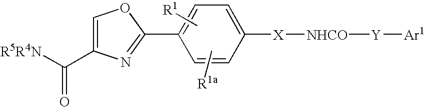

| Cmpd. | | | | | | |
|---|---|---|---|---|---|---|
| No. | —NR⁴R⁵ | R¹ | R^{1a} | X | Z | Ar¹ |
| 21839 | piperidin-1-yl | 5-fluoro | 3-fluoro | CH₂ | CH₂ | thien-3-yl |
| 21994 | piperidin-1-yl | H | 3-methyl | CH₂ | CH₂ | thien-3-yl |
| 21837 | piperidin-1-yl | 5-fluoro | 3-fluoro | CH₂ | CH₂ | phenyl |
| 21838 | piperidin-1-yl | 5-fluoro | 3-fluoro | CH₂ | CH₂ | 4-trifluoro- |
| | | | | | | methoxyphenyl |
| 21990 | piperidin-1-yl | H | 3-methyl | CH₂ | CH₂ | phenyl |
| 21991 | piperidin-1-yl | H | 3-methyl | CH₂ | CH₂ | 4-trifluoro- |
| | | | | | | methoxyphenyl |
| 21992 | piperidin-1-yl | H | 3-methyl | CH₂ | CH₂ | 4-methoxyphenyl |
| 21993 | piperidin-1-yl | H | 3-methyl | CH₂ | CH₂ | thien-2-yl |
| 22013 | piperidin-1-yl | H | 2-methoxy | CH₂ | CH₂ | phenyl |
| 22014 | piperidin-1-yl | H | 2-methoxy | CH₂ | CH₂ | thien-2-yl |
| 22328 | piperidin-l-yl | H | 3-methyl | CH₂ | CHF | phenyl |
| 22329 | piperidin-1-yl | H | 3-methyl | CH₂ | S—CHOH | phenyl |
| 22330 | piperidin-1-yl | H | 3-methyl | CH₂ | R—CHOH | phenyl |
| 22331 | piperidin-1-yl | H | 3-methyl | CH₂ | S—CHNH₂ | phenyl |
| 23128 | piperidin-1-yl | H | 2-hydroxy | CH₂ | CH₂ | phenyl |
| 23449 | piperidin-1-yl | H | 3-hydroxy | CH₂ | CH₂ | phenyl |
| 23542 | 4-hydroxy- | H | 2-iodo | CH₂ | CHF | phenyl |
| | piperidin-1-yl | | | | | |
| 23550 | 3,3-difluoro- | H | 2-nitro | CH₂ | CH₂ | phenyl |
| | piperidin-1-yl | | | | | |
| 23561 | 3,3-difluoro- | H | 2-iodo | CH₂ | CH₂ | phenyl |
| | piperidin-1-yl | | | | | |
| 23623 | 3,3-difluoro- | H | 2-(2-CH₃O₂C- | CH₂ | CH₂ | phenyl |
| | piperidin-1-yl | | ethylen-1-yl) | | | |
| 23624 | 3,3-difluoro- | H | 2-(2-CH₃O₂C-ethyl)- | CH₂ | CH₂ | phenyl |
| | piperidin-1-yl | | | | | |
| 23625 | 3,3-difluoro- | H | 2-(2-HO₂C-ethylen- | CH₂ | CH₂ | phenyl |
| | piperidin-1-yl | | 1-yl) | | | |
| 23649 | 3,3-difluoro- | H | 2-amino | CH₂ | CH₂ | phenyl |
| | piperidin-1-yl | | | | | |
| 23650 | 3,3-difluoro- | H | 2-acetylamino | CH₂ | CH₂ | phenyl |
| | piperidin-1-yl | | | | | |
| 23741 | 4-hydroxy- | H | 2-(2-HO₂C-ethyl)- | CH₂ | CH₂ | phenyl |
| | piperidin-1-yl | | | | | |
| 23776 | 3,3-difluoro- | H | 2-iodo | CH₂ | CF₂ | thien-2-yl |
| | piperidin-1-yl | | | | | |
| 23798 | 3,3-difluoro- | H | 2-(2-CH₃O₂C- | CH₂ | CF2 | thien-2-yl |
| | piperidin-1-yl | | ethylen-1-yl) | | | |
| 24100 | 3,3-difluoro- | H | 2-(2-HO₂C- | CH₂ | CH₂ | phenyl |
| | piperidin-1-yl | | ethyl)carbonylamino | | | |
| 24102 | 3,3-difluoro- | H | 2-(2-CH₃O₂C-ethyl)- | CH₂ | CF₂ | thien-2-yl |
| | piperidin-1-yl | | | | | |
| 24103 | 3,3-difluoro- | H | 2-(2-HO₂C-ethyl)- | CH₂ | CF₂ | thien-2-yl |
| | piperidin-1-yl | | | | | |
| 24233 | 3,3-difluoro- | H | 2-methoxycarbonyl | CH₂ | CF₂ | thien-2-yl |
| | piperidin-1-yl | | | | | |
| 24426 | 4-hydroxy- | 5-fluoro | 3-fluoro | CH₂ | CH₂ | thien-3-yl |
| | piperidin-1-yl | | | | | |
| 24452 | piperidin-1-yl | H | 3-methoxy | CH₂ | CH₂ | phenyl |
| 24460 | 3,3-difluoro- | H | 2-carboxy | CH₂ | CF₂ | thien-2-yl |
| | piperidin-1-yl | | | | | |
| 24497 | 3,3-difluoro- | H | 2-(piperazin-1-yl- | CH₂ | CH₂ | phenyl |
| | piperidin-1-yl | | carbonylethyl) | | | |
| 24506 | 4-hydroxy- | H | 2-(morpholin-4-yl- | CH₂ | CH₂ | phenyl |
| | piperidin-1-yl | | carbonylethyl) | | | |
| 24509 | piperidin-1-yl | H | 3-methoxycarbonyl- | CH₂ | CH₂ | phenyl |
| | | | methyloxy | | | |
| 24768 | 4-hydroxy- | H | 2-methoxycarbonyl | CH₂ | CH₂ | phenyl |
| | piperidin-1-yl | | | | | |
| 25009 | 3- | 5-fluoro | 3-fluoro | CH₂ | CH₂ | thien-3-yl |
| | hydroxypiperidin- | | | | | |
| | 1-yl | | | | | |
| 25011 | 4- | 5-fluoro | 3-fluoro | CH₂ | CH₂ | thien-3-yl |
| | oxopiperidin- | | | | | |
| | 1-yl | | | | | |
| 25010 | trans-3,4- | 5-fluoro | 3-fluoro | CH₂ | CH₂ | thien-3-yl |
| | dihydroxypiperidin- | | | | | |
| | 1-yl | | | | | |
| 24984 | pyrrolidin-1- | 5-fluoro | 3-fluoro | CH₂ | CH₂ | thien-3-yl |
| | yl | | | | | |

and are named as
2-(thien-3-yl)-N-{4-[4-(piperidin-1-ylcarbonyl)-oxazol-2-yl]-(3,5-difluorophenylmethyl)}-acetamide;
2-(thien-3-yl)-N-{4-[4-(piperidin-1-ylcarbonyl)-oxazol-2-yl]-(3-methylphenylmethyl)}-acetamide;
2-phenyl-N-{4-[4-(piperidin-1-ylcarbonyl)-oxazol-2-yl]-(3,5-difluorophenylmethyl)}-acetamide;
2-(4-trifluoromethoxyphenyl)-N-{4-[4-(piperidin-1-ylcarbonyl)-oxazol-2-yl]-(3,5-difluorophenylmethyl)}-acetamide;
2-phenyl-N-{4-[4-(piperidin-1-ylcarbonyl)-oxazol-2-yl]-(3-methylphenylmethyl)}-acetamide;
2-(4-trifluoromethoxyphenyl)-N-{4-[4-(piperidin-1-ylcarbonyl)-oxazol-2-yl]-(3-methylphenylmethyl)}-acetamide;
2-(4-methoxyphenyl)-N-{4-[4-(piperidin-1-ylcarbonyl)-oxazol-2-yl]-(3-methylphenylmethyl)}-acetamide;
2-(thien-2-yl)-N-{4-[4-(piperidin-1-ylcarbonyl)-oxazol-2-yl]-(3-methylphenylmethyl)}-acetamide;
2-phenyl-N-{4-[4-(piperidin-1-ylcarbonyl)-oxazol-2-yl]-(2-methoxyphenylmethyl)}-acetamide;
2-(thien-2-yl)-N-{4-[4-(piperidin-1-ylcarbonyl)-oxazol-2-yl]-(2-methoxyphenylmethyl)}-acetamide;
2-fluoro-2-phenyl-N-{4-[4-(piperidin-1-ylcarbonyl)-oxazol-2-yl]-(3-methylphenylmethyl)}-acetamide;
2S-hydroxy-2-phenyl-N-{4-[4-(piperidin-1-ylcarbonyl)-oxazol-2-yl]-(3-methylphenylmethyl)}-acetamide;
2R-hydroxy-2-phenyl-N-{4-[4-(piperidin-1-ylcarbonyl)-oxazol-2-yl]-(3-methylphenylmethyl)}-acetamide;
2S-amino-2-phenyl-N-{4-[4-(piperidin-1-ylcarbonyl)-oxazol-2-yl]-(3-methylphenylmethyl)}-acetamide
2-phenyl-N-{4-[4-(piperidin-1-ylcarbonyl)-oxazol-2-yl]-(2-hydroxyphenylmethyl)}-acetamide;
2-phenyl-N-{4-[4-(piperidin-1-ylcarbonyl)-oxazol-2-yl]-(3-hydroxyphenylmethyl)}-acetamide;
2-fluoro-2-phenyl-N-{4-[4-(4-hydroxypiperidin-1-ylcarbonyl)-oxazol-2-yl]-2-iodophenylmethyl}-acetamide;
2-phenyl-N-{4-[4-(3,3-difluoropiperidin-1-ylcarbonyl)-oxazol-2-yl]-2-nitrophenylmethyl}-acetamide;
2-phenyl-N-{4-[4-(3,3-difluoropiperidin-1-ylcarbonyl)-oxazol-2-yl]-2-iodophenylmethyl}-acetamide;
2-phenyl-N-{4-[4-(3,3-difluoropiperidin-1-ylcarbonyl)-oxazol-2-yl]-2-(methoxycarbonylethylen-1-yl)phenylmethyl}-acetamide;
2-phenyl-N-{4-[4-(3,3-difluoropiperidin-1-ylcarbonyl)-oxazol-2-yl]-2-(methoxycarbonylethyl)phenylmethyl}-acetamide;
2-phenyl-N-{4-[4-(3,3-difluoropiperidin-1-ylcarbonyl)-oxazol-2-yl]-2-(carboxyethylen-1-yl)phenylmethyl}-acetamide;
2-phenyl-N-{4-[4-(3,3-difluoropiperidin-1-ylcarbonyl)-oxazol-2-yl]-2-aminophenylmethyl}-acetamide;
2-phenyl-N-{4-[4-(3,3-difluoropiperidin-1-ylcarbonyl)-oxazol-2-yl]-2-acetylaminophenylmethyl}-acetamide;
2-phenyl-N-{4-[4-(4-hydroxpiperidin-1-ylcarbonyl)-oxazol-2-yl]-2-carboxyethylphenylmethyl}-acetamide;
2,2-difluoro-2-(thien-2-yl)-N-{4-[4-(3,3-difluoropiperidin-1-ylcarbonyl)-oxazol-2-yl]-2-iodophenylmethyl}-acetamide;
2,2-difluoro-2-(thien-2-yl)-N-{4-[4-(3,3-difluoropiperidin-1-ylcarbonyl)-oxazol-2-yl]-2-(methoxycarbonylethylen-1-yl)phenylmethyl}-acetamide;
2-phenyl-N-{4-[4-(3,3-difluoropiperidin-1-ylcarbonyl)-oxazol-2-yl]-2-(2-carboxyethylcarbonylamino)phenylmethyl}-acetamide;
2,2-difluoro-2-(thien-2-yl)-N-{4-[4-(3,3-difluoropiperidin-1-ylcarbonyl)-oxazol-2-yl]-2-methoxycarbonylethylphenylmethyl}-acetamide;
2,2-difluoro-2-(thien-2-yl)-N-{4-[4-(3,3-difluoropiperidin-1-ylcarbonyl)-oxazol-2-yl]-2-carboxyethylphenylmethyl}-acetamide;
2,2-difluoro-2-(thien-2-yl)-N-{4-[4-(3,3-difluoropiperidin-1-ylcarbonyl)-oxazol-2-yl]-2-methoxycarbonylphenylmethyl}-acetamide;
2-(thien-3-yl)-N-{4-[4-(4-hydroxypiperidin-1-ylcarbonyl)-oxazol-2-yl]-3,5-difluorophenylmethyl}-acetamide;
2-phenyl-N-{4-[4-(piperidin-1-ylcarbonyl)-oxazol-2-yl]-3-methoxyphenylmethyl}-acetamide;
2,2-difluoro-2-(thien-2-yl)-N-{4-[4-(3,3-difluoropiperidin-1-ylcarbonyl)-oxazol-2-yl]-2-carboxyphenylmethyl}-acetamide;
2-phenyl-N-{4-[4-(3,3-difluoropiperidin-1-ylcarbonyl)-oxazol-2-yl]-2-(piperazin-1-ylcarbonylethyl)phenylmethyl}-acetamide;
2-phenyl-N-{4-[4-(4-hydroxypiperidin-1-ylcarbonyl)-oxazol-2-yl]-2-(morpholin-4-ylcarbonylethyl)phenylmethyl}-acetamide;
2-phenyl-N-{4-[4-(piperidin-1-ylcarbonyl)-oxazol-2-yl]-3-methoxycarbonylmethyloxyphenylmethyl}-acetamide;
2-phenyl-N-{4-[4-(4-hydroxypiperidin-1-ylcarbonyl)-oxazol-2-yl]-2-methoxycarbonylphenylmethyl}-acetamide;
2-(thien-3-yl)-N-{4-[4-(3-hydroxypiperidin-1-ylcarbonyl)-oxazol-2-yl]-3,5-difluorophenylmethyl}-acetamide;
2-(thien-3-yl)-N-{4-[4-(4-oxopiperidin-1-ylcarbonyl)-oxazol-2-yl]-3,5-difluorophenylmethyl}-acetamide;
2-(thien-3-yl)-N-{4-[4-(trans-3,4-dihydroxypiperidin-1-ylcarbonyl)-oxazol-2-yl]-3,5-difluorophenylmethyl}-acetamide;
2-(thien-3-yl)-N-{4-[4-(pyrrolidin-1-ylcarbonyl)-oxazol-2-yl]-3,5-difluorobenzyl}-acetamide.

Representative compounds of Formulae I and II where R¹, R^{1a} and R² are hydrogen and other groups are as specified below are:

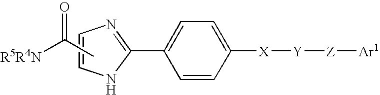

| Cmpd. | | | | | |
|---|---|---|---|---|---|
| No. | —NR⁴R⁵ | X | Y | Z | Ar¹ |
| 22452 | 3,3-difluoro- | CH₂ | NHCO | CH₂ | phenyl |
| | piperidin-1-yl | | | | |
| 22482 | 2,5-dimethyl- | CH₂ | NHCO | CH₂ | phenyl |
| | pyrrolidin-1-yl | | | | |

and are named as
2-phenyl-N-{4-[4-(3,3-difluoropiperidin-1-yl-carbonyl)-1H-imidazol-2-yl]-benzyl}-acetamide; and
2-phenyl-N-{4-[4-(2,5-dimethylpyrrolidin-1-yl-carbonyl)-1H-imidazol-2-yl]-benzyl}-acetamide.

Representative compounds of Formulae I and II where R^{1 and R}^{1a} are hydrogen and other groups are as specified below are:

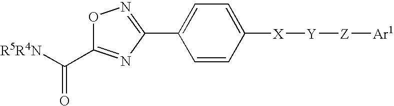

| Cmpd. | | | | | |
|---|---|---|---|---|---|
| No. | R³ | X | Y | Z | Ar¹ |
| 22489 | piperidin-1-yl | CH₂ | NHCO | CH₂ | phenyl |

and is named as 2-phenyl-N-{4-[5-(piperidin-1-yl-carbonyl)-[1,2,4]oxadiazol-3-yl]-benzyl}-acetamide.

Representative compounds of Formulae I and II where R¹, R^{1a}, and R² are hydrogen and other groups are as specified below are:

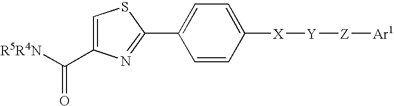

| Cmpd. | | | | | |
|---|---|---|---|---|---|
| No. | R³ | X | Y | Z | Ar¹ |
| 20945 | piperidin-1-yl | CH₂ | NHCO | CH₂ | phenyl |
| 20946 | piperidin-1-yl | CH₂ | NHCO | CH₂ | thien-2-yl |
| 20947 | piperidin-1-yl | CH₂ | NHSO₂ | CH₂ | phenyl |
| 20948 | piperidin-1-yl | CH₂ | NHCO | CH₂CH₂ | phenyl |

and are named as:
2-phenyl-N-{4-[4-(piperidin-1-ylcarbonyl)-thiazol-2-yl]-benzyl}-acetamide;
2-(thien-2-yl)-N-{4-[4-(piperidin-1-ylcarbonyl)-thiazol-2-yl]-benzyl}-acetamide;
1-phenyl-N-{4-[4-(piperidin-1-ylcarbonyl)-thiazol-2-yl]-benzyl}-methanesulfonamide;
3-phenyl-N-{4-[4-(piperidin-1-ylcarbonyl)-thiazol-2-yl]-benzyl}-propionamide.

Representative compounds of Formulae I and II where R¹, R^{1a}, R², and R²′ are hydrogen and other groups are as specified below are:

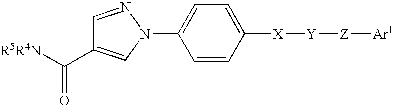

| Cmpd. | | | | | |
|---|---|---|---|---|---|
| No. | R³ | X | Y | Z | Ar¹ |
| 22618 | 3,3-difluoro- | CH₂ | NHCO | CH₂ | thien-3-yl |
| | piperidin-1-yl- | | | | |
| 22620 | piperidin-1-yl- | CH₂ | NHCO | CH₂ | thien-3-yl |
| 22612 | piperidin-1-yl- | CH₂ | NHCO | CH₂ | phenyl |
| 22613 | 3-hydroxy- | CH₂ | NHCO | CH₂ | phenyl |
| | piperidin-1-yl | | | | |
| 22614 | 4-hydroxy- | CH₂ | NHCO | CH₂ | phenyl |
| | piperidin-1-yl- | | | | |
| 22615 | 3,3-difluoro- | CH₂ | NHCO | CH₂ | phenyl |
| | piperidin-1-yl- | | | | |
| 22616 | 3-hydroxy- | CH₂ | NHCO | CH₂ | thien-3-yl |
| | piperidin-1-yl- | | | | |
| 22617 | 4-hydroxy- | CH₂ | NHCO | CH₂ | thien-3-yl |
| | piperidin-1-yl | | | | |

are named as
2-(thien-3-yl)-N-{4-[4-(3,3-difluoropiperidin-1-ylcarbonyl)-pyrazol-1-yl]-benzyl}-acetamide;
2-(thien-3-yl)-N-{4-[4-(piperidin-1-ylcarbonyl)-pyrazol-1-yl]-benzyl}-acetamide;
2-phenyl-N-{4-[4-(piperidin-1-ylcarbonyl)-pyrazol-1-yl]-benzyl}-acetamide;
2-phenyl-N-{4-[4-(3-hydroxypiperidin-1-ylcarbonyl)-pyrazol-1-yl]-benzyl}-acetamide;
2-phenyl-N-{4-[4-(4-hydroxypiperidin-1-ylcarbonyl)-pyrazol-1-yl]-benzyl}-acetamide;
2-phenyl-N-{4-[4-(3,3-difluoropiperidin-1-ylcarbonyl)-pyrazol-1-yl]-benzyl}-acetamide;
2-(thien-3-yl)-N-{4-[4-(3-hydroxypiperidin-1-ylcarbonyl)-pyrazol-1-yl]-benzyl}-acetamide;
2-(thien-3-yl)-N-{4-[4-(4-hydroxypiperidin-1-ylcarbonyl)-pyrazol-1-yl]-benzyl}-15 acetamide.

### General Synthesis

Compounds of this invention can be made by the synthetic procedures described below.

The starting materials and reagents used in preparing these compounds are either available from commercial suppliers such as Aldrich Chemical Co., (Milwaukee, Wis.), or Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4^{th} Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula I or II can be prepared by the procedure illustrated and described in Schemes A-E below:

A compound of Formula I or II where R is alkyl, Y is —NH—, —O—, or —S—, and Het, R¹, R^{1a}, R², and R³ are as defined in the Summary of the Invention can be prepared as illustrated and described below.

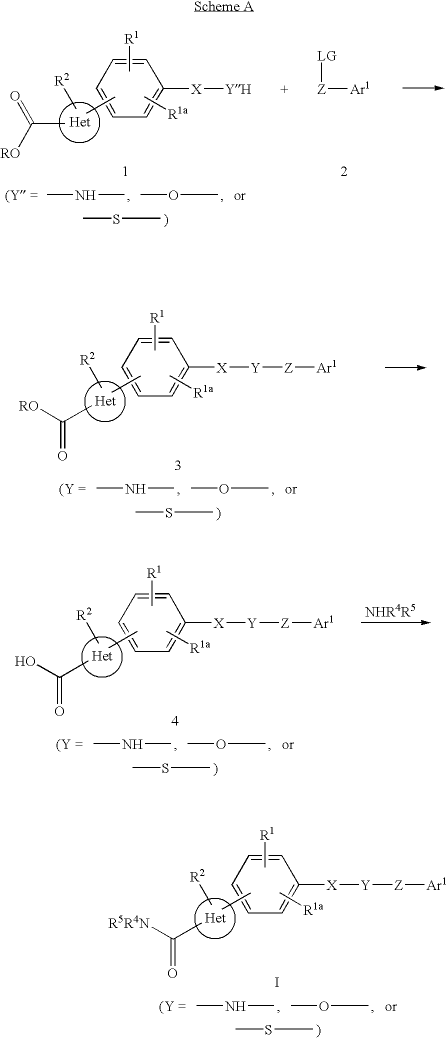

Reaction of a compound of formula 1 with an alkylating compound of formula 2 where LG is a suitable leaving group such as halo, mesylate, tosylate, or triflate provides a compound of formula 3. The reaction is carried out in the presence of a base e.g., sodium carbonate, potassium carbonate, sodium hydride and the like. Suitable solvents for the reaction are THF, dioxane, N,N-dimethylformamide and the like.

Compounds of formula 1 can be prepared by methods well known in the art. Detailed description of syntheses of compound of formula 1 where Het is oxazol-2-yl, thiazol-2-yl, pyrazol-1-yl, imidazol-2-yl or [1,2,3]oxadiazol-3-yl are given in working examples below. Compounds of formula 2 are commercially available or they can be prepared from readily available starting materials by methods well known in the art. For example, benzyl chloride, 2-, 3-, 4-fluorobenzyl bromide, and 4-(chloromethyl)-3,5-dimethylisoxazole are commercially available. Compound 2 where LG is mesylate, tosylate, or triflate can be prepared from corresponding alcohols by reaction with mesyl chloride, tosyl chloride, or trifloromethanesulfonyl chloride respectively, in the presence of a base. Alcohols such as benzyl alcohol, thienyl ethanol are commercially available.

Compounds of formula 3 where Y is —NH— can also be prepared by reacting 1 with an aldehyde of formula Ar¹—Z—CHO under reductive amination reaction conditions.

Hydrolysis of the ester group in 3 provides a compound of formula 4. The hydrolysis is carried out in the presence of an aqueous base such as aqueous sodium hydroxide, lithium hydroxide, and the like in a suitable organic solvent such as methanol, ethanol, THF, and the like.

Compound 4 is then converted to a compound of Formula I or II by first converting 4 to a reactive acid derivative followed by treatment an amine of formula NHR⁴R⁵. Specifically, 4 can be first converted to an acid halide derivative such as acid chloride, and the like with a chlorinating agent such as thionyl chloride, oxalyl chloride, and the like. Suitable solvents are halogenated organic solvents such as methylene chloride, and the like. The resulting acid halide is then reacted with an amine of formula NHR⁴R⁵. The amination reaction is carried out in the presence of a suitable base such as triethylamine, pyridine, and the like and in a suitable organic solvent such as THF, dioxane, N,N-dimethylformamide and the like.

Alternatively, a compound of Formula I or II can be prepared by reacting 4 with the amine in the presence of a coupling agent such as benzotriazole-1-yloxytrispyrrolidino-phosphonium hexafluorophosphate (PyBOP®), bromo-tris-pyrrolidino-phosphonium hexafluorophosphate (PyBrop®), O-benzotriazol-1-yl-N,N,N′,N′-tetramethyl-uronium hexafluorophosphate (HBTU), O-(7-azabenzotriazol-1-yl)-N,N,N′,N′-tetramethyluronium hexafluorophosphate (HATU), or 1-hydroxybenzotriazole (HOBT) in the presence of 1,3-dicyclohexylcarbodiimide (DCC) or 1-(3-dimethylaminopropyl)-3-ethylcarbodiimide hydrochloride (EDC), or a base such as N,N-diisopropylethylamine, triethylamine, or N-methylmorpholine. Suitable solvents are dichloromethane, dioxane, dichloroethane, dimethylformamide, tetrahydrofuran, or acetonitrile.

Amines of formula NHR⁴R⁵ such as piperidine, pyrrolidine, piperazine, morpholine, tetrahydropyridine, homopiperazine, hydroxypiperidine, and the like are commercially available or can be prepared readily according to literature methods.

A compound of Formula I or II where R is alkyl, Y is —NHCO— and Het, R¹, R^{1a}, R² and R³ are as defined in the Summary of the Invention can be prepared as illustrated and described in Scheme B below.

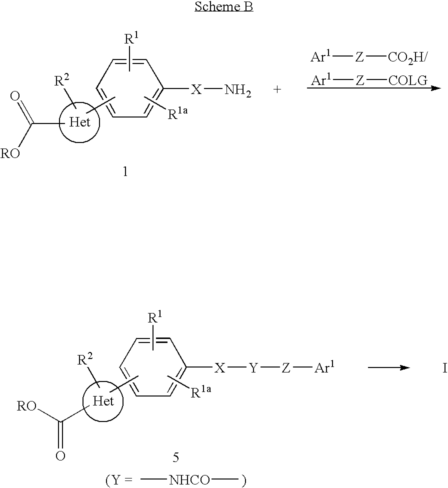

A compound of Formula I or II where Y is —NHCO— can be prepared by reacting 1 with an acid of formula Ar¹—Z—COOH or an acylating reagent of the formula Ar¹—Z—COLG where LG is a leaving group under acylating conditions, such as a halo such as chloro, bromo, and the like to provide a compound of formula 5. If Ar¹—Z—COOH is utilized in the reaction then it is carried out under coupling reaction conditions described in Scheme A above. If an acyl halide is used as the acylating agent the reaction is carried out in the presence of a non-nucleophilic organic base such as triethylamine, pyridine, and the like. Examples of solvents of the reaction include dichloromethane, THF, dioxane, DMF, and the like. Acylating agents of the formula Ar¹—Z—COLG can be prepared by reacting the corresponding acid of formula Ar¹—Z—CO₂H with a chlorinating or brominating agent under the conditions described above. Compound 5 is then converted to a compound of Formula I or II as described in Scheme A above.

Compounds of Formula I or II where Y is —NHSO₂— can be prepared as described in Scheme B above by substituting the acyl halide with a sulfonyl halide of the formula Ar¹—Z—SO₂LG utilizing the reaction conditions described above. Sulfonyl halides are commercially available or may be prepared by methods well known in the art.

A compound of Formula I or II where R is alkyl, Y is —NHCONH— and Het, R¹, R^{1a}, R² and R³ are as defined in the Summary of the Invention can be prepared as illustrated and described in Scheme C below.

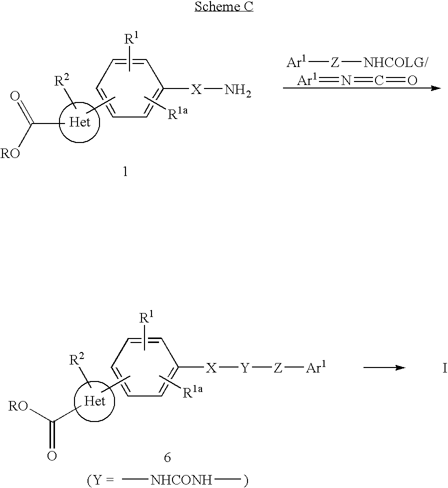

A compound of Formula I or II where Y is —NHCONH— can be prepared by converting a compound of formula 1 to a compound of formula 6 by either:
i) reacting 1 with a carbamoyl halide of formula Ar¹—Z—NHCOLG. The reaction is carried out in the presence of a non-nucleophilic organic base. Suitable solvents for the reaction are dichloromethane, 1,2-dichloroethane, THF, and the like; or
ii) reacting 1 with an isocyanate in an organic solvent such as benzene, THF, dimethylformamide, and the like.

Compound 6 is then converted to a compound of Formula I or II as described in Scheme A above.

The procedures described in Scheme C above can also be used to synthesize compounds of Formula I or II where Y is —NHCSNH— by substituting carbamoyl halide with sulfamoyl halide and isocyanate with isothiocyanate respectively.

A compound of Formula I or II where R is alkyl, Y is —OCONH— and Het, R¹, R^{1a}, R² and R³ are as defined in the Summary of the Invention can be prepared as illustrated and described in Scheme D below.

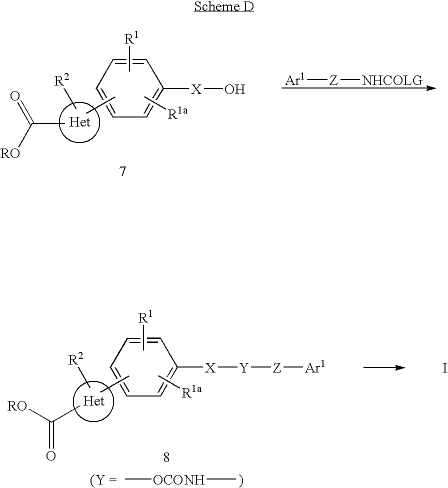

A compound of Formula I or II where Y is —OCONH— can be prepared by converting a compound of formula 1 to a compound of formula 7 under the reaction conditions described in U.S. Pat. No. 6,136,844, followed by reaction with a carbamoyl halide under the reaction conditions described in Scheme C above.

Alternatively, a compound of Formula I or II can be prepared as illustrated and described in Scheme E below.

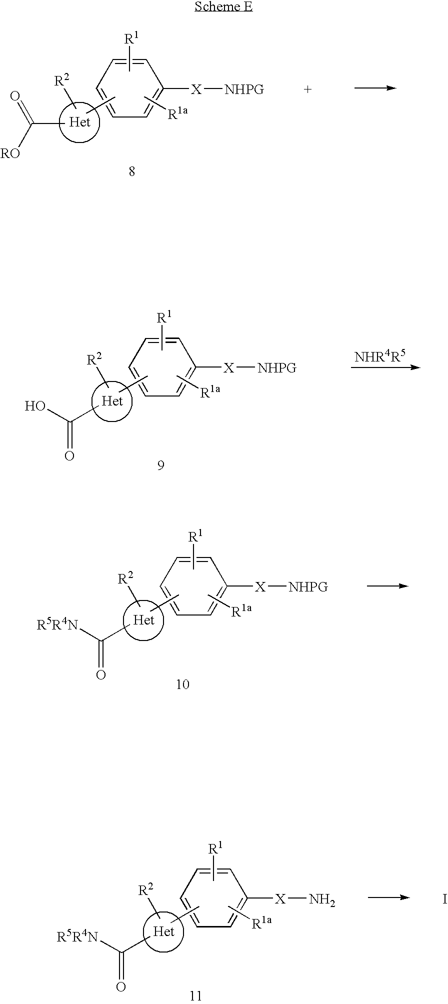

A compound of Formula I or II can alternatively be prepared by first converting a compound of formula 8 (where PG is a suitable amino protecting group such as tert-butoxycarbonyl, benzyl, CBz, and the like and other groups are as defined in the Summary of the Invention) to a compound of formula 10 under the reaction conditions described in Scheme A above, followed by removal of the amino protecting group to provide a compound of formula 11. The reaction conditions for removal of amino protecting group depend on the nature of the protecting group. For example, if it is tert-butoxycarbonyl it is removed under acidic hydrolysis reaction conditions. If it is benzyl it is removed under hydrogenation reaction conditions. A comprehensive list of suitable protective groups can be found in T. W. Greene, Protective Groups in Organic Synthesis, John Wiley & Sons, Inc. 1981, the disclosure of which is incorporated herein by reference in its entirety.

Compound 11 is then converted to a compound of Formula I or II as described in Schemes A-D above.

Compound of Formula I can be converted to other compounds of Formula I by methods well known in the art. For example, a compound of Formula I where R¹ is nitro can be converted to a corresponding compound of Formula I where R¹ is amino by reduction of the amino group under catalytic hydrogenation reaction conditions. A compound of Formula I where R¹ is amino can be converted to a corresponding compound of Formula I where R¹ is dialkylamino by reacting it with an alkylating agent such as alkyl halide in the presence of a base. A compound of Formula I where R¹ is acylamino can be prepared by reacting a corresponding compound of Formula I where R¹ is amino with an acylating agent such as acyl halide in the presence of a base.

### Utility

The compounds of this invention are activators of caspases and inducers of apoptosis and are therefore useful in the treatment of a disease in which caspase cascade mediated physiological responses are implicated. In particular the compounds of this invention are useful in the treatment of proliferative diseases such as cancer which includes, but are not limited to, Hodgkin's disease, non-Hodgkin's lymphomas, acute and chronic lymphocytic leukemias, multiple myeloma, neuroblastoma, breast carcinomas, ovarian carcinomas, lung carcinomas, Wilms' tumor, cervical carcinomas, testicular carcinomas, soft tissue sarcomas, chronic lymphocytic leukemia, primary macroglobulinemia, bladder carcinomas, chronic granulocytic leukemia, primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, neuroblastoma, rhabdomyo sarcoma, Kaposi's sarcoma, genitourinary carcinomas, thyroid carcinomas, esophageal carcinomas, malignant hypercalcemia, cervical hyperplasia, renal cell carcinomas, endometrial carcinomas, polycythemia vera, essential thrombocytosis, adrenal cortex carcinomas, skin cancer, and prostatic carcinomas.

A wide range of immune mechanisms operate rapidly following exposure to an infectious agent. Depending on the type of infection, rapid clonal expansion of the T and B lymphocytes occurs to combat the infection. The elimination of the effector cells following an infection is one of the major mechanisms maintaining immune homeostasis. This deletion of reactive cell has been shown to be regulated by a phenomenon known as apoptosis. Autoimmune diseases have been lately identified as a consequence of deregulated cell death. In certain autoimmune diseases, the immune system directs its powerful cytotoxic effector mechanisms against specialized cells such as oligodendrocytes in multiple sclerosis, the beta cells of the pancreas in diabetes mellitus, and thyrocytes in Hashimoto's thyroiditis (Ohsako. S. & Elkon, K. B., Cell Death Differ. 6:13-21 (1999)). Mutations of the gene encoding the lymphocyte apoptosis receptor Fas/APO-1/CD95 are reported to be associated with defective lymphocyte apoptosis and autoimmune lymphoproliferative syndrome (ALPS), which is characterized by chronic, histologically benign splenomegaly and generalized lymphadenopathy, hypergammaglobulinemia, and autoantibody formation (Infante, A. J., et al., J Pediatr. 133:629-633 (1998) and Vaishnaw, A. K., et al., J. Clin. Invest. 103:355-3)₆₃ (1999)).

Overexpression of Bcl-2, which is a member of the bcl-2 gene family of programmed cell death regulators with anti-apoptotic activity in developing B cells of transgenic mice, in the presence of T cell dependent co-stimulatory signals, results in the generation of a modified B cell repertoire and in the production of pathogenic autoantibodies (Lopez-Hoyos, M., et al., Int. J. Mol. Med. 1:475-483 (1998)).

Accordingly, many types of autoimmune disease may be caused by defects of the apoptotic process, and one treatment strategy would be to turn on apoptosis in the lymphocytes that are causing autoimmune disease (O'Reilly, L. A. & Strasser, A., Inflamm. Res. 48:5-21 (1999)).

Fas-Fas ligand (FasL) interaction is known to be required for the maintenance of immune homeostasis. Experimental autoimmune thyroiditis (EAT), characterized by autoreactive T and B cell responses and a marked lymphocytic infiltration of the thyroid, is a good model to study the therapeutic effects of FasL. Batteux, F., et. al., (J. Immunol. 162:603-608 (1999)) reported that by direct injection of DNA expression vectors encoding FasL into the inflamed thyroid, the development of lymphocytic infiltration of the thyroid was inhibited and induction of infiltrating T cells death was observed. These results show that FasL expression on thyrocytes may have a curative effect on ongoing EAT by inducing death of pathogenic autoreactive infiltrating T lymphocytes.

Bisindolylmaleimide VIII is known to potentiate Fas-mediated apoptosis in human astrocytoma 1321NI cells and in Molt-4T cells, and both of which were resistant to apoptosis induced by anti-Fas antibody in the absence of bisindolylmaleimide VIII. Potentiation of Fas-mediated apoptosis by bisindolylmaleimide VIII was reported to be selective for activated, rather than non-activated, T cells, and was Fas-dependent. Zhou T., et al., (Nat. Med 5:42-49 (1999)) reported that administration of bisindolylmaleimide VIII to rats during autoantigen stimulation prevented the development of symptoms of T cell-mediated autoimmune diseases in two models, the Lewis rat model of experimental allergic encephalitis and the Lewis adjuvant arthritis model. Therefore the application of a Fas-dependent apoptosis enhancer such as bisindolylmaleimide VIII may be therapeutically useful for the more effective elimination of detrimental cells and inhibition of T cell-mediated autoimmune diseases. Therefore the compounds of this invention should be an effective in the treatment of autoimmune diseases.

Psoriasis is a chronic skin disease that is characterized by scaly red patches. Psoralen plus ultraviolet A (PUVA) is a widely used and effective treatment for psoriasis vulgaris and Coven, et al., Photodermatol. Photoimmunol. Photomed 15:22-27 (1999), reported that lymphocytes treated with psoralen 8-MOP or TMP plus UVA displayed DNA degradation patterns typical of apoptotic cell death. Ozawa, et al., J. Exp. Med 189:711-718 (1999) reported that induction of T cell apoptosis could be the main mechanism by which 312-nm UVB resolves psoriasis skin lesions. Low doses of methotrexate may be used to treat psoriasis to restore a clinically normal skin. Heenen, et al., Arch. Dermatol. Res. 290:240-245 (1998), reported that low doses of methotrexate may induce apoptosis and this mode of action could explain the reduction in epidermal hyperplasia during treatment of psoriasis with methotrexate. Therefore the compounds of this invention which function as a caspase cascade activator and inducer of apoptosis, should be effective in the treatment of psoriasis.

Synovial cell hyperplasia is a characteristic of patients with rheumatoid arthritis (RA). Excessive proliferation of RA synovial cells as well as defects in synovial cell death might be responsible for the synovial cell hyperplasia. Wakisaka, et al., Clin. Exp. Immunol. 114:119-128 (1998), found that although RA synovial cells could die via apoptosis through Fas/FasL pathway, apoptosis of synovial cells was inhibited by proinflammatory cytokines present within the synovium, and suggested that inhibition of apoptosis by the proinflammatory cytokines may contribute to the outgrowth of synovial cells, and lead to pannus formation and the destruction of joints in patients with RA. Therefore the compounds of this invention which function as a caspase cascade activator and inducer of apoptosis should also be effective in the treatment of rheumatoid arthritis.

An accumulation of convincing evidence suggests that apoptosis plays a major role in promoting resolution of the acute inflammatory response. Neutrophils are constitutively programmed to undergo apoptosis, thus limiting their pro-inflammatory potential and leading to rapid, specific, and non-phlogistic recognition by macrophages and semi-professional phagocytes (Savill, J., J. Leukoc. Biol. 61:375-380 (1997)). Boirivant, et al., Gastroenterology 116:557-565 (1999), reported that lamina propria T cells isolated from areas of inflammation in Crohn's disease, ulcerative colitis, and other inflammatory states manifest decreased CD2 pathway-induced apoptosis, and that studies of cells from inflamed Crohn's disease tissue indicate that this defect is accompanied by elevated Bcl-2 levels. Therefore the compounds of this invention which function as a caspase cascade activator and inducer of apoptosis should also be effective in the treatment of inflammation and inflammatory bowel disease.

### Administration and Pharmaceutical Compositions

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors.

Therapeutically effective amounts of compounds of Formula I or II may range from approximately 0.1-50 mg per kilogram body weight of the recipient per day; preferably about 0.5-20 mg/kg/day. Thus, for administration to a 70 kg person, the dosage range would most preferably be about 35 mg to 1.4 g per day. If a known chemotherapeutic agent is also administered, it is administered in an amount which is effective to achieve its intended purpose. The amounts of such known cancer chemotherapeutic agents effective for cancer are well known to those of skill in the art.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral or parenteral using a convenient daily dosage regimen, which can be adjusted according to the degree of affliction. Oral compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of Formula I or II in combination with at least one pharmaceutically acceptable excipient. Acceptable excipients are non-toxic, aid administration, and do not adversely affect the therapeutic benefit of the compound of Formula I or II. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18^{th} ed., 1990).

The amount of the compound in a formulation can vary within the full range employed by those skilled in the art. Typically, the formulation will contain, on a weight percent (wt %) basis, from about 0.01-99.99 wt % of a compound of Formula I or II based on the total formulation, with the balance being one or more suitable pharmaceutical excipients. Preferably, the compound is present at a level of about 1-80 wt %. Representative pharmaceutical formulations containing a compound of Formula I or II are described below.

As stated previously, the compounds of this invention can be administered in combination with known anti-cancer agents. Such known anti-cancer agents include the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors. The compound of the present invention compounds are particularly useful when adminsitered in combination with radiation therapy. Preferred angiogenesis inhibitors are selected from the group consisting of a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4 and analogues, squalamine, 6-O-chloroacetyl-carbonyl-fumagillol, thalidomide, angiostatin, troponin-1, and an antibody to VEGF.

Preferred estrogen receptor modulators are tamoxifen and raloxifene.

“Estrogen receptor modulators” refers to compounds that interfere or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H—benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylomithine, ILX23-7553, trans-N-(4′-hydroxyphenyl) retinamide, and N-4-carboxyphenyl retinamide.

“Cytotoxic agents” refer to compounds which cause cell death primarily by interfering directly with the cell's functioning or inhibit or interfere with cell mitosis, including alkylating agents, tumor necrosis factors, intercalators, microtubulin inhibitors, and topoisomerase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, diarizidinylspermine, GPX100, arsenic trioxide, (trans, trans, trans)-bis-mu-(hexane-1,6-diamine)-mu-[diamine-platinum(II)]bis[diamine(chloro)platinum(II)] tetrachloride, zorubicin, 1-(11-dodecylamino-10-hydroxyundecyl)-3,7-dimethylxanthine, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarmiinomycin, annamycin, galarubicin, elinafide, MEN10755, and 4-demethoxy-3-deamino-3-aziridinyl-4-methylsulphonyl-daunorubicin (see WO 00/50032).

Examples of microtubulin inhibitors include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, and BMS 188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)-ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′: 6,7)colchic(2,3-d)-1,3-dioxol-6-one, 2,3-(methylenedioxy)-5-methyl-7-hydroxy-8-methoxybenzo[c]-phenanthridinium, 6,9-bis [(2-aminoethyl)-amino]benzo[g]isoguinoline-5, 10-dione, 5-(3-aminopropylamino)-7, 10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]-adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetra cyclo(7.4.1.0.0)-tetradeca-2,4,6-trien-9-yl acetic acid ester, swainsonine, lometrexol, dexrazoxane, methioninase, 2′-cyano-2′-deoxy-N-4-palmitoyl-1-B-D-arabino furanosyl cytosine, and 3-aminopyridine-2-carboxaldehyde thiosemicarbazone. “Antiproliferative agents” also includes monoclonal antibodies to growth factors, other than those listed under “angiogenesis inhibitors”, such as trastuzumab, and tumor suppressor genes, such as p53, which can be delivered via recombinant virus-mediated gene transfer (see U.S. Pat. No. 6,069,134, for example).

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein. It has been reported that (Int. J. Cancer, 20;97(6):746-50, 2002) combination therapy with lovastatin, a HMG-CoA reductase inhibitor, and butyrate, an inducer of apoptosis in the Lewis lung carcinoma model in mice showed potentiating antitumor effects

Examples of HMG-CoA reductase inhibitors that may be used include but are not limited to lovastatin (MEVACOR®; see U.S. Pat. Nos. 4,231,938; 4,294,926; 4,319,039), simvastatin (ZOCOR®; see U.S. Pat. Nos. 4,444,784; 4,820,850; 4,916,239), pravastatin (PRAVACHOL®; see U.S. Pat. Nos. 4,346,227; 4,537,859; 4,410,629; 5,030,447 and 5,180,589), fluvastatin (LESCOL®; see U.S. Pat. Nos. 5,354,772; 4,911,165; 4,929,437; 5,189,164; 5,118,853; 5,290,946; 5,356,896), atorvastatin (LIPITOR®; see U.S. Pat. Nos. 5,273,995; 4,681,893; 5,489,691; 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (Feb. 5, 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and colchicin the use of such salts, esters, open-acid and lactone forms is included within the scope of this invention.

In HMG-CoA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin.

Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, piperazine, 1-p-chlorobenzyl-2-pyrrolidine-1′-yl-methylbenzimidazole, diethylamine, and tris(hydroxymethyl) aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). Examples of prenyl-protein transferase inhibiting compounds include (±)-6-[amino(4-chlorophenyl)(1-methyl-1H-imidazol-5-yl)methyl]-4-(3-chlorophenyl)-1-methyl-2(1H)-quinolinone, (−)-6-[amino(4-chlorophenyl)(1-methyl-1H-imidazol-5-yl)methyl]-4-(3-chloro phenyl)-1-methyl-2(1H)-quinolinone, (+)-6-[amino(4-chlorophenyl)(1-methyl-1H-imidazol-5-yl)methyl]-4-(3-chloro phenyl)-1-methyl-2(1H)-quinolinone, 5(S)-n-butyl-1-(2,3-dimethylphenyl)-4-[1-(4-cyanobenzyl)-5-imidazolylmethy 1]-2-piperazinone, (S)-1-(3-chlorophenyl)-4-[1-(4-cyanobenzyl)-5-imidazolylmethyl]-5-[2-(ethanesulfonyl)-methyl)-2-piperazinone, 5(S)-n-butyl-1-(2-methylphenyl)-4-[1-(4-cyanobenzyl)-5-imidazolylmethyl]-2-piperazinone, 1-(3-chlorophenyl) 4-[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-ylethyl)carbamoyl]piperidine, 4-{5-[4-hydroxymethyl-4-(4-chloropyridin-2-ylmethyl)-piperidin-1-ylmethyl]-2-methylimidazol-1-ylmethyl}benzonitrile, 4-{5-[4-hydroxymethyl-4-(3-chlorobenzyl)-piperidin-1-ylmethyl]-2-methylimidazol-1-ylmethyl}benzonitrile, 4-{3-[4-(2-oxo-2H-pyridin-1-yl)benzyl]-3H-imidazol-4-ylmethyl} benzonitrile, 4-{3-[4-(5-chloro-2-oxo-2H-[1,2′ ]bipyridin-5′-ylmethyl]-3H-imidazol-4-ylmethyl} benzonitrile, 4-{3-[4-(2-oxo-2H-[1,2′ ]bipyridin-5′-ylmethyl]-3H-imidazol-4-ylmethyl}benzonitrile, 4-{3-(2-oxo-1-phenyl-1,2-dihydropyridin-4-ylmethyl)-3H-imidazol-4-ylmethyl} benzonitrile, 18,19-dihydro-19-oxo-5H,17H-6,10: 12,16-dimetheno-1H-imidazo[4,3-c][1,11,4]dioxa-azacyclononadecine-9-carbonitrile, (t)-19,20-dihydro-19-oxo-5H-18,21-ethano-12,14-etheno-6, 10-metheno-22H-benzo[d]imidazo[4,3-k][1,6,9,12]-oxatriaza-cyclooctadecine-9-carbonitrile, 19,20-dihydro-19-oxo-5H, 17H-18,21-ethano-6,10: 12,16-dimetheno-22H-imidazo[3,4-h][1,8,11,14]oxatriazacyclo-eicosine-9-carbonitrile, and (±)-19,20-dihydro-3-methyl-19-oxo-5H-18,21-ethano-12,14-etheno-6,10-met heno-22H-benzo[d]imidazo[4,3-k][1,6,9,12]oxa-triazacyclooctadecine-9-carbonitrile.

Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see J. of Cancer, Vol. 35, No. 9, pp.1394-1401 (1999).

Examples of HIV protease inhibitors include amprenavir, abacavir, CGP-73547, CGP-61755, DMP-450, indinavir, nelfinavir, tipranavir, ritonavir, saquinavir, ABT-378, AG 1776, and BMS-232,632. Examples of reverse transcriptase inhibitors include delaviridine, efavirenz, GS-840, HB Y097, lamivudine, nevirapine, AZT, 3TC, ddC, and ddI. It has been reported (Nat. Med.;8(3):225-32, 2002) that HIV protease inhibitors, such as indinavir or saquinavir, have potent anti-angiogenic activities and promote regression of Kaposi sarcoma

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-i (VEGFR1) and Flk-1/KDR (VEGFR20), inhibitors of epidermal-derived, fibroblast-derived, or platelet derived growth factors, MMP (matrix metalloprotease) inhibitors, integrin blockers, interferon-oc, interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib, valecoxib, and rofecoxib (PNAS, Vol. 89, p. 7384 (1992); JNCI, Vol. 69, p. 475 (1982); Arch. Opthalmol., Vol. 108, p.573 (1990); Anat. Rec., Vol. 238, p. 68 (1994); FEBS Letters, Vol. 372, p. 83 (1995); Clin., Orthop. Vol. 313, p. 76 (1995); J. Mol. Endocrinol., Vol. 16, p.107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), carboxyamidotriazole, combretastatin A-4, squalamine, 6-O-chloroacetyl-carbonyl-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp.963-968 (October 1999); Kim et al., Nature, 362, 841-844 (1993); WO 00/44777; and WO 00/61186).

As described above, the combinations with NSAIDs are directed to the use of NSAIDs which are potent COX-2 inhibiting agents. For purposes of this specification an NSAID is potent if it possess an IC₅₀ for the inhibition of COX-2 of 1 μM or less as measured by the cell or microsomal assay known in the art.

The invention also encompasses combinations with NSAIDs which are selective COX-2 inhibitors. For purposes of this specification NSAIDs which are selective inhibitors of COX-2 are defined as those which possess a specificity for inhibiting COX-2 over COX-1 of at least 100 fold as measured by the ratio of IC₅₀ for COX-2 over ICso for COX-1 evaluated by the cell or microsomal assay disclosed hereinunder. Such compounds include, but are not limited to those disclosed in U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, U.S. Pat. No. 5,710,140, issued Jan. 20, 1998, WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999, all of which are hereby incorporated by reference. Other examples of specific inhibitors of COX-2 include those disclosed in U.S. Pat. No. 6,313,138 the disclosure of which is incorporated herein by reference in its entirety.

General and specific synthetic procedures for the preparation of the COX-2 inhibitor compounds described above are found in U.S. Pat. No. 5,474,995, issued Dec. 12, 1995, U.S. Pat. No. 5,861,419, issued Jan. 19, 1999, and U.S. Pat. No. 6,001,843, issued Dec. 14, 1999, all of which are herein incorporated by reference.

Compounds that have been described as specific inhibitors of COX-2 and are therefore useful in the present invention include, but are not limited to, the following: 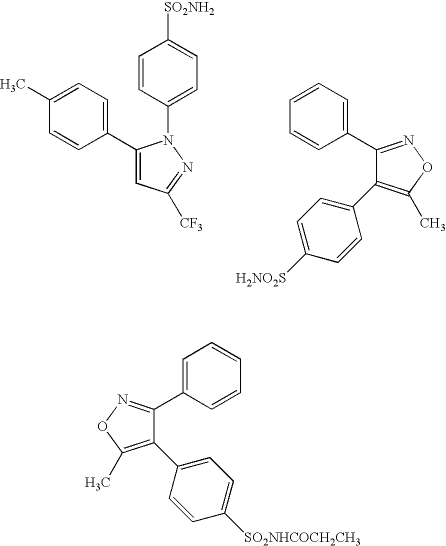
or a pharmaceutically acceptable salt thereof.

Compounds which are described as specific inhibitors of COX-2 and are therefore useful in the present invention, and methods of synthesis thereof, can be found in the following patents, pending applications and publications, which are herein incorporated by reference: WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999.

Compounds which are specific inhibitors of COX-2 and are therefore useful in the present invention, and methods of synthesis thereof, can be found in the following patents, pending applications and publications, which are herein incorporated by reference: U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, and U.S. Pat. No. 5,710,140, issued Jan. 20, 1998.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]-methyl]-1H-1,2,3-triazo 1e-4-carboxamide, CM101, squalamine, combretastatin, RP14610, NX31838, sulfated mannopentose phosphate, 7,7-(carbonyl-bis[imino-N-methyl-4,2-pyrrolocarbonyl-imino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counter-act binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆; αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁, and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-110-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, ST1571, CEP2563, 4-(3-chlorophenylamino)-5,6-dimethyl-7H-pyrrolo [2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, SU 11248, ST1571A, N-4-chlorophenyl-4-(4-pyridinylmethyl)-1-phthalazinamine, and EMD 121974.

The instant compounds are also useful, alone or in combination with platelet fibrinogen receptor (GP IIb/IIIba) antagonists, such as tirofiban, to inhibit metastasis of cancerous cells. Tumor cells can activate platelets largely via thrombin generation. This activation is associated with the release of VEGF. The release of VEGF enhances metastasis by increasing extravasation at points of adhesion to vascular endothelium (Amirkhosravi, Platelets 10, 285-292, 1999). Therefore, the present compounds can serve to inhibit metastasis, alone or in combination with GP IIb/IIIa) antagonists. Examples of other fibrinogen receptor antagonists include abciximab, eptifibatide, sibrafiban, lamifiban, lotrafiban, cromofiban, and CT50352.

If formulated as a fixed dose, such combination products employ the compounds of this invention within the dosage range described above and the other pharmaceutically active agent(s) within its approved dosage range. Compounds of the instant invention may alternatively be used sequentially with known pharmaceutically acceptable agent(s) when a combination formulation is inappropriate.

The term administration and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The compounds of the instant invention may also be co-administered with other well known therapeutic agents that are selected for their particular usefulness against the condition that is being treated. For example, the compounds of the instant invention may also be co-administered with other well known cancer therapeutic agents that are selected for their particular usefulness against the condition that is being treated. Included in such combinations of therapeutic agents are combinations of the farnesyl-protein transferase inhibitors disclosed in U.S. Pat. No. 6,313,138 and an antineoplastic agent. It is also understood that such a combination of antineoplastic agent and inhibitor of farnesyl-protein transferase may be used in conjunction with other methods of treating cancer and/or tumors, including radiation therapy and surgery.

Examples of an antineoplastic agent include, in general, microtubule-stabilizing agents (such as paclitaxel (also known as Taxol®), docetaxel (also known as Taxotere®), epothilone A, epothilone B, desoxyepothilone A, desoxyepothilone B or their derivatives; microtubule-disruptor agents; alkylating agents, anti-metabolites; epidophyllotoxin; an antineoplastic enzyme; a topoisomerase inhibitor; procarbazine; mitoxantrone; platinum coordination complexes; biological response modifiers and growth inhibitors; hormonal/anti-hormonal therapeutic agents and haematopoietic growth factors.

Example classes of antineoplastic agents include, for example, the anthracycline family of drugs, the vinca drugs, the mitomycins, the bleomycins, the cytotoxic nucleosides, the taxanes, the epothilones, discodermolide, the pteridine family of drugs, diynenes and the podophyllotoxins. Particularly useful members of those classes include, for example, doxorubicin, carminomycin, daunorubicin, aminopterin, methotrexate, methopterin, dichloro-methotrexate, mitomycin C, porfiromycin, Herceptin®, Rituxan®, 5-fluorouracil, 6-mercaptopurine, gemcitabine, cytosine arabinoside, podophyllotoxin or podo-phyllotoxin derivatives such as colchicines, etoposide, etoposide phosphate or teniposide, melphalan, vinblastine, vincristine, leurosidine, vindesine, leurosine, paclitaxel and the like. Other useful antineoplastic agents include estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, L-asparaginase, camptothecin, CPT-I 1, topotecan, ara-C, bicalutamide, flutamide, leuprolide, pyridobenzoindole derivatives, interferons and interleukins. The preferred class of antineoplastic agents is the taxanes and the preferred antineoplastic agent is paclitaxel.

Radiation therapy, including x-rays or gamma rays which are delivered from either an externally applied beam or by implantation of tiny radioactive sources, may also be used in combination with the compounds of this invention alone to treat cancer.

### EXAMPLES

The following preparations and examples are given to enable those skilled in the art to more clearly understand and to practice the present invention. They should not be considered as limiting the scope of the invention, but merely as being illustrative and representative thereof.

### Synthetic Examples

### Example 1

Synthesis of N-{4-[4-(piperidin-1-ylcarbonyl)-oxazol-2-yl]-benzyl}-2-(4-trifluoromethoxy-phenyl)-acetamide

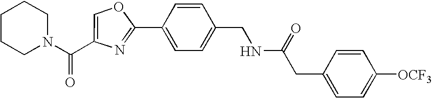

Step 1

To a stirred solution of 4-N-tert-butoxycarbonylaminomethylbenzoic acid (25.3 g, 100.7 mmol) in DMF (50 mL) was added sequentially 1-(3-dimethylaminopropyl)-3-ethylcarbodiimide hydrochloride (EDC-HCl) (23.9 g, 120.8 mmol, 1.2 eq), 1-hydroxy-benzotriazole hydrate (HOBT) (16.3 g, 120.8 mmol, 1.2 eq), N,N-diisopropylethylamine (DIPEA) (43.8 mL, 251.7 mmol, 2.5 eq), and serine methyl ester hydrochloride (18.0 g, 120.8 mmol, 1.2 eq). After stirring overnight at room temperature, the reaction mixture was diluted with water and EtOAc. The layers were separated, and the organic layer was washed successively with 1 M HCl, H₂O, saturated NaHCO₃, and brine. The organic layer was dried (Na₂SO₄), filtered, and concentrated to give 2-[4-(N-tert-butoxycarbonylamino-methyl)benzoylamino]-3-hydroxy-propionic acid methyl ester as a white solid (32.0 g, 90%).

Step 2

To a stirred solution of 2-[4-(N-tert-butoxycarbonylaminomethyl)-benzoylamino]-3-hydroxy-propionic acid methyl ester (32.0 g, 90.8 mmol) in THF (150 mL) was added Burgess Reagent [(methoxycarbonylsulfamoyl)triethyl-ammonium hydroxide, inner salt] (26.0 g, 109 mmol, 1.2 eq) and 3 A molecular sieves (1 g). The reaction mixture was allowed to stir at 60° C. for 2 hours, at which time LC showed the cyclization to be complete. After cooling to room temperature, the reaction mixture was concentrated under reduced pressure. The crude product was purified by silica gel flash chromatography [EtOAc/CH₂Cl₂ (1:1 v/v)] to give 2-[4-(N-tert-butoxycarbonylamino-methyl)-phenyl]-4,5-dihydro-oxazole-4-carboxylic acid methyl ester as a pale tan oil (29.5 g, 97%).

Step 3

To a solution of 2-[4-(N-tert-butoxycarbonylaminomethyl)-phenyl]-4,5-dihydro-oxazole-4-carboxylic acid methyl ester (25.5 g, 76.3 mmol) in CH₂Cl₂ (100 mL) was added bromotrichloromethane (BrCCl₃) (8.2 mL, 83.9 mmol, 1.1 eq) and 1,8-diazabicyclo[5.4.0]-undec-7-ene (DBU) (12.5 mL, 83.9 mmol, 1.1 eq). After stirring at room temperature overnight, the reaction mixture was concentrated and the product isolated by silica gel flash chromatography. The desired product 2-[4-(tert-butoxycarbonylaminomethyl)-phenyl]-oxazole-4-carboxylic acid methyl ester was recrystallized from MeOH as pale yellow crystals (18.4 g, 73%).

Step 4

To a solution of 2-[4-(N-tert-butoxycarbonylaminomethyl)phenyl]-oxazole-4-carboxylic acid methyl ester (13.2 g, 39.7 mmol) in CH₂Cl₂ (25 mL) was added 4 M HCl in 1,4-dioxane (49.6 mL, 5 eq.) dropwise, and the reaction mixture was allowed to stir at room temperature overnight under N₂. The desired product was precipitated as its hydrochloride salt with anhydrous ethyl ether, filtered, and dried under high vacuum to give 2-(4-aminomethyl-phenyl)-oxazole-4-carboxylic acid methyl ester hydrochloride as a white solid (10.9 g, quantitative).

Step 5

To a solution of 2-(4-aminomethylphenyl)-oxazole-4-carboxylic acid methyl ester hydrochloride (5.1 g, 22.0 mmol) in DMF (30 mL) was added EDC-HCl (5.2 g, 26.4 mmol, 1.2 eq), HOBT (3.6 g, 26.4 mmol, 1.2 eq), 4-trifluoromethoxyphenyl-acetic acid (4.8 g, 22.0 mmol, 1.0 eq), and DIPEA (9.6 mL, 54.9 mmol, 2.5 eq) at room temperature. After stirring for 2 hours, the reaction mixture was partitioned between EtOAc and water. The organic phase was washed successively with 1 M HCl, water, saturated NaHCO₃, and brine. The organic phase was dried (Na₂SO₄), filtered, and concentrated to give the desired product 2-(4-{[2-(4-trifluoromethoxy-phenyl)-acetylamino]-methyl}-phenyl)-oxazole-4-carboxylic acid methyl ester isolated as a white solid (8.1 g, 85%).

Step 6

To a solution of 2-(4-{[2-(4-trifluoromethoxyphenyl)-acetylamino]-methyl}-phenyl)-oxazole-4-carboxylic acid methyl ester (8.0 g, 18.4 mmol) in THF (150 mL), was added lithium hydroxide monohydrate (5.8 g, 92.1 mmol, 5 eq.) followed by MeOH (150 mL) and water (150 mL). After stirring at room temperature for 3 hours, the solution was acidified to pH 2-3, and partitioned between EtOAc and water. The organic layer was separated and concentrated to give the desired product 2-(4-{[2-(4-trifluoromethoxy-phenyl)-acetylamino]-methyl)}-phenyl)-oxazole-4-carboxylic acid as a white solid (5.9 g, 76%).

Step 7

To a stirred solution of 2-(4-{[2-(4-trifluoromethoxyphenyl)-acetylamino]-methyl}-phenyl)-oxazole-4-carboxylic acid (5.0 g, 11.9 mmol) in DMF (50 mL) was added benzotriazol-1-yl-oxy-trispyrrolidino-phosphonium hexafluorophosphate (PyBOP) (5.5 g, 13.1 mmol, 1.1 eq), DIPEA (4.1 mL, 23.8 mmol, 2 eq), and piperidine (2.94 mL, 29.73 mmol, 2.5 eq). After stirring at room temperature for 8 hours, the reaction was shown to be complete. The reaction mixture was then partitioned between EtOAc and water, and the organic layer was washed successively with 1 M HCl, water, saturated NaHCO₃, and brine. The organic phase was dried (Na₂SO₄), filtered and concentrated. The crude product was recrystallized from hot MeOH to give N-{4-[4-(piperidin-1-ylcarbonyl)-oxazol-2-yl]-benzyl}-2-(4-trifluoromethoxy-phenyl)-acetamide as pale yellow crystals (4.11 g, 71%). ¹H NMR (400 MHz, d₆-DMSO) δ 1.51-1.68 (m, 6H), 3.55 (s, 2H), 3.58 (brs, 2H), 3.82 (brs, 2H), 4.35 (d, 2H, J=5.6 Hz), 7.29 (brd, 2H, J=8 Hz), 7.35-7.44 (m, 4H), 7.91 (dd, 2H, J=1.6, 8.4 Hz), 8.56 (d, 1H, J=2.4 Hz), 8.68 (t, 1H, J=5.6 Hz); MS (ES) m/z 488.1 (MH⁺); MS calcd: 487.2 (M).

### Example 2

### Synthesis of N-Methyl-2-phenyl-N-{4-[4-(piperidin-1-carbonyl)-oxazol-2-yl]-benzyl} acetamide

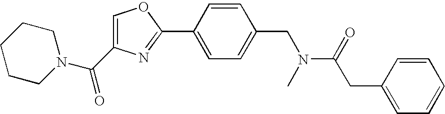

Step 1

A solution of 2-[4-(tert-butoxycarbonylamino-methyl)-phenyl]-oxazole-4-carboxylic acid methyl ester (0.085 g, 0.25 mmol) in MeOH (2.5 mL) at room temperature was treated with 1 N NaOH (0.4 mL, 0.40 mmol). After 2 hours, the reaction mixture was diluted with ethyl acetate and water. The aqueous layer was separated and carefully acidified with conc. H₃PO₄ to give a precipitate. The precipitate was filtered and dried under reduced pressure to give 2-[4-(tert-butoxycarbonylamino-methyl)-phenyl]-oxazole-4-carboxylic acid as a white powder (0.079 g, 97%).

Step 2

To a stirred solution of 2-[4-(tert-butoxycarbonylamino-methyl)-phenyl]-oxazole-4-carboxylic acid (2.1 g, 6.7 mmol) in DMF (30 mL) was added PyBOP (3.5 g, 6.7 mmol, 1.0 eq.), and DIPEA (4.7 mL, 27 mmol, 4.0 eq.). The reaction mixture was stirred at room temperature for 2 hours before adding piperidine (0.7 mL, 6.7 mmol, 1.0 eq.). After 12 hours, the reaction was determined to be complete by HPLC and LCMS. The reaction mixture was partitioned between ethyl acetate and water. The organic layer was washed successively with saturated NaCl. The organic layer was dried (MgSO₄), filtered and concentrated under reduced pressure. Purification by silica gel chromatography [CH₂Cl₂/MeOH (9:1 v/v)] followed by recrystallization from MeOH afforded {4-[4-(piperidin-1-ylcarbonyl)-oxazol-2-yl]-benzyl}-carbamic acid tert-butyl ester as a pale yellow solid (1.5 g, 58%).

Step 3

To a solution of {4-[4-(piperidin-1-ylcarbonyl)-oxazol-2-yl]-benzyl}-carbamic acid tert-butyl ester (0.5 g, 1.3 mmol) in THF (2.6 mL) and DMF (1 mL) at 0° C. under N₂ was added sodium hydride (60 wt % in oil, 100 mg, 1.9 mmol, 1.5 eq). After bubbling of the H₂ gas subsided (5 min.), methyl iodide (0.121 mL, 1.9 mmol, 1.5 eq.) was added dropwise to the reaction mixture, and the reaction mixture allowed to warm to room temperature overnight with stirring. The reaction mixture was cooled to 0° C. and quenched with MeOH (5 mL). The mixture was then partitioned between ethyl acetate and water. The organic layer was dried (MgSO₄), filtered and concentrated to give crude 2-[4-(N-methyl-N-tert-butoxycarbonylaminomethyl)-phenyl]-4-(piperidin-1-ylcarbonyl)-oxazole as a viscous brown liquid which was used in the next step without further purification.

Step 4

To a stirred solution of 2-[4-(N-methyl-N-tert-butoxycarbonylaminomethyl)-phenyl]-4-(piperidin-1-ylcarbonyl)-oxazole in EtOAc (2 mL) was added conc. hydrochloric acid (1 mL) dropwise. The reaction mixture was allowed to stir at room temperature for 2 hours. The solvent was then removed under reduced pressure to give 2-[4-(N-methylaminomethyl)-phenyl]-4-(piperidin-1-ylcarbonyl)-oxazole as the hydrochloride salt (0.25 g, 57% crude yield) which was then converted to N-methyl-2-phenyl-N-{4-[4-(piperidin-1-ylcarbonyl)-oxazol-2-yl]-benzylacetamide following similar procedure as described in Step 5, Example I above but substituting trifluoromethoxyphenylacetic acid with phenylacetic acid. MS (ES) m/z 418.4 (MH⁺); MS calcd: 417.2 (M).

### Example 3

### Synthesis of N-{4-[4-(3,3-difluoro-piperidin-1-ylcarbonyl)-oxazol-2-yl]-2-iodo-benzyl}-2,2-difluoro-2-thiophen-2-yl-acetamide

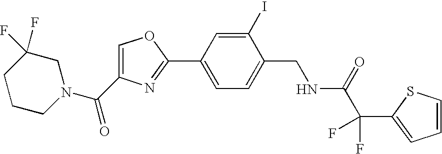

Step 1

To a stirred solution of sodium hydride (95%, 295 mg, 11.7 mmol) in DMF (40 mL) at 0° C. was added di-tert-butyl iminodicarboxylate (2.53 g, 11.7 mmol) in portions. After 1 hour, 4-bromomethyl-3-iodo-benzoic acid methyl ester (Ref: I. G. Stara, I. Stary, A. Kollarovic, F. Teply, D. Saman, P. Fiedler, Collect. Czech. Chem. Commun. 1999, 64, 649-672) (3.45 g, 9.7 mol) in DMF (20 mL) was added dropwise to the reaction mixture. The reaction mixture was then stirred at 50° C. for 4.5 hours. The reaction mixture was allowed to cool to room temperature, diluted with EtOAc (250 mL) and washed successively with saturated aqueous solution of NH₄Cl (100 mL), and brine (100 mL). The organic layer was dried (Na₂SO₄), filtered and concentrated to give a crude solid 4-[(bis-tert-butoxycarbonyl)aminomethyl]-3-iodobenzoic acid (5.85 g) which was carried onto the next step without further purification.

Step 2

A mixture of 4-[(bis-tert-butoxycarbonyl)aminomethyl]-3-iodobenzoic acid (5.85 g) and lithium hydroxide monohydrate (2.45 g, 58.3 mmol) was heated at 80° C. in THF/H₂O (2:1 v/v, 150 mL) for 9 hours. The reaction mixture was cooled to room temperature and allowed to stir overnight. Tetrahydrofuran was removed under reduced pressure followed by the addition of EtOAc (400 mL). The reaction mixture was acidified with 6 N HCl. The aqueous phase was separated and extracted with EtOAc. The combined organic extracts were dried (Na₂SO₄), filtered and concentrated to give 4-(tert-butoxycarbonylaminomethyl)-3-iodobenzoic acid as a white solid (3.44 g, 94% for 2 steps).

Step 3

Following similar procedure as in Example 1, Step 1,4-(tert-butoxycarbonyl-aminomethyl)-3-iodobenzoic acid coupled with serine methyl ester to give 2-[4-(tert-butoxycarbonylamino-methyl)-3-iodo-benzoylamino]-3-hydroxy-propionic acid methyl ester in 87% yield as foam.

Step 4

To a solution of 2-[4-(tert-butoxycarbonylamino-methyl)-3-iodo-benzoylamino]-3-hydroxy-propionic acid methyl ester (3.78 g, 7.9 mmol) in CH₂Cl₂ (25 mL) at −78° C. was added diethylaminosulfur trifluoride (DAST, 1.15 mL, 8.7 mmol) dropwise. After 1 hour, potassium carbonate (1.64 g, 11.9 mmol) was added and the reaction mixture was allowed to stir at 0° C. After 30 min., the reaction mixture was diluted with CH₂Cl₂ and washed with saturated aqueous solution of NaHCO₃ (40 mL). The aqueous was separated and extracted with CH₂Cl₂ (40 mL). The combined organic layer was washed with brine (40 mL), dried (Na₂SO₄), filtered and concentrated to give the oxazoline intermediate as foam (3.6 g). To the solution of oxazoline (3.6 g) in CH₂Cl₂ (20 mL) was added BrCCl₃ (0.86 mL, 8.7 mmol) and DBU (1.32 mL, 8.7 mmol) at room temperature. After 1 hour, the reaction mixture was filtered through a pad of silica gel and washed with CHCl₃/EtOAc (3:1 v/v, 200 mL). The solvent was concentrated under reduced pressure and precipitated with EtOAc/n-hexanes to give 2-[4-(tert-butoxycarbonylamino-methyl)-3-iodo-phenyl]-oxazole-4-carboxylic acid methyl ester, as a light yellow powder (1.98 g). The filtrate was then column chromatographed [n-hexanes/CH₂Cl₂/EtOAc (7:2:2 v/v)] to give a second batch (0.38 g). The overall yield for the cyclodehydration-oxidation step was 65%.

Step 5

Proceeding as described in Example 1, Steps 4-7, but substituting 2-[4-(N-tert-butoxycarbonylaminomethyl)-phenyl]-oxazole-4-carboxylic acid methyl ester with 2-[4-(tert-butoxycarbonylamino-methyl)-3-iodo-phenyl]-oxazole-4-carboxylic acid methyl ester in Step 4, trifluorophenylacetic acid with difluorothiophen-2-yl-acetic acid in Step 5, and piperidine with 3,3-difluoropiperidine in Step 7 provided the title compound. MS (ES) m/z 608.1 (MH⁺); MS calcd: 607.0 (M).

### Example 4

### Synthesis of 5-[4-(3,3-difluoro-piperidin-1-ylcarbonyl)-oxazol-2-yl]-2-[(2,2-difluoro-2-thiophen-2-yl-acetylamino)-methyl]-benzoic acid

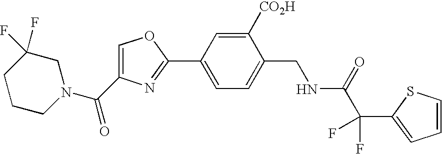

Step 1

N-{4-[4-(3,3-Difluoro-piperidin-1-ylcarbonyl)-oxazol-2-yl]-2-iodo-benzyl}-2,2-difluoro-2-thiophen-2-yl-acetamide from Example 3 could be treated further in a similar method as described in Tetrahedron Lett., 1996, 37, 5453-5456 to give 5-[4-(3,3-difluoro-piperidin-1-ylcarbonyl)-oxazol-2-yl]-2-[(2,2-difluoro-2-thiophen-2-yl-acetylamino)-methyl]-benzoic acid methyl ester.

Step 2

To a solution of 5-[4-(3,3-difluoro-piperidin-1-ylcarbonyl)-oxazol-2-yl]-2-[(2,2-difluoro-2-thiophen-2-yl-acetylamino)-methyl]-benzoic acid methyl ester (25 mg, 0.046 mmol, 1 eq) in THF (1 mL) was added lithium hydroxide (3.3 mg, 0.14 mmol, 3 eq) and water (1 mL) at room temperature. After 2 hours, the reaction mixture was concentrated under reduced pressure, diluted with water (10 mL), and acidified to pH 3 with 0.1 M HCl. The white precipitate that formed was isolated by filtration, rinsed with water and dried under reduced pressure to give 5-[4-(3,3-difluoro-piperidin-1-ylcarbonyl)-oxazol-2-yl]-2-[(2,2-difluoro-2-thiophen-2-yl-acetylamino)-methyl]-benzoic acid (16.5 mg, 68%). ¹H NMR (d₆-DMSO) δ 1.77 (br s, 2H), 2.16 (br m, 2H), 3.68 (br. M, 1H), 3.98 (br m, 2H), 4.45 (br s, 1H), 4.83 (d, 2H, J=6.7 Hz), 7.19 (m, 1H), 7.48 (m, 2H), 7.87 (d, 1H, J=4 Hz), 8.16 (d, 1H, J=5.2 Hz), 8.48 (s, 1H), 8.75 (s, 1H), 9.63 (t, 1H, J=6.7 Hz), 12.78 (br s, 1H); MS (ES) m/z 526.0 (MH⁺); MS calcd: 525.1 (M).

### Example 5

### Synthesis of 3-{5-[4-(3,3-difluoro-piperidin-1-ylcarbonyl)-oxazol-2-yl]-2-[(2,2-difluoro-2-thiophen-2-yl-acetylamino)-methyl]-phenyl}-acrylic acid methyl ester

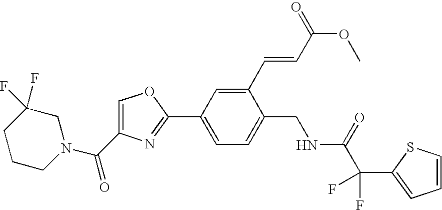

Step 1

To a solution of N-{4-[4-(3,3-difluoro-piperidin-1-ylcarbonyl)-oxazol-2-yl]-2-iodo-benzyl}-2,2-difluoro-2-thiophen-2-yl-acetamide (96 mg, 0.16 mmol, 1 eq) from Example 4 in DMF (2 mL) under N₂ was added methyl acrylate (0.028 mL, 0.32 mmol, 2 eq), and triethylamine (0.044 mL, 0.32 mmol, 2 eq). A solution of palladium acetate (3.6 mg, 0.016 mmol, 0.1 eq) and tri-o-tolylphosphine (9.7 mg, 0.032 mmol, 0.2 eq) in DMF (0.79 mL) was prepared and introduced into the reaction mixture. The reaction mixture was then heated at 100° C. for 12 hours. The cooled reaction mixture was concentrated under reduced pressure, and purified by silica gel chromatography [hexanes/EtOAc (1:1 v/v) to (1:2 v/v)]. The desired product 3-{5-[4-(3,3-difluoro-piperidin-1-ylcarbonyl)-oxazol-2-yl]-2-[(2,2-difluoro-2-thiophen-2-yl-acetylamino)-methyl]-phenyl}-acrylic acid methyl ester was obtained as a yellow solid (0.052 g, 57%). ¹H-NMR (d₆-DMSO) δ 1.75 (br m, 2H), 2.10 (m, 2H), 3.53 (br m, 1H), 3.73 (s, 3H), 3.95 (br m, 2H), 4.40 (br m, 1H), 4.55 (d, 1H, J=5.2 Hz), 6.60 (d, 1H, J=16 Hz), 7.12 (dt, 1H, J=4, 1.2 Hz), 7.40 (m, 1H), 7.47 (d, 1H, J=8.4 Hz), 7.81 (dd, 1H, J=5.2, 1.2 Hz), 8.00-7.96 (m, 2H), 8.19 (d, 1H, J=1.6 Hz), 8.72 (s, 1H), 9.68 (t, 1H, J=6 Hz); MS (ES) m/z 566.1 (MH⁺); MS calcd: 565.1 (M).

### Example 6

### Synthesis of 3-{5-[4-(3,3-difluoro-piperidin-1-ylcarbonyl)-oxazol-2-yl]-2-[(2,2-difluoro-2-thiophen-2-yl-acetylamino)-methyl]-phenyl}-propionic acid

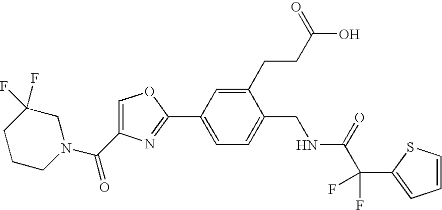

Step 1

To a solution of 3-{5-[4-(3,3-difluoro-piperidin-1-ylcarbonyl)-oxazol-2-yl]-2-[(2,2-difluoro-2-thiophen-2-yl-acetylamino)-methyl]-phenyl}-acrylic acid methyl ester (0.052 g, 0.092 mmol) from Example 5 in MeOH/THF (4:1 v/v, 12 mL) was added palladium on carbon (10%, 20 mg). The reaction mixture was shaked under H₂ at 50 psi using a Parr hydrogenator. The catalyst was filtered and the solvent removed under reduced pressure. The desired product 3-{5-[4-(3,3-difluoro-piperidin-1-ylcarbonyl)-oxazol-2-yl]-2-[(2,2-difluoro-2-thiophen-2-yl-acetylamino)-methyl]-phenyl}-propionic acid methyl ester was obtained as clear oil (0.052 g, quantitative).

Step 2

To a solution of 3-{5-[4-(3,3-difluoro-piperidin-1-ylcarbonyl)-oxazol-2-yl]-2-[(2,2-difluoro-2-thiophen-2-yl-acetylamino)-methyl]-phenyl}-propionic acid methyl ester (0.045 g, 0.079 mmol, 1 eq) in THF (2 mL) was added lithium hydroxide (0.017 g, 0.39 mmol, 5 eq) and water (1 mL) at room temperature. After 2 hours, the reaction mixture was concentrated under reduced pressure, diluted with water (10 mL), and acidified to pH 3 with 4 M HCl. The aqueous layer was extracted with EtOAc (10 mL) which was separated and dried (Na₂SO₄). The residue was purified by reversed phase HPLC (2 to 50% acetonitrile/water, 0.1% HCl, 50 mL/min), to give 3-{5-[4-(3,3-difluoro-piperidin-1-ylcarbonyl)-oxazol-2-yl]-2-[(2,2-difluoro-2-thiophen-2-yl-acetylamino)-methyl]-phenyl}-propionic acid (0.010 g, 23%) as a white solid.

¹H NMR (d₆-DMSO) δ 1.75 (br m, 2H), 2.15 (br m, 2H), 2.59 (t, 2H, J=7.6 Hz), 3.00 (t, 2H, J=7.6 Hz), 3.67 (br m, 1H), 3.95 (br m, 2H), 4.5 (m, 3H, 6 Hz), 7.18 (m, 1H), 7.38-7.37 (m, 1H), 7.46-7.45 (m, 1H), 7.86-7.84 (m, 2H), 8.71 (s, 1H), 9.69 (t, 1H, J=6 Hz); MS (ES) m/z 554.3 (MH⁺); MS calcd: 553.1 (M).

### Example 7

### Synthesis of N-{3,5-difluoro-4-[4-(piperidin-1-ylcarbonyl)-oxazol-2-yl]-benzyl}-2-phenyl-acetamide

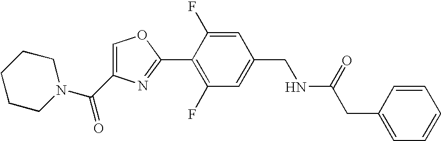

Step 1

To a suspension of lithium borohydride (60 mg, 2.73 mmol) in TIIF (10 mL) was added 4-cyano-2,6-difluoro-benzoic acid (Ref: M. J. Fisher, et al, Bioorg. Med. Chem. Lett., 2000, 385-390) (200 mg, 1.09 mmol) under N₂. After 1 hour, the reaction mixture was quenched with 1 N HCl followed by concentration of THF. The crude amine was carried onto the next step without further purification. The pH of the amine solution was adjusted to 10 with the addition of 1 N NaOH. 1,4-Dioxane (10 mL) and di-tert-butyl dicarbonate (476 mg, 2.18 mmol) was added to the reaction mixture. Upon completion, the solution was acidified with 1 M KHSO₄ followed by extraction with EtOAc. The organic extract was dried (Na₂SO₄), filtered and concentrated to give the 4-(tert-butoxycarbonyl-amino-methyl)-2,6-difluoro-benzoic acid as a white solid (290 mg, 92%).

Step 2

Following similar procedure as in Example 1, Step 1,4-(tert-butoxycarbonyl-amino-methyl)-2,6-difluoro-benzoic acid coupled with serine methyl ester to give 2-[4-(tert-butoxycarbonylamino-methyl)-2,6-difluoro-benzoylamino]-3-hydroxy-propionic acid methyl ester as a white solid.

Step 3

2-[4-(tert-Butoxycarbonyl-amino-methyl)-2,6-difluoro-benzoylamino]-3-hydroxy-propionic acid methyl ester was further treated as in Example 3, Steps 4-5 to give N-{3,5-difluoro-4-[4-(piperidin-1-ylcarbonyl)-oxazol-2-yl]-benzyl}-2-phenyl-acetamide. ¹H NMR (400 MHz, d₆-DMSO) δ 1.50-1.67 (m, 6H), 3.53 (s, 2H), 3.58 (brs, 2H), 3.79 (brs, 2H), 4.35 (d, 2H, J=5.9 Hz), 7.14 (d, 2H, J=9.8 Hz), 7.21-7.34 (m, 5H), 8.70 (t, 1H, J=5.9 Hz), 8.73 (s, 1H); MS (ES) m/z 440.1 (MH⁺); MS calcd: 439.2 (M).

### Example 8

### Synthesis of N-{4-[4-(3,3-difluoro-piperidin-1-ylcarbonyl)-oxazol-2-yl]-2-nitro-benzyl}-2-phenyl-acetamide

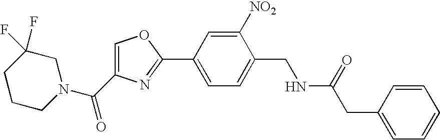

Step 1

To a solution of 4-aminomethylbenzoic acid (5.0 g, 33.1 mmol, 1 eq) in trifluoroacetic acid (50 mL) at room temperature was added NaNO₃ (3.09 g, 36.4 mmol, 1.1 eq) in portions. Conc. H₂SO₄ (20 mL) was added gradually over 10 min., using an ice bath for temperature control. The reaction mixture was allowed to stir for 2 hours, and then poured carefully into ethyl ether (600 mL) to give a yellow precipitate. The ether was decanted from the oily precipitate, which was washed further with ether, and dried under high vacuum. The yellow solid 4-aminomethyl-3-nitro-benzoic acid dihydrogen sulfate was taken onto the next step without further purification.

Step 2

A solution of the 4-aminomethyl-3-nitro-benzoic acid dihydrogen sulfate in water (200 mL) was treated with potassium carbonate to adjust the pH to 10. Di-tert-butyl-dicarbonate (8.67 g, 39.7 mmol, 1.2 eq) was added and the reaction mixture allowed to stir for 12 hours. The solution was acidified carefully with 4 M HCl to pH 3, and partitioned between chloroform and water. The organics were concentrated under reduced pressure, and the residue was purified by silica gel chromatography [CHCl₃/MeO/HOAc (88/10/2 v/v)] to give 4-(tert-butoxycarbonylamino-methyl)-3-nitro-benzoic acid as a white crystalline solid (4.80 g, 49%).

Step 3

4-(tert-Butoxycarbonylamino-methyl)-3-nitro-benzoic acid was further treated as in Example 3 to give N-{4-[4-(3,3-difluoro-piperidin-1-ylcarbonyl)-oxazol-2-yl]-2-nitro-benzyl}-2-phenyl-acetamide. ¹H NMR (d₆-DMSO) δ 1.75 (br m, 2H), 2.12 (m, 2H), 3.53 (br m, 2H), 3.65 (br m, 1H), 3.95 (br m, 2H), 4.40 (br m, 1H), 4.60 (d, 2H, J=6 Hz), 7.33-7.23 (m, 5H), 7.66 (d, 1H, J=8.4 Hz), 8.24 (dd, 1H, J=8.4, 2.0 Hz), 8.50 (d, 1H, J=2 Hz), 8.70 (t, 1H, J=5.6 Hz), 8.78 (s, 1H); MS (ES) m/z 485.1 (MH⁺); MS calcd: 484.2 (M).

### Example 9

### Synthesis of N-{2-amino-4-[4-(3,3-difluoro-piperidin-1-ylcarbonyl)-oxazol-2-yl]-benzyl}-2-phenyl-acetamide

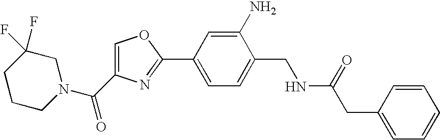

Step 1

A solution of N-{4-[4-(3,3-difluoro-piperidin-1-ylcarbonyl)-oxazol-2-yl]-2-nitro-benzyl}-2-phenyl-acetamide from Example 8 (0.17 g, 0.35 mmol) in THF (10 mL) and HOAc (5 drops) was hydrogenated in the presence of palladium on charcoal (10%, 20 mg) for 2 days under 1 atm of H₂. The catalyst was filtered and the solvent removed under reduced pressure. The residue was purified by reversed phase HPLC (2 to 50% acetonitrile/water, 0.1% HCl, 50 mL/min), to give N-{2-amino-4-[4-(3,3-difluoro-piperidin-1-ylcarbonyl)-oxazol-2-yl]-benzyl}-2-phenyl-acetamide as a white amorphous powder. ¹H NMR (d₆-DMSO) δ 1.75 (br m, 2H), 2.10 (br m, 2H), 3.48 (s, 2H), 3.65 (br m, 1H), 3.95 (br m, 2H), 4.15 (d, 2H, J=6 Hz), 4.45 (br m, 1H), 5.44 (br s, 2H), 7.32-7.10 (m, 8H), 8.53 (t, 1H, J=6.4 Hz), 8.68 (s, 1H); MS (ES) m/z 455.1 (MH⁺); MS calcd: 454.2 (M).

### Example 10

### Synthesis of N-{3-methyl-4-[4-(piperidin-1-ylcarbonyl)-oxazol-2-yl]-benzyl}-2-phenyl-acetamide

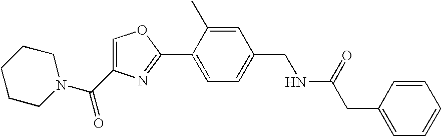

Step 1

To a refluxing solution of 2,4-dimethyl-benzoic acid methyl ester (10 g, 60.9 mmol) in CCl₄ (50 mL) was added a mixed sample of N-bromosuccinimide (11.6 g, 65.2 mmol) and benzoyl peroxide (100 mg, 0.41 mmol) via a powder additional funnel over 45 min. After addition of reagents, the funnel was rinsed with CCl₄ (25 mL) and the reaction mixture was allowed to stir for an additional 3.5 hours. The reaction mixture was then cooled to room temperature and the precipitate was filtered and rinsed with CCl₄ (50 mL). The solvent was concentrated under reduced pressure and the crude oil chromatographed [EtOAc/n-hexanes (2:98 v/v) to (4:96)] to give an inseparable mixture of bromides (8.4 g, 57%) as oil. The ratio of the desired 4-bromomethyl-2-methyl-benzoic acid methyl ester and its regioisomer 2-bromomethyl-4-methyl-benzoic acid methyl ester was 1:1.25.

Step 2

To a solution of di-tert-butyl imino dicarboxylate (8.2 g, 37.8 mmol) in DMF (80 mL) at 0° C. was added sodium hydride (95%, 956 mg, 37.8 mmol) in one portion. After 20 min., a solution of the bromides (7.36 g, 30.3 mmol) from Step 1 in DMF (40 mL) was added dropwise. The reaction mixture was then heated at 50° C. for 5 hours. After cooling to room temperature, the reaction mixture was diluted with EtOAc (400 mL) and washed with a saturated solution of NH₄Cl (400 mL), and brine (400 mL). The organic extract was dried (Na₂SO₄), filtered through a short pad of silica gel, and concentrated to give yellow oil (13.1 g). The mixture of bis-Boc amine was carried onto the next step without further purification.

To a solution of crude bis-Boc amine (13.1 g) in THF (240 mL) was added a solution of lithium hydroxide monohydrate (8.7 g, 207 mmol) in water (120 mL). After 1 hour, the reaction mixture was heated to 70° C. After 24 hours, the reaction mixture was cooled to room temperature followed by the removal of THF under reduced pressure. The aqueous solution was acidified with 6 N HCl followed by extraction with EtOAc (3×300 mL). The organic extract was dried (Na₂SO₄), filtered, and concentrated to give crude 4-(tert-butoxycarbonylamino-methyl)-2-methyl-benzoic acid together with its regioisomer (8.6 g).

Step 3

Following similar procedure as in Example 1, Step 1, crude 4-(tert-butoxycarbonylamino-methyl)-2-methyl-benzoic acid was coupled to serine methyl ester. The crude product was chromatographed [EtOAc/n-hexanes (5:4 v/v) to (6:1 v/v)] to give first 2-[2-(tert-butoxycarbonylamino-methyl)-4-methyl-benzoylamino]-3-hydroxy-propionic acid methyl ester (4.2 g, 37% over 2 steps) as foam followed by the desired product 2-[4-(tert-butoxycarbonylamino-methyl)-2-methyl-benzoylamino]-3-hydroxy-propionic acid methyl ester (3.2 g, 28% over 2 steps) as a white solid.

Step 4
2-[4-(tert-Butoxycarbonylamino-methyl)-2-methyl-benzoylamino]-3-hydroxy-propionic acid methyl ester was further treated as in Example 3, Steps 4-5 to give N-{3-methyl-4-[4-(piperidin-1-ylcarbonyl)-oxazol-2-yl]-benzyl}-2-phenyl-acetamide. ¹H NMR (400 MHz, d₆-DMSO) δ 1.52-1.68 (m, 6H), 3.50 (s, 2H), 3.60 (brs, 2H), 3.86 (brs, 2H), 4.30 (d, 2H, J=5.9 Hz), 7.19-7.32 (m, 7H), 7.85 (d, 1H, J=8.2 Hz), 8.59 (s, 1H), 8.63 (t, 1H, J=5.9 Hz); MS (ES) m/z 418.3 (MH⁺); MS calcd: 417.2 (M).

### Example 11

### Synthesis of N-{2-hydroxy-4-[4-(piperidin-1-ylcarbonyl)-oxazol-2-yl]-benzyl}-2-phenyl-acetamide

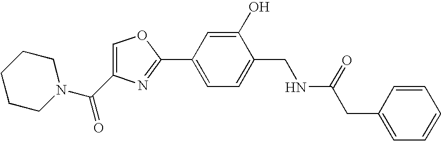

Step 1

A solution of 4-bromomethyl-3-methoxybenzoic acid methyl ester (650 mg, 2.5 mol) and hexamethylenetetramine (400 mg, 2.8 mmol, 1.12 eq) was stirred in CHCl₃ (10 mL) at room temperature. After 3 days, LCMS showed complete conversion to the hexamethylene-tetramine adduct, MS (ES) m/z 319.38 (M+); MS calcd: 319.2 (M). Petroleum ether (40 mL) was added, and the resulting white solid was collected by filtration. To the solid was added ethanol (20 mL) and conc. HCl (1.5 mL, 18 mmol) and the reaction mixture heated to reflux. After 4 hours, the reaction mixture was cooled to room temperature, and MeOH was added to dissolve the resulting solid. The solution was concentrated, taken up in MeOH again and concentrated, then triturated with hexane to give crude 4-aminomethyl-3-methoxy-benzoic acid methyl ester hydrochloride (1.04 g) which was used for the next step without further purification.

Step 2

To a solution of 4-aminomethyl-3-methoxybenzoic acid methyl ester hydrochloride (300 mg crude material) in DMF (3 mL) was added phenylacetylchloride (0.155 mL, 1.17 mmol) and DIPEA (0.55 mL, 3.18 mmol). The reaction mixture was allowed to stir at room temperature for 3 hours. The reaction mixture was partitioned between EtOAc (30 mL) and 1 N HCl (20 mL), and the organic layer washed with water (20 mL), saturated NaHCO₃, and brine, The extract was dried (Na₂SO₄), filtered, and concentrated to give 3-methoxy-4-(phenylacetylaminomethyl)-benzoic acid methyl ester (242 mg, 0.77 mmol).

Step 3

To a solution of 3-methoxy-4-(phenylacetylaminomethyl)-benzoic acid methyl ester (242 mg, 0.77 mmol) in THF (5 mL) was added aq. LiOH solution (1 M, 3.0 mL, 3.0 mmol). After 2 hours, the reaction mixture was acidified with 2 N HCl to pH 2, and concentrated to give a white solid. The reaction mixture was partitioned between EtOAc (30 mL) and water (20 mL), and the organic layer was washed with brine (20 mL), dried (Na₂SO₄), filtered, and concentrated to give 3-methoxy-4-(phenylacetylamino-methyl)-benzoic acid which was used for the next step without further purification.

Step 4

Following similar procedure as in Example 1, Step 1,3-methoxy-4-(phenylacetyl-amino-methyl)-benzoic acid coupled with serine methyl ester to give 3-hydroxy-2-[3-methoxy-4-(phenylacetylaminomethyl)-benzoylamino]-propionic acid methyl ester (68% over 2 steps).

Step 5

Following similar procedure as in Example 3, Step 4,3-hydroxy-2-[3-methoxy-4-(phenylacetylaminomethyl)-benzoylamino]-propionic acid methyl ester underwent cyclodehydration-oidation to give 2-[3-methoxy-4-(phenylacetylamino-methyl)-phenyl]-oxazole-4-carboxylic acid methyl ester in 86% yield.

Step 6

To a solution of 2-[3-methoxy-4-(phenylacetylaminomethyl)-phenyl]-oxazole-4-carboxylic acid methyl ester (50 mg, 0.13 mmol) in CH₂Cl₂ (3 mL) was added boron tribromide (1 M solution in CH₂Cl₂, 1.97 mL, 1.97 mmol, 15 eq), and the solution was allowed to stir at room temperature. After 2 hours, the reaction mixture was diluted with 1N HCl (10 mL). The aqueous phase was separated and extracted with ethyl acetate (30 mL). The combined organic extracts were washed with water (20 mL) and brine (20 mL), dried (Na₂SO₄), filtered, and concentrated to give the desired product 2-[3-hydroxy-4-(phenylacetylaminomethyl)-phenyl]-oxazole-4-carboxylic acid (48 mg).

Step 7

Following similar procedure as in Example. 1, Step 7, 2-[3-hydroxy-4-(phenyl-acetylaminomethyl)-phenyl]-oxazole-4-carboxylic acid reacted with piperidine using PyBroP coupling method to give N-{2-hydroxy-4-[4-(piperidin-1-ylcarbonyl)-oxazol-2-yl]-benzyl}-2-phenylacetamide. MS (ES) m/z 420.2 (MH⁺); MS calcd: 419.2 (M). ¹H NMR: (400 MHz, DMSO-d₆) δ 10.06 (s, 1H), 8.52 (s, 1H), 8.48 (t, 1H, J=5.6 Hz), 7.41 (d, 1H, J=1.6 Hz), 7.34 (dd, 1H, J=8.4, 1.6 Hz), 7.30-7.17 (m, 6H), 4.23 (d, 2H, J=6 Hz), 3.81 (br m, 2H), 3.56 (br m, 2H), 3.49 (s, 2H), 1.63 (m, 2H), 1.55 (m, 4H);

### Example 12

### Synthesis of N-{2-Methoxy-4-[4-(piperidin-1-ylcarbonyl)-oxazol-2-yl]-benzyl}-2-phenyl-acetamide

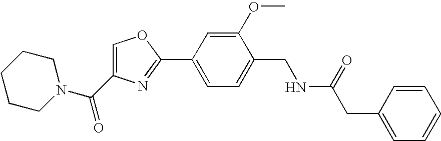

Step 1

To a solution of 2-[3-methoxy-4-(phenylacetylaminomethyl)-phenyl]-oxazole-4-carboxylic acid methyl ester (Example 11, Step 5) in MeOH/THF/H₂O (1:1:1 v/v, 9 mL) was added aq. LiOH solution (1 M, 2.0 mL, 2.0 mmol, 5 eq.). The reaction mixture was stirred overnight and acidified with 2N HCl to pH 2. The reaction mixture was then diluted with EtOAc (30 mL) and washed with brine (20 mL), dried (Na₂SO₄), filtered, and concentrated to give 2-[3-methoxy-4-(phenylacetylamino-methyl)-phenyl]-oxazole-4-carboxylic acid (100 mg, 69%) as a cream solid.

Step 2

Following similar procedure as in Example 1, Step 7, 2-[3-methoxy-4-(phenyl-acetylamino-methyl)-phenyl]-oxazole-4-carboxylic acid reacted with piperidine using PyBOP coupling method to give N-{2-methoxy-4-[4-(piperidin-1-ylcarbonyl)-oxazol-2-yl]-benzyl}-2-phenylacetamide (67%) as cream colored crystals. ¹H NMR (400 MHz, d₆-DMSO) δ 1.50-1.67 (m, 6H), 3.51 (s, 2H), 3.58 (brs, 2H), 3.80 (brs, 2H), 3.89 (s, 3H), 4.27 (d, 2H, J=5.9 Hz), 7.21-7.33 (m, 5H), 7.26 (d, 1H, J=7.8 Hz), 7.49 (d, 1H, J=1.2 Hz), 7.52 (dd, 1H, J=1.2, 7.8 Hz), 8.47 (t, 1H, J=5.9 Hz), 8.58 (s, 1H); MS (ES) m/z 434.2 (MH⁺); MS calcd: 433.2 (M).

### Example 13

### Synthesis of N-{3-methoxy-4-[4-(piperidin-1-ylcarbonyl)-oxazol-2-yl]-benzyl}-2-phenyl-acetamide

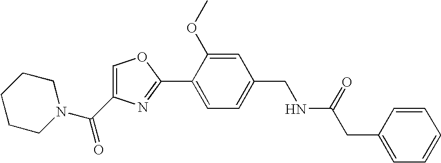

Step 1

Following similar procedure as in Example 3, Steps 1-5, N-{3-methoxy-4-[4-(piperidin-1-ylcarbonyl)-oxazol-2-yl]-benzyl}-2-phenyl-acetamide can be obtained using 4-bromomethyl-2-methoxy-benzoic acid methyl ester (M. Julia, F. Chastrette, Bull. Soc. Chim. Soc., 1962, 2255-2261) as starting material. ¹H NMR (400 MHz, d₆-DMSO) δ 1.50-1.67 (m, 6H), 3.50 (s, 2H), 3.58 (brs, 2H), 3.76 (s, 3H), 3.83 (brs, 2H), 4.34 (d, 2H, J=5.9 Hz), 6.94 (dd, 1H, J=1.2, 7.8 Hz), 7.00 (brs, 1H), 7.21-7.32 (m, 5H), 7.75 (d, 1H, J=7.8 Hz), 8.55 (s, 1H), 8.67 (t, 1H, J=5.9 Hz); MS (ES) m/z 433.7 (MH⁺); MS calcd: 433.2 (M).

### Example 14

### Synthesis of N-{3-hydroxy-4-[4-(piperidin-1-ylcarbonyl)-oxazol-2-yl]-benzyl}-2-phenyl-acetamide

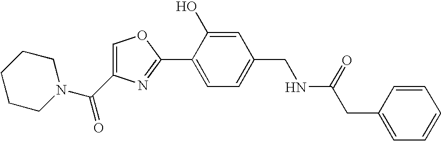

Step 1

Following similar procedure as in Example 11, Step 6, N-{3-methoxy-4-[4-(piperidin-1-ylcarbonyl)-oxazol-2-yl]-benzyl}-2-phenyl-acetamide underwent demethylation with boron tribromide to give N-{3-hydroxy-4-[4-(piperidin-1-ylcarbonyl)-oxazol-2-yl]-benzyl}-2-phenyl-acetamide. ¹H NMR (400 MHz, d₆-DMSO) δ 1.51-1.68 (m, 6H), 3.50 (s, 2H), 3.58 (brs, 2H), 3.70 (brs, 2H), 4.28 (d, 2H, J=5.9 Hz), 6.88 (dd, 1H, J=1.6, 8.2 Hz), 6.93 (s, 1H), 7.21-7.33 (m, 5H), 7.76 (d, 1H, J=8.2 Hz), 8.62 (t, 1H, J=5.9 Hz), 8.62 (s, 1H), 10.54 (brs, 1H); MS (ES) m/z 420.1 (MH⁺); MS calcd: 419.2 (M).

### Example 15

### Synthesis of N-{4-[4-(piperidin-1-ylcarbonyl)-thiazol-2-yl]-benzyl}-2-phenylacetamide

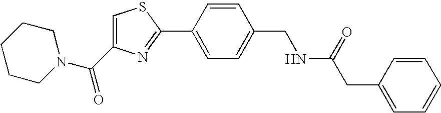

Step 1

A solution of 2-[4-(N-tert-butoxycarbonylaminomethyl)-benzoylamino]-3-hydroxy-propionic acid methyl ester (105.7 mg, 0.3 mmol) and Lawesson's Reagent [2,4-bis(4-methoxyphenyl)-1,3-dithia-2,4-diphosphetane-2,4-disulfidel (133.5 mg, 0.33 mmol) was refluxed in toluene (10 mL). After 40 min., the reaction mixture was cooled and concentrated under reduced pressure. The crude syrup was column chromatographed [CHCl₃/MeOH (96:4 v/v)] to give 2-[4-(N-tert-butoxycarbonylamino-methyl)-phenyl]-4,5-dihydro-thiazole-4-carboxylic acid methyl ester (64.7 mg, 61%) as an oil.

Step 2

To a stirred solution of 2-[4-(N-tert-butoxycarbonylamino-methyl)-phenyl]-4,5-dihydro-thiazole-4-carboxylic acid methyl ester (62 mg, 0.1769 mmol) in CH₂Cl₂ was added BrCCl₃ (19.2 μL, 0.1946 mmol) and DBU (29.1 μL, 0.1946 mmol) at room temperature. After 1.5 hours, the reaction mixture was concentrated and column chromatographed [n-hexane/EtOAc (3:2 v/v)] to give 2-[4-(N-tert-butoxycarbonyl-aminomethyl)-phenyl]-thiazole-4-carboxylic acid methyl ester (51.1 mg, 83%) as an oil.

Step 3

Proceeding as described in Example 1, Steps 4-7, but substituting 2-[4-(N-tert-butoxycarbonylaminomethyl)-phenyl]-oxazole-4-carboxylic acid methyl ester with 2-[4-(N-tert-butoxycarbonylaminomethyl)-phenyl]-thiazole-4-carboxylic acid methyl ester in Step 4 and trifluorophenylacetic acid with phenylacetic acid in Step 5, provided the title compound.

¹H NMR (400 MHz, d₆-DMSO) δ 1.51-1.68 (m, 6H), 3.49 (2, 2H), 3.59 (brs, 4H), 4.31 (d, 2H, J=5.9 Hz), 7.18-7.32 (m, 5H), 7.35 (d, 2H, J=8.2 Hz), 7.87 (d, 2H, J=8.2 Hz), 8.04 (s, 1H), 8.62 (t, 1H, J=5.9 Hz); MS (ES) m/z 420.2 (MH⁺); MS calcd: 419.2 (M).

### Example 16

### Synthesis of N-{4-[5-methyl-4-(piperidin-1-ylcarbonyl)-oxazol-2-yl]-benzyl}-2-phenyl-acetamide

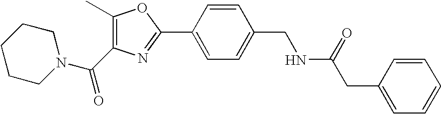

Step 1

Following the same procedure as in Example 1, Step 1, threonine methyl ester was coupled with 4-N-tert-butoxycarbonylaminomethylbenzoic acid to give 2-[4-(tert-butoxycarbonylamino-methyl)-benzoylamino]-3-hydroxy-butyric acid methyl ester in 84% yield as a white solid.

Step 2

To a solution of 2-[4-(tert-butoxycarbonylamino-methyl)-benzoylamino]-3-hydroxy-butyric acid methyl ester (4.0 g, 10.9 mmol) in CH₂Cl₂ (50 mL) at −20° C. was added [bis(2-methoxyethyl)amino]sulfur trifluoride (Deoxo-Fluor®) (2.3 mL, 12.6 mmol) dropwise. After 1 hour, a saturated aqueous solution of Na₂CO₃ (10 mL) was added at −10° C. and the reaction mixture was allowed to stir for an hour. The reaction mixture was then diluted with CH₂Cl₂ and washed with saturated aqueous solution of Na₂CO₃ (2×60 mL) and brine (50 mL). The organic phase was dried (Na₂SO₄), filtered and concentrated to give the intermediate oxazoline as foam (4.3 g). To a solution of the crude oxazoline (4.3 g) in CH₂Cl₂ (50 mL) was added BrCCl₃ (1.2 mL, 12 mmol) and DBU (1.8 mL, 12 mmol) at room temperature. After 1.5 hours, the reaction mixture was filtered through a pad of silica gel and washed with CHCl₃/MeOH (9:1 v/v, 50 mL). The filtrate was concentrated under reduced pressure and column chromatographed [n-hexane/EtOAc (5:4 v/v)] to give 2-[4-(tert-butoxycarbonylamino-methyl)-phenyl]-5-methyl-oxazole-4-carboxylic acid methyl ester (3.1 g, 89%) as a white solid.

Step 3

Proceeding as in Example 1, Steps 4-7, but substituting 2-[4-(N-tert-butoxycarbonyl-aminomethyl)-phenyl]-oxazole-4-carboxylic acid methyl ester with 2-[4-(tert-butoxycarbonylamino-methyl)-phenyl]-5-methyl-oxazole-4-carboxylic acid methyl ester in Step 4 and trifluorophenylacetic acid with phenylacetic acid in Step 5, provided the title compound. ¹H NMR (400 MHz, d₆-DMSO) δ 1.50-1.66 (m, 6H), 3.50 (d, check), 3.57 (brs, 2H), 3.73 (brs, 2H), 4.33 (d, 2H, J=5.9 Hz), 7.21-7.33 (m, 5H), 7.37 (d, 2H, J=8.2 Hz), 7.87 (d, 2H, J=8.2 Hz), 8.64 (t, 1H, J=5.9 Hz); MS (ES) m/z 418.4 (MH⁺); MS calcd: 417.2 (M).

### Example 17

### Synthesis of 2-phenyl-N-{4-[4-(piperidin-1-ylcarbonyl)-pyrazol-1-yl]-benzyl}-acetamide

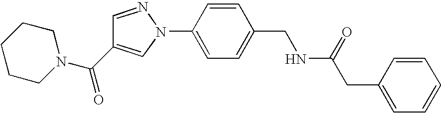

Step 1

To a solution of ethoxycarbonylmalonaldehyde (ref: S. Torii, T. Inokuchi, M. Kubota, Synthesis, 1986, 400-402) (1.4 g, 9.71 mmol) in EtOH (10 mL) at 0° C. was added dropwise a suspension of 4-cyanophenyl hydrazine hydrochloride in EtOH (60 mL). The reaction mixture was stirred at room temperature for 1 day. The yellow precipitate was filtered and dried under high vacuum to give 1-(4-cyano-phenyl)-1H-pyrazole-4-carboxylic acid ethyl ester (1.33 g, 68%).

Step 2

A suspension of 1-(4-cyano-phenyl)-1H-pyrazole-4-carboxylic acid ethyl ester (467 mg, 1.94 mmol) and palladium on carbon (10%, 100 mg) in CHCl₃/MeOH/conc. HCl (55 mL, 8:2:1 v/v) was stirred under H₂ at atmospheric pressure for 17 hours. The catalyst was filtered through Celite and washed with MeOH. The solvent was concentrated to give 1-(4-aminomethyl-phenyl)-1H-pyrazole-4-carboxylic acid ethyl ester hydrochloride as a white solid (552 mg, quantitative).

Step 3

Proceeding as described in Example 1, Steps 5-7 above, but substituting 2-(4-aminomethylphenyl)-oxazole-4-carboxylic acid methyl ester hydrochloride with 1-(4-aminomethyl-phenyl)-1H-pyrazole-4-carboxylic acid ethyl ester hydrochloride and trifluorophenylacetic acid with phenylacetic acid in Step 5, provided the title compound. ¹H NMR (400 MHz, d₆-DMSO) δ 1.50-1.66 (m, 6H), 3.49 (s, 2H), 3.54-3.61 (m, 4H), 4.31 (d, 2H, J=5.9 Hz), 7.20-7.33 (m, 5H), 7.35 (d, 2H, J=8.6 Hz), 7.81 (d, 2H, J=8.6 Hz), 7.91 (s, 1H), 8.61 (t, 1H, J=5.9 Hz), 8.73 (s, 1H); MS (ES) m/z 403.1 (MH⁺); MS calcd: 402.2 (M).

### Example 18

### Synthesis of N-{4-[5-(3,3-difluoro-piperidin-1-ylcarbonyl)-1H-imidazol-2-yl]-benzyl}-2-phenyl-acetamide

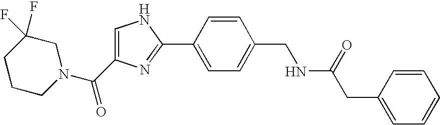

Step 1

To a solution of 4-cyanobenzyl bromide (7.53 g, 38.4 mmol, 1 eq) in DMF (200 mL) was added potassium phthalimide (7.11 g, 38.4 mmol, 1 eq) at room temperature. After stirring for 2 hours, another portion of potassium phthalimide (2.0 g, 10.8 mmol, 0.3 eq) was added and stirring maintained for 12 hours. The reaction mixture was diluted with water (100 mL) and stirring continued for 5 min. The resulting precipitate was filtered, rinsed with water, and dried under high vacuum to give 4-(1,3-dioxo-1,3-dihydro-isoindol-2-ylmethyl)-benzonitrile as a fluffy white powder (8.91 g, 88%).

Step 2

Hydrogen chloride gas was introduced as a gentle stream to the point of saturation in a heterogeneous suspension of 4-(1,3-dioxo-1,3-dihydro-isoindol-2-ylmethyl)-benzonitrile (8.85 g, 33.7 mmol, 1 eq) in MeOH (150 mL) at 0° C. The reaction mixture was capped, allowed to warm gradually to room temperature and stir for 12 hours. The reaction mixture was then poured into ethyl ether to give a precipitate. The resulting solid precipitate was filtered, and dried under high vacuum to give 4-(1,3-dioxo-1,3-dihydro-isoindol-2-ylmethyl)-benzimidic acid methyl ester hydrochloride as a hygroscopic white powder (9.36 g, 84%).

Step 3

To a heterogeneous mixture of 4-(1,3-dioxo-1,3-dihydro-isoindol-2-ylmethyl)-benzimidic acid methyl ester hydrochloride (5.5 g, 16.6 mmol, 1 eq) and 2,3-diaminopropanoic acid monohydrochloride (4.0 g, 28.5 mmol, 1.7 q) in MeOH (100 mL) was carefully added triethylamine until pH is 9. The reaction mixture was heated under reflux for 1 hour, cooled and acidified carefully with conc. HCl to pH 1. The acidified mixture was heated under reflux for 12 hours, cooled and concentrated under reduced pressure. 2-[4-(1,3-Dioxo-1,3-dihydro-isoindol-2-ylmethyl)-phenyl]-4,5-dihydro-1H-imidazole-4-carboxylic acid was thus obtained as a white solid (8.0 g) and used for the next step without further purification. To a mixture of 2-[4-(1,3-dioxo-1,3-dihydro-isoindol-2-ylmethyl)-phenyl]-4,5-dihydro-1H-imidazole-4-carboxylic acid (8.0 g crude) and BrCCl₃ (8.0 g, 40.3 mmol, 2.4 eq) in acetonitrile (30 mL) was added DBU (8.0 g, 52.5 mmol, 3.2 eq). The initially exothermic reaction was allowed to cool, and stir for 12 hours. The reaction mixture was purified by silica gel column chromatography (acetonitrile) to give the desired product 2-[4-(1,3-dioxo-1,3-dihydro-isoindol-2-ylmethyl)-phenyl]-1H-imidazole-4-carboxylic acid methyl ester (2.0 g, 33%) as a white powder.

Step 4

To a solution of 2-[4-(1,3-dioxo-1,3-dihydro-isoindol-2-ylmethyl)-phenyl]-1H-imidazole-4-carboxylic acid methyl ester (0.35 g, 1 mmol, 1 eq) in ethanol (20 mL) was added hydrazine hydrate (0.032 g, 1 mmol, 1 eq) at room temperature. The reaction mixture was allowed to stir for 12 hours, and concentrated under reduced pressure. The mixture was column chromatographed (CH₂Cl₂) to give 2-(4-aminomethyl-phenyl)-1H-imidazole-4-carboxylic acid methyl ester (0.1 g, 43%) as a white powder.

Step 5

2-(4-Aminomethyl-phenyl)-1H-imidazole-4-carboxylic acid methyl ester was further treated as in Steps 5-7 of Example 1 to give N-{4-[5-(3,3-difluoro-piperidin-1-ylcarbonyl)-1H-imidazol-2-yl]-benzyl}-2-phenyl-acetamide. ¹H NMR (d₆-DMSO) δ 1.79 (2H), 2.28 (m, 2H), 3.5 (s, 2H), 3.73 (m, 1H), 4.05 (br s, 2H), 4.35 (d, 2H, J=6 Hz), 4.5 (br s, 1H), 7.25 (m, 1H), 7.31-7.29 (m, 4H), 7.38 (d, 2H, J=8 Hz), 7.58 (m, 1H), 7.97 (d, 2H., J=8 Hz), 8.67 (t, 1H, J=6 Hz); MS (ES) m/z 437.0 (M); MS calcd: 438.2 (M).

### Example 19

### Synthesis of 2-phenyl-N-{4-[5-(piperidin-1-ylcarbonyl)-[1,2,4]oxadiazol-3-yl]-benzyl}-acetamide

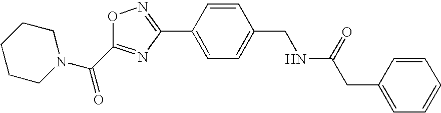

Step 1

A solution of 4-bromomethylbenzonitrile (100 mg, 0.5 mmol) was stirred in MeOH (1 mL) and conc. aqueous ammonia (3 mL) at room temperature overnight. The solution was concentrated, and then taken up in acetonitrile/isopropanol (1:1 v/v, 6 mL) and reconcentrated 2 times to give 4-aminomethyl-benzonitrile (63 mg, 98%).

Step 2

To a solution of 4-aminomethylbenzonitrile (62.7 mg, 0.48 mol) in DMF (4 mL) was added phenylacetic acid (95.2 mg, 0.7 mmol, 1.5 eq.), bromotripyrrolidino-phosphonium hexafluorophosphate (PyBrop) (296 mg, 0.7 mmol, 1.5 eq), and DIPEA (0.174 mL, 1.0 mmol, 2 eq) at room temperature. After 2 hours, the reaction mixture was partitioned between ethyl acetate (30 mL) and 1 N HCl (20 mL). The organic layer was washed subsequently with water (20 mL), saturated NaHCO₃ (20 mL), and brine (20 mL). The extract was dried (Na₂SO₄), filtered, and concentrated to give N-(4-cyanobenzyl)-2-phenylacetamide (121 mg, quantitative) as a white crystalline solid.

Step 3

To a solution of N-(4-cyanobenzyl)-2-phenylacetamide (121 mg, 0.48 mmol) in absolute ethanol (10 mL) was added aqueous hydroxylamine (50% aqueous solution, 0.10 mL). The reaction mixture was heated at reflux for 1.5 hours, at which time LC showed complete conversion. The solution was allowed to cool to room temperature and concentrated to give the desired product N-[4-(N-hydroxycarbamimidoyl)-benzyl]-2-phenylacetamide (89.6 mg, 66%) as a glass, which crystallized on standing.

Step 4

To a solution of N-[4-(N-hydroxycarbamimidoyl)-benzyl]-2-phenylacetamide (66 mg, 0.23 mmol) in DMF (5 mL) was added chlorooxoacetic acid methyl ester (0.32 mL, 0.35 mmol), and DIPEA (0.12 mL, 0.69 mmol). After one hour, LC showed no starting mterial remaining. The reaction mixture was taken up in EtOAc (20 mL) and washed with water (20 mL), and brine (20 mL). The extract was dried (Na₂SO₄), filtered and concentrated to give a mixture of oxalyl adduct and the desired product oxadiazole. The residue was taken up in THF (3 mL) and tetrabutylammonium fluoride (1 N in THF, 0.1 mL, 0.1 mmol) was added. After 45 min, LC showed complete conversion to the oxadiazole. The mixture was taken up in EtOAc (20 mL) and washed with water (20 mL), and brine (20 mL). The extract was dried (Na₂SO₄), filtered, and concentrated to give 3-[4-(phenylacetylaminomethyl)-phenyl]-[1,2,4]oxadiazole-5-carboxylic acid methyl ester (36 mg, 44%).

Step 5

A soluton of 3-[4-(phenylacetylaminomethyl)-phenyl]-[1,2,4]oxadiazole-5-carboxylic acid methyl ester (36 mg, 0.102 mmol) was heated at 50° C. in piperidine (0.06 mL, 0.6 mmol, 6 eq) for 30 min. The reaction mixture was taken up in EtOAc (30 mL) and washed successively with 1 N HCl (20 mL), water (20 mL), NaHCO₃ (20 mL), and brine (20 mL). The organic layer was dried (Na₂SO₄), filtered, and concentrated. The crude product was purified by flash column chromatography to give the desired product 2-phenyl-N-{4-[5-(piperidin-1-ylcarbonyl)-[1,2,4]oxadiazol-3-yl]-benzyl}-acetamide (28.2 mg, 70%). ¹H NMR (400 MHz, d₆-DMSO) δ 1.50-1.66 (m, 6H), 3.49 (s, 2H), 3.54-3.61 (m, 4H), 4.31 (d, 2H, J=5.9 Hz), 7.20-7.33 (m, 5H), 7.35 (d, 2H, J=8.6 Hz), 7.81 (d, 2H, J=8.6 Hz), 7.91 (s, 1H), 8.61 (t, 1H, J=5.9 Hz), 8.73 (s, 1H);MS (ES) m/z 405.1 (MH⁺); MS calcd: 404.1 (M).

### Biological Examples

### Example 1

### Identification of Caspase Cascade Activators and Inducers of Apoptosis in Solid Tumor Cells

Human breast cancer cell lines T-47D and ZR-75-1 were grown according to media component mixtures designated by American Type Culture Collection+10% fetal calf sera (FCS) (Invitrogen Corporation) in a 5% CO₂-95% humidity incubator as 37° C. The T-47 and ZR-75-1 cells were maintained at a cell density between 30 and 80% confluency at a cell density of 0.1 to 0.6×10⁶ cells/mL.

Cells were harvested at 6009 and resuspended at 0.65×10⁶ cells/mL into appropriate media+10% FCS. An aliquot of 45 μL of cells was added to a well of a 96-well microtiter plate containing 5 μL of a 10% DMSO in RPMI-1640 media solution containing 1.6 to 100/M of test compound (0.16 to 10 μM final). An aliquot of 45 μL of cells was added to a well of a 96-well microtiter plate containing 5 μL of a 10% DMSO in RPMI-1640 media solution without test compound as the control sample. The samples were mixed by agitation and then incubated at 37° C. for 24 hours in a 5% CO₂-95% humidity incubator. After incubation, the samples were removed from the incubator and 50 μL of a solution containing 20 ILL of N-(Ac-DEVD)-N′-ethoxycarbonyl-R¹¹⁰ fluorogenic substrate (Cytovia, Inc.; WO99/18856), 20% sucrose (Sigma), 20 mM dithiothreitol (DTT) (Sigma), 200 mM NaCl (Sigma), 40 mM Na piperazine-N,N′-bis[2-ethanesulfonic acid] (PIPES) buffer pH 7.2 (Sigma), and 500 μg/mL lysolecithin (Calbiochem) was added. The samples were mixed by agitation and incubated at room temperature. Using a fluorescent plate reader (Model 1420 Wallac Instruments), an initial reading (T=0) was made approximately 1-2 minutes after addition of the substrate solution, employing excitation at 485 nm and emission at 530 nm, to determine the background fluorescence of the control sample. After the 3 hour incubation, the samples were read for fluorescence as above (T=3 hours).

Calculation:

The Relative Fluorescence Unit (RFU) values were used to calculate the sample readings as follows:
RFU_{(T=3h)}−Control RFU_{(T=0)}=Net RFU_{(T=3h)}

The level of caspase cascade activation was determined by the ratio of the net RFU value for the test compound to that of the control samples. The EC₅₀ (nM) was determined by a sigmoidal dose-response calculation (Prism 2.0, GraphPad Software, Inc.). The compounds of the invention were determined to have caspase cascade activating effects by proceeding as in Example 1. The compounds of the present invention had an EC₅₀ value of less than 10 micromolar in T47D or ZR-75-1 cells.

### Example 2

### Identification of Antineoplastic Activity in Cell Proliferation

T-47D and ZR-75-1 cells are grown and harvested by proceeding as in Example 1.

An aliquot of 90 μL of cells (2.2×10⁴ cells/mL) is added to a well of a 96-well microtiter plate containing 10/L of a 10% DMSO in PRMI-1640 media solution containing 1 mM to 100 μM of test compound. An aliquot of 90 μL of cells is added to a well of a 96-well microtiter plate containing 10 μL of a 10% DMSO in RPMI-1640 media solution without test compound as the control sample for maximal cell proliferation (Amax). The samples are mixed by agitation and then incubated at 37° C. for 48 hours in a 5% CO₂-95% humidity incubator. After incubation, the samples are removed from the incubator and 20 μL of CellTiter 96 Aqueous One Solution Cell Proliferation®reagent (Promega) is added. The samples are mixed by agitation and incubated at 37° C. for 2-4 hours in a 5% CO₂-95% humidity incubator. Using an absorbance plate reader (Model 1420 Wallac Instruments), an initial reading (T=0) is made approximately 1-2 minutes after addition of the solution, employing absorbance at 490 nm, to determine any background absorbance of the test compound. After the 2-4 hours incubation, the samples are read for absorbance as above (Aₜₑₛₜ).

Baseline for the dose producing 50% inhibition of cell proliferation (GI₅₀) of initial cell numbers is determined by adding an aliquot of 90 μL of cells or 90 μL of media, respectively, to wells of a 96-well microtiter plate containing 10 μL of a 10% DMSO in RPMI-1640 media solution. The samples are mixed by agitation and then incubated at 37° C. for 0.5 hours in a 5% CO₂-95% humidity incubator. After incubation, the samples are removed from the incubator and 20 μL of CellTiter 96 Aqueous One Solution Cell Proliferation® reagent (Promega) is added. The samples are mixed by agitation and incubated at 37° C. for 2-4 hours in a 5% CO₂-95% humidity incubator. Absorbance is read as above, (AT=₀) defining absorbance for initial cell number used as baseline GI₅₀ determinations.

Calculation:
GI₅₀(nM)=100×[Aₜₑₛₜ−A_{T=0}/(Aₘₐₓ−A_{T-0})].

### Example 3

### Nuclear Fragmentation in T47D Cells

T47D cells are grown and harvested by proceeding as in Example 1 and treated with test compound followed by staining of the cell nuclei with Syto 16, a fluorescent DNA dye which stains nuclei. Shrunken and fragmented nuclei are hallmarks of caspase-mediated apoptosis. T47D cells treated with test compound for 48 hours exhibit shrunken and fragmented nuclei.

### Example 4

### Mitotic Arrest in Jurkat Cells

Jurkat cells are incubated with a range of concentrations of test compounds (0.02 μM to 5 μM) for 6 hours under normal growth conditions. Control cultures are treated with DMSO vehicle. The cells are then treated for 20 minutes with 800 nM Syto 16. Cytospin preparation is then prepared and the samples were viewed by fluorescent microscopy using a fluorescein filter set. For each concentration of test compound, the number of mitotic figures are counted and expressed as a percentage of the total number of cells. Three fields from each condition are evaluated and the mean and SEM were calculated and plotted as a function of drug concentration.

### Example 5

### Cell Cycle Arrest in Solid Tumor Cell Lines

T47D cells are grown and harvested by proceeding as in Example 1. 10⁶ Cells are treated with test compound for 48 hours at 37° C. As a control, cells are also incubated with DMSO. Cells were harvested at 1200 rpm and washed twice with 5 mM EDTA/PBS. Cells are then resuspended in 300 μL of EDTA/PBS and 700 mL of 100% ethanol, vortexed and incubated at room temperature for 1 hour. Samples are spun down at 12000 rpm for 5 minutes and the supernatant is removed. A solution containing 100 μg/mL of propidium iodide and 1 mg/mL of RNAse A (fresh) is added to the samples and the samples are incubated for 1 hour at room temperature. Samples are then transferred to 12×75 mm polystyrene tubes and analyzed on a flow cytometer. All flow cytometry analyses are performed on FACScalibur (Becton Dickison) using Cell Quest analysis software.

### Pharmaceutical Composition Examples

The following are representative pharmaceutical formulations containing a compound of Formula I or II

### Tablet Formulation

The following ingredients are mixed intimately and pressed into single scored tablets.

| Ingredient | Quantity per tablet, mg |
|---|---|
| compound of this invention | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| Ingredient | Quantity per tablet, mg |
|---|---|
| compound of this invention | 200 |
| lactose, spray-dried | 148 |
| magnesium stearate | 2 |

### Suspension Formulation

The following ingredients are mixed to form a suspension for oral administration.

| Ingredient | Amount |
|---|---|
| compound of this invention | 1.0 g |
| fumaric acid | 0.5 g |
| sodium chloride | 2.0 g |
| methyl paraben | 0.15 g |
| propyl paraben | 0.05 g |
| granulated sugar | 25.5 g |
| sorbitol (70% solution) | 12.85 g |
| Veegum K (Vanderbilt Co.) | 1.0 g |
| flavoring | 0.035 mL |
| colorings | 0.5 mg |
| distilled water | q.s. to 100 mL |

### Injectable Formulation

The following ingredients are mixed to form an injectable formulation.

| Ingredient | Amount |
|---|---|
| compound of this invention | 1.2 g |
| sodium acetate buffer solution | 0.4 M 2.0 mL |
| HCl (1 N) or NaOH (1 N) | q.s. to suitable pH |
| water (distilled, sterile) | q.s.to 20 mL |

All of the above ingredients, except water, are combined and heated to 60-70.degree. C. with stirring. A sufficient quantity of water at 60.degree. C. is then added with vigorous stirring to emulsify the ingredients, and water then added q.s. to 100 g.

### Suppository Formulation

A suppository of total weight 2.5 g is prepared by mixing the compound of the invention with Witepsol.RTM. H-15 (triglycerides of saturated vegetable fatty acid; Riches-Nelson, Inc., New York), and has the following composition:

| Ingredient | Quantity per tablet, mg |
|---|---|
| compound of this invention | 500 |
| Witepsol ® H-15 | balance |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

1. A compound of Formula I: 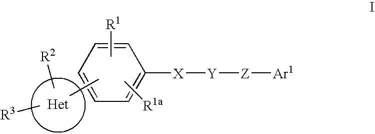
wherein:
R¹ and R^{1a} are independently hydrogen, alkyl, alkoxy, halo, haloalkyl, nitro, amino, alkylamino, dialkylamino, alkylcarbonylamino, carboxy, alkoxycarbonyl, carboxyalkyl, alkoxycarbonylalkenyl, hydroxy, hydroxyalkyl, alkoxycarbonylalkyloxy, alkoxycarbonylalkyl, carboxyalkylcarbonylamino, carboxyalkenyl, saturated or unsaturated heterocycloalkylaminocarbonylalkyl, or hydroxyalkyl; or when R¹ and R^{1a} are adjacent to each other they may combine to form a —CH═CH—CH═CH— group;
R² is hydrogen, alkyl, hydroxyalkyl, aryl, heteroaryl, or halo;
R is —CONR⁴R⁵ where R⁴ and R⁵ together with the nitrogen atom to which they are attached form saturated or unsaturated heterocycloalkylamino, saturated or unsaturated bicyclic heterocycloalkylamino, or saturated or unsaturated bridged heterocycloalkylamino;
Het is a five membered heteroaryl ring consisting of one, two, three, or four heteroatoms independently selected from nitrogen, oxygen, or sulfur, the remaining ring atoms being carbon;
X is alkylene optionally substituted with halo;
Y is —O—, —NR⁶—, —S—, —SO—, —SO₂—, —NR⁷CO—, —CONR⁷-, —NR⁷SO₂—, —SO₂NR⁷—, —NHCONH—, —NHCSNH—, —NHCOO—, or —OCONH— where R⁶ and R⁷ are independently hydrogen or alkyl;
Z is alkenylene or alkylene wherein said alkylene is optionally substituted with halo, hydroxy, hydroxyalkyl, carboxy, amino, amido, alkoxycarbonyl, alkylaminocarbonyl, or dialkylaminocarbonyl; and
Ar¹ is aryl, heteroaryl, or saturated or unsaturated heterocycloalkyl; or a pharmaceutically acceptable salt thereof, provided that: (i) when Het is oxazol-2-yl, R¹, R^{1a} and R² are hydrogen, X and Z are independently methylene, Y is —NHCO—, and Ar¹ is 4-methoxyphenyl, thien-2-yl, or 2,5-dimethoxyphenyl then R³ is not piperidin-1-yl, 4-methylpiperidin-1-yl, 4-phenylpiperazin-1-yl, 4-(2-methoxyphenyl)piperazin-1-yl, 4-methylpiperazin-1-yl, 4-acetylpiperazin-1-yl, or 3,4-methylenedioxybenzyl; and
(ii) when Het is oxazol-2-yl, R¹, R^{1a}, and R² are hydrogen, X is methylene, Y is —NHCO—, Z is ethylene, and Ar¹ is phenyl then R³ is not piperidin-1-yl, 4-methylpiperidin-1-yl, 4-phenylpiperazin-1-yl, 4-(2-methoxyphenyl)piperazin-1-yl, 4-methylpiperazin-1-yl, 4-acetylpiperazin-1-yl, or 3,4-methylenedioxybenzyl.

2. The compound of claim 1 wherein:
Het is oxazol-2-yl, thiazol-2-yl, 1H-imidazol-2-yl, [1,2,4]oxadiazol-3-yl, or 1H-pyrazol-1-yl and is located in the 4-position of the phenylene ring, with the carbon to which —X—Y—Z— is attached being in the 1-position;
R² is hydrogen, alkyl, or halo; and
Y is —NR⁷SO₂— or —NR⁷CO—.

3. The compound of claim 1 wherein:
Het is oxazol-2-yl and is located in the 4-position of the phenylene ring, with the carbon to which —X—Y—Z— is attached being in the 1-position;
R² is hydrogen, alkyl, or halo, and
Y is —NHCO—.

4. The compound of claim 2 wherein and R¹ and R^{1a} are hydrogen.

5. The compound of claim 2 wherein and R¹ and R^{1a} are halo.

6. The compound of claim 3 wherein and R¹, R^{1a}, and R² are hydrogen.

7. The compound of claim 3 wherein and R¹ and R^{1a} are halo and R² is hydrogen.

8. The compound of claim 6 wherein X is methylene or ethylene; and Z is alkylene or alkylene which is optionally substituted with one or two hydrogen, halo, hydroxy, hydroxyalkyl, carboxy, amino, alkoxycarbonyl, alkylaminocarbonyl, or dialkylaminocarbonyl.

9. The compound of claim 6 wherein X is methylene and Z is methylene, fluoromethylene, or difluoromethylene.

10. The compound of claim 7 wherein X is methylene or ethylene; and
Z is alkylene or alkylene which is optionally substituted with one or two hydrogen, halo, hydroxy, hydroxyalkyl, carboxy, amino, alkoxycarbonyl, alkylaminocarbonyl, or dialkylaminocarbonyl. 11. The compound of claim 7 wherein X is methylene and Z is methylene, fluoromethylene, or difluoromethylene.

12. The compound of claim 9 wherein Ar¹ is phenyl optionally substituted with one or two or three subsitutents independently selected from alkyl, halo, alkoxy, methylenedioxy, azido, haloalkyl, hydroxy, amino, cyano, or haloalkoxy.

13. The compound of claim 9 wherein Ar¹ is heteroaryl.

14. The compound of claim 11 wherein Ar¹ is phenyl optionally substituted with one or two or three subsitutents independently selected from alkyl, halo, alkoxy, methylenedioxy, azido, haloalkyl, hydroxy, amino, cyano, or haloalkoxy

15. The compound of claim 11 wherein Ar¹ is heteroaryl.

16. The compound of claim 12 wherein R³ is CONR⁴R⁵ where R⁴ and R⁵ together with the nitrogen atom to which they are attached form saturated heterocycloalkylamino.

17. The compound of claim 16 wherein R³ is CONR⁴R⁵ where R⁴ and R⁵ together with the nitrogen atom to which they are attached form 3,3-difluoropiperidin-1-yl, piperidin-1-yl, 4-hydroxypiperidin-1-yl, 3-hydroxypiperidin-1-yl, homopiperidin-1-yl, 4-hydroxyhomo-piperidin-1-yl, or 3,3-difluoro-4-hydroxypiperidin-1-yl.

18. The compound of claim 13 wherein R³ is —CONR⁴R⁵ where R⁴ and R⁵ together with the nitrogen atom to which they are attached form saturated heterocycloalkylamino.

19. The compound of claim 18 wherein R³ is CONR⁴R⁵ where R⁴ and R⁵ together with the nitrogen atom to which they are attached form 3,3-difluoropiperidin-1-yl, piperidin-1-yl, 4-hydroxypiperidin-1-yl, 3-hydroxypiperidin-1-yl, homopiperidin-1-yl, 4-hydroxyhomo-piperidin-1-yl, or 3,3-difluoro-4-hydroxypiperidin-1-yl.

20. The compound of claim 14 wherein R³ is CONR⁴R⁵ where R⁴ and R⁵ together with the nitrogen atom to which they are attached form saturated heterocycloalkylamino.

21. The compound of claim 20 wherein R³ is CONR⁴R⁵ where R⁴ and R⁵ together with the nitrogen atom to which they are attached form 3,3-difluoropiperidin-1-yl, piperidin-1-yl, 4-hydroxypiperidin-1-yl, 3-hydroxypiperidin-1-yl, homopiperidin-1-yl, 4-hydroxyhomo-piperidin-1-yl, or 3,3-difluoro-4-hydroxypiperidin-1-yl.

22. The compound of claim 15 wherein R³ is CONR⁴R⁵ where R⁴ and R⁵ together with the nitrogen atom to which they are attached form saturated heterocycloalkylamino.

23. The compound of claim 22 wherein R³ is —CONR⁴R⁵ where R⁴ and R⁵ together with the nitrogen atom to which they are attached form 3,3-difluoropiperidin-1-yl, piperidin-1-yl, 4-hydroxypiperidin-1-yl, 3-hydroxypiperidin-1-yl, homopiperidin-1-yl, 4-hydroxyhomo-piperidin-1-yl, or 3,3-difluoro-4-hydroxypiperidin-1-yl.

24. The compound of claim 1 wherein:
Het is oxazol-2-yl, thiazol-2-yl, 1H-imidazol-2-yl, [1,2,4]oxadiazol-3-yl, or 1H-pyrazol-1-yl and is located in the 4-position of the phenylene ring, with the carbon to which —X—Y—Z— is attached being in the 1-position;
R² is hydrogen, alkyl, or halo;
Y is —NR⁷CO—;
X is alkylene; and
Z is alkylene or alkylene optionally substituted with one or two halo.

25. The compound of claim 1 wherein:
Het is oxazol-2-yl, thiazol-2-yl, 1H-imidazol-2-yl, [1,2,4]oxadiazol-3-yl, or 1H-pyrazol-1-yl and is located in the 4-position of the phenylene ring, with the carbon to which —X—Y—Z— is attached being in the 1-position;
R² is hydrogen, alkyl, or halo;
Y is —NR⁷SO₂— or —NR⁷CO—;
X is methylene; and
Z is methylene or difluoromethylene.

26. A compound selected from the group consisting of:
2-phenyl-N-{4-[4-(3,3-difluoropiperidin-1-ylcarbonyl)-oxazol-2-yl]-benzyl}-acetamide;
2-(4-trifluoromethoxyphenyl)-N-{4-[4-(piperidin-1-ylcarbonyl)-oxazol-2-yl]-benzyl}-acetamide;
2-fluoro-2-phenyl-N-{4-[4-(4-hydroxypiperidin-1-ylcarbonyl)-oxazol-2-yl]-benzyl}-acetamide;
2-phenyl-N-{4-[4-(3-hydroxypiperidin-1-ylcarbonyl)-oxazol-2-yl]-benzyl}-acetamide;
2-(4-chlorophenyl)-N-{4-[4-(piperidin-1-ylcarbonyl)-oxazol-2-yl]-benzyl}-acetamide;
2-(thien-3-yl)-N-{4-[4-(3-hydroxypiperidin-1-ylcarbonyl)-oxazol-2-yl]-benzyl}-acetamide;
2-(thien-3-yl)-N-{4-[4-(3,3-difluoropiperidin-1-ylcarbonyl)-oxazol-2-yl]-benzyl}-acetamide;
2-(thien-3-yl)-N-{4-[4-(homopiperidin-1-ylcarbonyl)-oxazol-2-yl]-benzyl}-acetamide;
2-phenyl-N-{4-[4-(piperidin-1-ylcarbonyl)-oxazol-2-yl]-benzyl}-acetamide;
2-fluoro-2-phenyl-N-{4-[4-(piperidin-1-ylcarbonyl)-oxazol-2-yl]-benzyl}-acetamide;
2-(thien-3-yl)-N-{4-[4-(piperidin-1-ylcarbonyl)-oxazol-2-yl]-benzyl}-acetamide;
2,2-difluoro-2-(thien-3-yl)-N-{4-[4-(piperidin-1-ylcarbonyl)-oxazol-2-yl]-benzyl}-acetamide;
2-phenyl-N-{4-[4-(homopiperidin-1-ylcarbonyl)-oxazol-2-yl]-benzyl}-acetamide;
2,2-difluoro-2-(thien-2-yl)-N-{4-[4-(4-hydroxyhomopiperidin-1-ylcarbonyl)-oxazol-2-yl]-benzyl}-acetamide;
2,2-difluoro-2-(thien-2-yl)-N-{4-[4-(3-hydroxypiperidin-1-ylcarbonyl)-oxazol-2-yl]-benzyl}-acetamide;
2-(thien-3-yl)-N-{4-[4-(4-hydroxyhomopiperidin-1-ylcarbonyl)-oxazol-2-yl]-benzyl}-acetamide; 2-(thien-3-yl)-N-{4-[4-(3,3-difluoro-4-hydroxypiperidin-1-ylcarbonyl)-oxazol-2-yl]-benzyl}-acetamide;
2-fluoro-2-(2-fluorophenyl)-N-{4-[4-(piperidin-1-ylcarbonyl)-oxazol-2-yl]-benzyl}-acetamide;
3-(2-methoxyphenyl)-N-{4-[4-(piperidin-1-ylcarbonyl)-oxazol-2-yl]-benzyl}-propionamide;
3-(3-methoxyphenyl)-N-{4-[4-(piperidin-1-ylcarbonyl)-oxazol-2-yl]-benzyl}-propionamide;
3-(4-methoxyphenyl)-N-{4-[4-(piperidin-1-ylcarbonyl)-oxazol-2-yl]-benzyl}-propionamide;
3-(4-methylphenyl)-N-{4-[4-(piperidin-1-ylcarbonyl)-oxazol-2-yl]-benzyl}-propionamide;
3-(3,4-difluorophenyl)-N-{4-[4-(piperidin-1-ylcarbonyl)-oxazol-2-yl]-benzyl}-propionamide;
3-[2,5-bis-(trifluoromethyl)phenyl]-N-{4-[4-(piperidin-1-ylcarbonyl)-oxazol-2-yl]-benzyl}-propionamide;
3-(3-fluorophenyl)-N-{4-[4-(piperidin-1-ylcarbonyl)-oxazol-2-yl]-benzyl}-propionamide;
3-(2-fluorophenyl)-N-{4-[4-(piperidin-1-ylcarbonyl)-oxazol-2-yl]-benzyl}-propionamide;
3-(3,4-methylenedioxyphenyl)-N-{4-[4-(piperidin-1-ylcarbonyl)-oxazol-2-yl]-benzyl}-propionamide;
3-(3,4-dichlorophenyl)-N-{4-[4-(piperidin-1-ylcarbonyl)-oxazol-2-yl]-benzyl}-propionamide;
3-(2,6-dichlorophenyl)-N-{4-[4-(piperidin-1-ylcarbonyl)-oxazol-2-yl]-benzyl}-propionamide;
3-(3-methylphenyl)-N-{4-[4-(piperidin-1-ylcarbonyl)-oxazol-2-yl]-benzyl}-propionamide;
3-(4-fluorophenyl)-N-{4-[4-(piperidin-1-ylcarbonyl)-oxazol-2-yl]-benzyl}-propionamide;
3-(2,4-dichlorophenyl)-N-{4-[4-(piperidin-1-ylcarbonyl)-oxazol-2-yl]-benzyl}-propionamide, 3-(2,5-dimethoxyphenyl)-N-{4-[4-(piperidin-1-ylcarbonyl)-oxazol-2-yl]-benzyl}-propionamide;
2-methyl-3-(phenyl)-N-{4-[4-(piperidin-1-ylcarbonyl)-oxazol-2-yl]-benzyl}-propionamide;
3-methyl-3-(phenyl)-N-{4-[4-(piperidin-1-ylcarbonyl)-oxazol-2-yl]-benzyl}-propionamide;
3-(2-methylphenyl)-N-{4-[4-(piperidin-1-ylcarbonyl)-oxazol-2-yl]-benzyl}-propionamide;
2-(3,4-methylenedioxyphenyl)-N-{4-[4-(piperidin-1-ylcarbonyl)-oxazol-2-yl]-benzyl}-acetamide;
2-(4-methoxy-3-methylphenyl)-N-{4-[4-(piperidin-1-ylcarbonyl)-oxazol-2-yl]-benzyl}-acetamide;
2-(3,4,5-trimethoxyphenyl)-N-{4-[4-(piperidin-1-ylcarbonyl)-oxazol-2-yl]-benzyl}-acetamide;
2-(4-methylphenyl)-N-{4-[4-(piperidin-1-ylcarbonyl)-oxazol-2-yl]-benzyl}-acetamide;
2-(pyridin-2-yl)-N-{4-[4-(piperidin-1-ylcarbonyl)-oxazol-2-yl]-benzyl}-acetamide;
2-(3,4-dimethoxyphenyl)-N-{4-[4-(piperidin-1-ylcarbonyl)-oxazol-2-yl]-benzyl}-acetamide;
3-(pyridin-2-yl)-N-{4-[4-(piperidin-1-ylcarbonyl)-oxazol-2-yl]-benzyl}-propionamide; 2-(4-methoxyphenyl)-N-{4-[4-(2-methylpiperidin-1-ylcarbonyl)-oxazol-2-yl]-benzyl}-acetamide;
3-(phenyl)-N-{4-[4-(2-methylpiperidin-1-ylcarbonyl)-oxazol-2-yl]-benzyl}-propionamide;
2-(4-ethoxyphenyl)-N-{4-[4-(piperidin-1-ylcarbonyl)-oxazol-2-yl]-benzyl}-acetamide;
2-(4-methoxyphenyl)-N-{4-[4-(3-methylpiperidin-1-ylcarbonyl)-oxazol-2-yl]-benzyl}-acetamide;
3-(furan-2-yl)-N-{4-[4-(piperidin-1-ylcarbonyl)-oxazol-2-yl]-benzyl}-propionamide;
3-(phenyl)-N-{4-[4-(3-methylpiperidin-1-ylcarbonyl)-oxazol-2-yl]-benzyl}-propionamide;
4-(thien-2-yl)-N-{4-[4-(piperidin-1-ylcarbonyl)-oxazol-2-yl]-benzyl}-butyramide;
2-(pyridin-3-yl)-N-{4-[4-(piperidin-1-ylcarbonyl)-oxazol-2-yl]-benzyl}-acetamide;
2-(3,5-dimethylphenyl)-N-{4-[4-(piperidin-1-ylcarbonyl)-oxazol-2-yl]-benzyl}-acetamide;
3-(thien-2-yl)-N-{4-[4-(piperidin-1-ylcarbonyl)-oxazol-2-yl]-benzyl}-propionamide;
2-(thien-2-yl)-N-{4-[4-(3-methylpiperidin-1-ylcarbonyl)-oxazol-2-yl]-benzyl}-acetamide;
2-(thien-2-yl)-N-{4-[4-(thiomorpholin-4-ylcarbonyl)-oxazol-2-yl]-benzyl}-acetamide;
2-(thien-2-yl)-N-{4-[4-(1,4-dioxa-8-aza-spiro[4.5]decan-8-ylcarbonyl)-oxazol-2-yl]-benzyl}-acetamide;
2-(thien-2-yl)-N-{4-[4-(2,6-dimethylpiperidin-1-ylcarbonyl)-oxazol-2-yl]-benzyl}-acetamide;
2-(thien-2-yl)-N-{4-[4-(2-methylpiperidin-1-ylcarbonyl)-oxazol-2-yl]-benzyl}-acetamide;
3-(phenyl)-N-{4-[4-(3,5-dimethylpiperidin-1-ylcarbonyl)-oxazol-2-yl]-benzyl}-propionamide;
3-(phenyl)-N-{4-[4-(4-hydroxymethylpiperidin-1-ylcarbonyl)-oxazol-2-yl]-benzyl}-propionamide;
2-(4-methoxyphenyl)-N-{4-[4-(4-hydroxypiperidin-1-ylcarbonyl)-oxazol-2-yl]-benzyl}-acetamide;
2,2-dimethyl-N-methyl-2-phenyl-N-{4-[4-(piperidin-1-ylcarbonyl)-oxazol-2-yl]-benzyl}-acetamide;
N-methyl-2-phenyl-N-{4-[4-(piperidin-1-ylcarbonyl)-oxazol-2-yl]-benzyl}-acetamide;
2-(4-methoxyphenyl)-N-{4-[4-(thiomorpholin-4-ylcarbonyl)-oxazol-2-yl]-benzyl}-acetamide;
2-(4-methoxyphenyl)-N-{4-[4-(1,2,3,6-tetrahydro-pyridin-1-ylcarbonyl)-oxazol-2-yl]-benzyl}-acetamide;
2,2-difluoro-2-phenyl-N-{4-[4-(piperidin-1-ylcarbonyl)-oxazol-2-yl]-benzyl}-acetamide;
2-(thien-2-yl)-N-{4-[4-(pyrrolidin-1-ylcarbonyl)-oxazol-2-yl]-benzyl}-acetamide;
2-(thien-2-yl)-N-{4-[4-(4-bromopiperidin-1-ylcarbonyl)-oxazol-2-yl]-benzyl}-acetamide;
2-(thien-2-yl)-N-{4-[4-(1,2,3,6-tetrahydro-pyridin-1-ylcarbonyl)-oxazol-2-yl]-benzyl}-acetamide;
2-(4-methoxyphenyl)-N-{4-[4-(homopiperidin-1-ylcarbonyl)-oxazol-2-yl]-benzyl}-acetamide;
2-(4-methoxyphenyl)-N-{4-[4-(4-hydroxymethylpiperidin-1′-ylcarbonyl)-oxazol-2-yl]-benzyl}-acetamide;
3-phenyl-N-{4-[4-(2,6-dimethylpiperidin-1-ylcarbonyl)-oxazol-2-yl]-benzyl}-propionamide; 2-phenyl-N-{4-[5-methyl-4-(piperidin-1-ylcarbonyl)-oxazol-2-yl]-benzyl}-acetamide;
2-(thien-2-yl)-N-{4-[5-methyl-4-(piperidin-1-ylcarbonyl)-oxazol-2-yl]-benzyl}-acetamide;
2-(4-methoxyphenyl)-N-{4-[4-(2,6-dimethylpiperidin-1-ylcarbonyl)-oxazol-2-yl]-benzyl}-acetamide;
2-phenyl-N-{4-[4-(4-methylpiperidin-1-ylcarbonyl)-oxazol-2-yl]-benzyl}-acetamide;
2-phenyl-N-{4-[4-(2-methylpiperidin-1-ylcarbonyl)-oxazol-2-yl]-benzyl}-acetamide;
2-phenyl-N-{4-[4-(4-hydroxypiperidin-1-ylcarbonyl)-oxazol-2-yl]-benzyl}-acetamide;
2-phenyl-N-{4-[4-(thiomorpholin-4-ylcarbonyl)-oxazol-2-yl]-benzyl}-acetamide;
2-phenyl-N-{4-[4-(3-methylpiperidin-1-ylcarbonyl)-oxazol-2-yl]-benzyl}-acetamide;
2-phenyl-N-{4-[4-(morpholin-4-ylcarbonyl)-oxazol-2-yl]-benzyl}-acetamide;
2-phenyl-N-{4-[4-(4-hydroxypiperidin-1-ylcarbonyl)-oxazol-2-yl]-benzyl}-acetamide;
2-phenyl-N-{4-[4-(4-bromopiperidin-1-ylcarbonyl)-oxazol-2-yl]-benzyl}-acetamide;
2-phenyl-N-{4-[4-(1,2,3,6-tetrahydro-pyridin-1-ylcarbonyl)-oxazol-2-yl]-benzyl}-acetamide;
2-phenyl-N-{4-[4-(pyrrolidin-1-ylcarbonyl)-oxazol-2-yl]-benzyl}-acetamide;
2-phenyl-N-{4-[5-methyl-4-(2-methylpiperidin-1-ylcarbonyl)-oxazol-2-yl]-benzyl}-acetamide;
2-(thien-2-yl)-N-{4-[5-methyl-4-(2-methylpiperidin-1-ylcarbonyl)-oxazol-2-yl]-benzyl}-acetamide;
2-(4-methoxyphenyl)-N-{4-[4-(2,6-dimethylpiperidin-1-ylcarbonyl)-oxazol-2-yl]-benzyl}-acetamide;
2,2-difluoro-2-phenyl-N-{4-[4-(homopiperidin-1-ylcarbonyl)-oxazol-2-yl]-benzyl}-acetamide;
2,2-difluoro-2-phenyl-N-{4-[4-(1,2,3,6-tetrahydro-pyridin-1-ylcarbonyl)-oxazol-2-yl]-benzyl}-acetamide;
2-fluoro-2-phenyl-N-{4-[4-(homopiperidin-1-ylcarbonyl)-oxazol-2-yl]-benzyl}-acetamide;
2-fluoro-2-phenyl-N-{4-L4-(1,2,3,6-tetrahydro-pyridin-1-ylcarbonyl)-oxazol-2-yl]-benzyl}-acetamide;
2-fluoro-2-phenyl-N-{4-[4-(pyrrolidin-1-ylcarbonyl)-oxazol-2-yl]-benzyl}-acetamide;
2-(2-fluorophenyl)-N-{4-[4-(piperidin-1-ylcarbonyl)-oxazol-2-yl]-benzyl}-acetamide;
2-(3-fluorophenyl)-N-{4-[4-(piperidin-1-ylcarbonyl)-oxazol-2-yl]-benzyl}-acetamide;
2-(4-fluorophenyl)-N-{4-[4-(piperidin-1-ylcarbonyl)-oxazol-2-yl]-benzyl}-acetamide;
2-(2,6-difluorophenyl)-N-{4-[4-(piperidin-1-ylcarbonyl)-oxazol-2-yl]-benzyl}-acetamide;
2-(3-chlorophenyl)-N-{4-[4-(piperidin-1-ylcarbonyl)-oxazol-2-yl]-benzyl}-acetamide;
2-phenyl-N-{4-[4-(piperidin-1-ylcarbonyl)-oxazol-2-yl]-phenethyl}-acetamide;
2-(furan-2-yl)-N-{4-[4-(piperidin-1-ylcarbonyl)-oxazol-2-yl]-benzyl}-acetamide;
2-(4-trifluoromethoxyphenyl)-N-{4-[4-(3-hydroxypiperidin-1-ylcarbonyl)-oxazol-2-yl]-benzyl}-acetamide;
2-(4-trifluoromethoxyphenyl)-N-{4-[4-(4-hydroxypiperidin-1-ylcarbonyl)-oxazol-2-yl]-benzyl}-acetamide;
2-phenyl-N-{4-[5-bromo-4-(piperidin-1-ylcarbonyl)-oxazol-2-yl]-benzyl}-acetamide;
2-phenyl-N-{4-[4-(azetidin-1-ylcarbonyl)-oxazol-2-yl]-benzyl}-acetamide;
2-phenyl-N-{4-[4-(2-methylpyrrolidin-1-ylcarbonyl)-oxazol-2-yl]-benzyl}-acetamide;
2-phenyl-N-{4-[4-(3-hydroxypyrrolidin-1-ylcarbonyl)-oxazol-2-yl]-benzyl}-acetamide;
2-phenyl-N-{4-[4-(2,5-dimethylpyrrolidin-1-ylcarbonyl)-oxazol-2-yl]-benzyl}-acetamide;
2-phenyl-N-{4-[4-(trans-2,5-dimethyl-2,5-dihydro-1H-pyrrol-1-ylcarbonyl)-oxazol-2-yl]-benzyl}-acetamide;
2-phenyl-N-{4-[4-(thiazolidin-3-ylcarbonyl)-oxazol-2-yl]-benzyl}-acetamide;
2-phenyl-N-{4-[4-(2-methylthiazolidin-3-ylcarbonyl)-oxazol-2-yl]-benzyl}-acetamide;
2-phenyl-N-{4-[4-(3,3-dimethylpiperidin-1-ylcarbonyl)-oxazol-2-yl]-benzyl}-acetamide;
2-phenyl-N-{4-[4-(piperazin-1-ylcarbonyl)-oxazol-2-yl]-benzyl}-acetamide;
2-phenyl-N-{4-[4-(4-acetylpiperazin-1-ylcarbonyl)-oxazol-2-yl]-benzyl}-acetamide;
2-phenyl-N-{4-[4-(1-oxothiomorpholin-4-ylcarbonyl)-oxazol-2-yl]-benzyl}-acetamide;
2-phenyl-N-{4-[4-(1,1-dioxothiomorpholin-4-ylcarbonyl)-oxazol-2-yl]-benzyl}-acetamide;
N-methyl-2-phenyl-N-{4-[4-(3-hydroxypiperidin-1-ylcarbonyl)-oxazol-2-yl]-benzyl}-acetamide;
2-phenyl-N-{4-[4-(3-methoxypiperidin-1-ylcarbonyl)-oxazol-2-yl]-benzyl}-acetamide;
N-methyl-2-phenyl-N-{4-[4-(3-methoxypiperidin-1-yl carbonyl)-oxazol-2-yl]-benzyl}-acetamide;
N-methyl-2-phenyl-N-{4-[4-(4-hydroxypiperidin-1-ylcarbonyl)-oxazol-2-yl]-benzyl}-acetamide;
2-phenyl-N-{4-[4-(4-methoxypiperidin-1-ylcarbonyl)-oxazol-2-yl]-benzyl}-acetamide;
N-methyl-2-phenyl-N-{4-[4-(4-methoxypiperidin-1-ylcarbonyl)-oxazol-2-yl]-benzyl}-acetamide;
2-phenyl-N-{4-[4-(homopiperazin-1-ylcarbonyl)-oxazol-2-yl]-benzyl}-acetamide;
2-phenyl-N-{4-[4-(4-methylhomopiperazin-1-ylcarbonyl)-oxazol-2-yl]-benzyl}-acetamide;
2-phenyl-N-{4-[4-(azocan-1-ylcarbonyl)-oxazol-2-yl]-benzyl}-acetamide; 2-phenyl-N-{4-[4-(1,2,3,4-tetrahydro-isoquinolin-2-ylcarbonyl)-oxazol-2-yl]-benzyl}-acetamide;
2-phenyl-N-{4-[4-(decahydroisoquinolin-2-ylcarbonyl)-oxazol-2-yl]-benzyl}-acetamide;
2-phenyl-N-{4-[4-(3-aza-bicyclo[2.2.1]hept-5-en-3-ylcarbonyl)-oxazol-2-yl]-benzyl}-acetamide;
2-phenyl-N-{4-[4-(3-aza-bicyclo[3.2.2]non-6-ene-3-ylcarbonyl)-oxazol-2-yl]-benzyl}-acetamide;
2-phenyl-N-{4-[4-(4-fluoropiperidin-1-ylcarbonyl)-oxazol-2-yl]-benzyl}-acetamide;
2-phenyl-N-{4-[4-(4,4-difluoropiperidin-1-ylcarbonyl)-oxazol-2-yl]-benzyl}-acetamide;
2-phenyl-N-{4-[4-(2-methylaziridin-1-ylcarbonyl)-oxazol-2-yl]-benzyl}-acetamide;
2-(4-trifluoromethoxyphenyl)-N-{4-[4-(4-hydroxymethylpiperidin-1-ylcarbonyl)-oxazol-2-yl]-benzyl}-acetamide;
2-fluoro-2-phenyl-N-{4-[4-(3-hydroxypiperidin-1-ylcarbonyl)-oxazol-2-yl]-benzyl}-acetamide;
2-fluoro-2-phenyl-N-{4-[4-(4-hydroxypiperidin-1-ylcarbonyl)-oxazol-2-yl]-benzyl}-acetamide;
2-(thien-3-yl)-N-{4-[4-(4-hydroxypiperidin-1-ylcarbonyl)-oxazol-2-yl]-benzyl}-acetamide;
2-(furan-2-yl)-N-{4-[4-(4-hydroxypiperidin-1-ylcarbonyl)-oxazol-2-yl]-benzyl}-acetamide;
2-(furan-2-yl)-N-{4-[4-(3-hydroxypiperidin-1-ylcarbonyl)-oxazol-2-yl]-benzyl}-acetamide;
2-(furan-2-yl)-N-{4-[4-(4-hydroxymethylpiperidin-1-ylcarbonyl)-oxazol-2-yl]-benzyl}-acetamide;
2-phenyl-N-{4-[4-(3-fluoropiperidin-1-ylcarbonyl)-oxazol-2-yl]-benzyl}-acetamide;
2-phenyl-N-{4-[4-(4-oxopiperidin-1-ylcarbonyl)-oxazol-2-yl]-benzyl}-acetamide;
2-phenyl-N-{4-[4-(trans-2,5-dimethylpiperazin-1-ylcarbonyl)-oxazol-2-yl]-benzyl}-acetamide;
2-phenyl-N-{4-[4-(3-oxopiperidin-1-ylcarbonyl)-oxazol-2-yl]-benzyl}-acetamide;
2-phenyl-N-{4-[4-(4-chloropiperidin-1-ylcarbonyl)-oxazol-2-yl]-benzyl}-acetamide;
2-phenyl-N-{4-[4-(3-chloropiperidin-1-ylcarbonyl)-oxazol-2-yl]-benzyl}-acetamide;
2-fluoro-2-phenyl-N-{4-[4-(4-fluoropiperidin-1-ylcarbonyl)-oxazol-2-yl]-benzyl}-acetamide;
2-fluoro-2-phenyl-N-{4-[4-(4-methoxypiperidin-1-ylcarbonyl)-oxazol-2-yl]-benzyl}-acetamide;
2-fluoro-2-phenyl-N-{4-[4-(4-carboxypiperidin-1-ylcarbonyl)-oxazol-2-yl]-benzyl}-acetamide;
1-phenyl-N-{4-[4-(piperidin-1-ylcarbonyl)-oxazol-2-yl]-benzyl}-methanesulfonamide;
2-fluoro-2-phenyl-N-{4-[4-(4-ethoxypiperidin-1-ylcarbonyl)-oxazol-2-yl]-benzyl}-acetamide;
2-phenyl-N-{4-[4-(3-methoxypiperidin-1-ylcarbonyl)-oxazol-2-yl]-benzyl}-acetamide;
2-fluoro-2-phenyl-N-{4-[4-(3,3-difluoropiperidin-1-ylcarbonyl)-oxazol-2-yl]-benzyl}-acetamide;
2-fluoro-2-phenyl-N-{4-[4-(azocan-1-ylcarbonyl)-oxazol-2-yl]-benzyl}-acetamide;
2-fluoro-2-phenyl-N-{4-[4-(2-methylpyrrolidin-1-ylcarbonyl)-oxazol-2-yl]-benzyl}-acetamide;
2-(4-trifluoromethoxyphenyl)-N-{4-[4-(morpholin-4-ylcarbonyl)-oxazol-2-yl]-benzyl}-acetamide;
2-fluoro-2-phenyl-N-{4-[4-(morpholin-4-ylcarbonyl)-oxazol-2-yl]-benzyl}-acetamide;
2-(thien-3-yl)-N-{4-[4-(morpholin-4-ylcarbonyl)-oxazol-2-yl]-benzyl}-acetamide;
2-fluoro-2-phenyl-N-{4-[4-(2,5-dimethylpyrrolidin-1-ylcarbonyl)-oxazol-2-yl]-benzyl}-acetamide;
2-phenyl-N-{4-[4-(2S-methoxycarbonylpyrrolidin-1-ylcarbonyl)-oxazol-2-yl]-benzyl}-acetamide;
2-phenyl-N-{4-[4-(2S-hydroxymethylpyrrolidin-1-ylcarbonyl)-oxazol-2-yl]-benzyl}-acetamide;
2-phenyl-N-{4-[4-(2R-hydroxymethylpyrrolidin-1-ylcarbonyl)-oxazol-2-yl]-benzyl}-acetamide;
2-phenyl-N-{4-[4-(trans-2,5-dimethylpyrrolidin-1-ylcarbonyl)-oxazol-2-yl]-benzyl}-acetamide;
2-(thien-3-yl)-N-{4-[4-(2,5-dimethylpyrrolidin-1-ylcarbonyl)-oxazol-2-yl]-benzyl}-acetamide;
2-(thien-3-yl)-N-{4-[4-(1,2,3,6-tetrahydro-pyridin-1-ylcarbonyl)-oxazol-2-yl]-benzyl}-acetamide;
2-(thien-3-yl)-N-{4-[4-(3-methylpiperidin-1-ylcarbonyl)-oxazol-2-yl]-benzyl}-acetamide;
2-(thien-3-yl)-N-{4-[4-(2-methylpyrrolidin-1-ylcarbonyl)-oxazol-2-yl]-benzyl}-acetamide;
2-phenyl-N-{4-[4-(cis-3,4-dihydroxypiperidin-1-ylcarbonyl)-oxazol-2-yl]-benzyl}-acetamide;
2-(thien-3-yl)-N-{4-[4-(3-chloropiperidin-1-ylcarbonyl)-oxazol-2-yl]-benzyl}-acetamide;
2-(thien-3-yl)-N-{4-[4-(4-chloropiperidin-1-ylcarbonyl)-oxazol-2-yl]-benzyl}-acetamide;
2-(thien-3-yl)-N-{4-[4-(3,5-dimethylpiperidin-1-ylcarbonyl)-oxazol-2-yl]-benzyl}-acetamide;
2-(thien-3-yl)-N-{4-{4-[2-(2-hydroxyethyl)piperidin-1-ylcarbonyl]-oxazol-2-yl}-benzyl}-acetamide;
2-(thien-3-yl)-N-{4-[4-(2,6-dimethylpiperidin-1-ylcarbonyl)-oxazol-2-yl]-benzyl}-acetamide;
2-(thien-3-yl)-N-{4-[4-(4,4-difluoropiperidin-1-ylcarbonyl)-oxazol-2-yl]-benzyl}-acetamide;
2-(thien-3-yl)-N-{4-[4-(4-fluoropiperidin-1-ylcarbonyl)-oxazol-2-yl]-benzyl}-acetamide;
2-phenyl-N-{4-[4-(3,4-difluoropiperidin-1-ylcarbonyl)-oxazol-2-yl]-benzyl}-acetamide;
sulfuric acid mono-(3-hydroxy-1-{2-[4-(phenylacetylamino-methyl)-phenyl]-oxazol-4-ylcarbonyl}-piperidin-4-yl) ester;
2-(thien-3-yl)-N-{4-[4-(3-methoxypiperidin-1-ylcarbonyl)-oxazol-2-yl]-benzyl}-acetamide;
2-(thien-3-yl)-N-{4-[4-(3-hydroxypiperidin-1-ylcarbonyl)-oxazol-2-yl]-benzyl}-acetamide;
2-(thien-3-yl)-N-{4-[4-(2-hydroxymethylpiperidin-1-ylcarbonyl)-oxazol-2-yl]-benzyl}-acetamide;
2-(thien-3-yl)-N-{4-[4-(thiomorpholin-4-ylcarbonyl)-oxazol-2-yl]-benzyl}-acetamide;
2-(thien-3-yl)-N-{4-[4-(azocan-1-ylcarbonyl)-oxazol-2-yl]-benzyl}-acetamide;
2-(thien-3-yl)-N-{4-[4-(4-methylpiperidin-1-ylcarbonyl)-oxazol-2-yl]-benzyl}-acetamide;
2-(thien-3-yl)-N-{4-[4-(3-fluoropiperidin-1-ylcarbonyl)-oxazol-2-yl]-benzyl}-acetamide;
2-phenyl-N-{4-[4-(3-hydroxy-(homopiperidin-1-yl)carbonylcarbonyl)-oxazol-2-yl]-benzyl}-acetamide;
2-phenyl-N-{4-[4-(4-hydroxy-(homopiperidin-1-yl)carbonylcarbonyl)-oxazol-2-yl]-benzyl}-acetamide;
2-phenyl-N-{4-[4-(3R-hydroxypiperidin-1-ylcarbonyl)-oxazol-2-yl]-benzyl}-acetamide;
2-(thien-3-yl)-N-{4-[4-(3-hydroxy-(homopiperidin-1-yl)carbonylcarbonyl)-oxazol-2-yl]-benzyl}-acetamide;
2-(thien-3-yl)-N-{4-[4-(3R-hydroxypiperidin-1-ylcarbonyl)-oxazol-2-yl]-benzyl}-acetamide;
2,2-difluoro-2-(thien-3-yl)-N-{4-[4-(4-hydroxypiperidin-1-ylcarbonyl)-oxazol-2-yl]-benzyl}-acetamide;
2-(thien-3-yl)-N-{4-[4-(3-trifluoromethylpiperidin-1-ylcarbonyl)-oxazol-2-yl]-benzyl}-acetamide;
2-phenyl-N-{4-[4-(3-trifluoromethylpiperidin-1-ylcarbonyl)-oxazol-2-yl]-benzyl}-acetamide;
2,2-difluoro-2-(thien-3-yl)-N-{4-[4-(3,3-difluoropiperidin-1-ylcarbonyl)-oxazol-2-yl]-benzyl}-acetamide;
2,2-difluoro-2-(thien-2-yl)-N-{4-[4-(3,3-difluoropiperidin-1-ylcarbonyl)-oxazol-2-yl]-benzyl}-acetamide;
2,2-difluoro-2-(thien-2-yl)-N-{4-[4-(piperidin-1-ylcarbonyl)-oxazol-2-yl]-benzyl}-acetamide;
2,2-difluoro-2-(thien-2-yl)-N-{4-[4-(4-hydroxypiperidin-1-ylcarbonyl)-oxazol-2-yl]-benzyl}-acetamide;
2,2-difluoro-2-phenyl-N-{4-[4-(4-hydroxypiperidin-1-ylcarbonyl)-oxazol-2-yl]-benzyl}-acetamide;
2,2-difluoro-2-phenyl-N-{4-[4-(3-hydroxypiperidin-1-ylcarbonyl)-oxazol-2-yl]-benzyl}-acetamide;
2,2-difluoro-2-phenyl-N-{4-[4-(3,3-difluoropiperidin-1-ylcarbonyl)-oxazol-2-yl]-benzyl}-acetamide;
2-phenyl-N-{4-[4-(trans-4-fluoro-3-hydroxy-(homopiperidin-1-yl)carbonyl)-oxazol-2-yl]-benzyl}-acetamide;
2-(thien-3-yl)-N-{4-[4-(trans-4-fluoro-3-hydroxy-(homopiperidin-1-yl)carbonyl)-oxazol-2-yl]-benzyl}-acetamide;
2-(thien-3-yl)-N-{4-[4-(3-fluoro-4-hydroxypiperidin-1-ylcarbonyl)-oxazol-2-yl]-benzyl}-acetamide;
2,2-difluoro-2-(pyridin-2-yl)-N-{4-[4-(piperidin-1-ylcarbonyl)-oxazol-2-yl]-benzyl}-acetamide;
2,2-difluoro-2-(pyridin-2-yl)-N-{4-[4-(4-hydroxypiperidin-1-ylcarbonyl)-oxazol-2-yl]-benzyl}-acetamide;
2-hydroxy-2-phenyl-N-{4-[4-(piperidin-1-ylcarbonyl)-oxazol-2-yl]-benzyl}-acetamide;
2-hydroxy-2-phenyl-N-{4-[4-(3,3-difluoropiperidin-1-ylcarbonyl)-oxazol-2-yl]-benzyl}-acetamide;
2R-hydroxy-2-phenyl-N-{4-[4-(3,3-difluoropiperidin-1-ylcarbonyl)-oxazol-2-yl]-benzyl}-acetamide;
2-fluoro-2-phenyl-N-{4-[4-(3-chloropiperidin-1-ylcarbonyl)-oxazol-2-yl]-benzyl}-acetamide;
2-(2-fluorophenyl)-N-{4-[4-(3,3-difluoropiperidin-1-ylcarbonyl)-oxazol-2-yl]-benzyl}-acetamide;
2-(3-fluorophenyl)-N-{4-[4-(3,3-difluoropiperidin-1-ylcarbonyl)-oxazol-2-yl]-benzyl}-acetamide;
2-(2-fluorophenyl)-N-{4-[4-(4-hydroxypiperidin-1-ylcarbonyl)-oxazol-2-yl]-benzyl}-acetamide;
2-(3-fluorophenyl)-N-{4-[4-(4-hydroxypiperidin-1-ylcarbonyl)-oxazol-2-yl]-benzyl}-acetamide;
2-(2-fluorophenyl)-N-{4-[4-(3-hydroxypiperidin-1-ylcarbonyl)-oxazol-2-yl]-benzyl}-acetamide;
2-(3-fluorophenyl)-N-{4-[4-(3-hydroxypiperidin-1-ylcarbonyl)-oxazol-2-yl]-benzyl}-30 acetamide;
2-fluoro-2-phenyl-N-{4-[4-(3-fluoropiperidin-1-ylcarbonyl)-oxazol-2-yl]-benzyl}-acetamide;
2S-hydroxy-2-phenyl-N-{4-[4-(3,3-difluoropiperidin-1-ylcarbonyl)-oxazol-2-yl]-benzyl}-acetamide;
2-(thien-3-yl)-N-{4-[4-([1,3]oxazinan-3-ylcarbonyl)-oxazol-2-yl]-benzyl}-acetamide; 2-phenyl-N-{4-[4-([1,3]oxazinan-3-ylcarbonyl)-oxazol-2-yl]-benzyl}-acetamide;
2-fluoro-2-(2-fluorophenyl)-N-{4-[4-(3-hydroxypiperidin-1-ylcarbonyl)-oxazol-2-yl]-benzyl}-acetamide;
2-fluoro-2-(2-fluorophenyl)-N-{4-[4-(4-hydroxypiperidin-1-ylcarbonyl)-oxazol-2-yl]-benzyl}-acetamide;
2-fluoro-2-(2-fluorophenyl)-N-{4-[4-(3,3-difluoropiperidin-1-ylcarbonyl)-oxazol-2-yl]-benzyl}-acetamide;
2-phenyl-N-{4-{4-[3-({[(CH₃)₃C]O(CO)NH}{[(CH₃)₃C]O(CO)CH₂}CH(CO)NH)-4-hydroxypiperidin-1-ylcarbonyl]-oxazol-2-yl}-benzyl}-acetamide;
2-(4-azidophenyl)-N-{4-[4-(piperidin-1-ylcarbonyl)-oxazol-2-yl]-benzyl}-acetamide;
2-(3-azidophenyl)-N-{4-[4-(piperidin-1-ylcarbonyl)-oxazol-2-yl]-benzyl}-acetamide;
2-(4-iodophenyl)-N-{4-[4-(trans-2,5-dimethylpyrrolidin-1-ylcarbonyl)-oxazol-2-yl]-benzyl}-acetamide;
2-(4-iodophenyl)-N-{4-[4-(cis-2,5-dimethylpyrrolidin-1-ylcarbonyl)-oxazol-2-yl]-benzyl}-acetamide;
2-(4-iodophenyl)-N-{4-[4-(trans-2,5-dimethyl-2,5-dihydro-1H-pyrrol-1-ylcarbonyl)-oxazol-2-yl]-benzyl}-acetamide;
2-(4-iodophenyl)-N-{4-[4-(cis-2,5-dimethyl-2,5-dihydro-1H-pyrrol-1-ylcarbonyl)-oxazol-2-yl]-benzyl}-acetamide;
2-phenyl-N-{4-[4-(cis-2,5-dimethyl-2,5-dihydro-1H-pyrrol-1-ylcarbonyl)-oxazol-2-yl]-benzyl}-acetamide;
2-(thien-3-yl)-N-{4-[4-(3-fluoro-4-oxopiperidin-1-ylcarbonyl)-oxazol-2-yl]-benzyl}-acetamide;
2-(2-hydroxyphenyl)-N-{4-[4-(piperidin-1-ylcarbonyl)-oxazol-2-yl]-benzyl}-acetamide;
2-(3-hydroxyphenyl)-N-{4-[4-(piperidin-1-ylcarbonyl)-oxazol-2-yl]-benzyl}-acetamide;
2-(4-hydroxyphenyl)-N-{4-[4-(piperidin-1-ylcarbonyl)-oxazol-2-yl]-benzyl}-acetamide;
2-(3-fluoro-4-hydroxyphenyl)-N-{4-[4-(piperidin-1-ylcarbonyl)-oxazol-2-yl]-benzyl}-acetamide;
2-(3-methylisoxazol-5-yl)-N-{4-[4-(piperidin-1-ylcarbonyl)-oxazol-2-yl]-benzyl}-acetamide;
2-carboxy-2-phenyl-N-{4-[4-(piperidin-1-ylcarbonyl)-oxazol-2-yl]-benzyl}-acetamide;
2-fluoro-2-phenyl-N-{4-{4-[4-(cyclohexylcarbonyloxy)-piperidin-1-ylcarbonyl]-oxazol-2-yl}-benzyl}-acetamide;
2-fluoro-2-phenyl-N-{4-{4-[4-(acetyloxy)piperidin-1-ylcarbonyl]-oxazol-2-yl}-benzyl}-acetamide
2,2-difluoro-2-(thien-3-yl)-N-{4-[4-(3-hydroxypiperidin-1-ylcarbonyl)-oxazol-2-yl]-benzyl}-acetamide
2-amino-2-phenyl-N-{4-[4-(piperidin-1-ylcarbonyl)-oxazol-2-yl]-benzyl}-acetamide;
2-phenyl-N-{4-[4-(3-fluoro-4-hydroxypiperidin-1-ylcarbonyl)-oxazol-2-yl]-benzyl}-acetamide;
2,2-difluoro-2-(thien-2-yl)-N-{4-[4-(3-hydroxy-(homopiperidin-1-yl)carbonyl)-oxazol-2-yl]-benzyl}-acetamide;
2-carboxy-2-phenyl-N-{4-[4-(3,3-difluoropiperidin-1-ylcarbonyl)-oxazol-2-yl]-benzyl}-acetamide;
2,2-difluoro-2-(thien-3-yl)-N-{4-[4-(3-fluoro-4-hydroxypiperidin-1-ylcarbonyl)-oxazol-2-yl]-benzyl}-acetamide;
2-carboxy-2-(thien-3-yl)-N-{4-[4-(3-fluoro-4-hydroxypiperidin-1-ylcarbonyl)-oxazol-2-yl]-benzyl}-acetamide;
2,2-difluoro-2-(thien-2-yl)-N-{4-[4-(4-hydroxy-(homopiperidin-1-yl)carbonyl)-oxazol-2-yl]-benzyl}-acetamide;
3-(4-methoxyphenyl)-N-{4-[4-(4-hydroxypiperidin-1-ylcarbonyl)-oxazol-2-yl]-benzyl}-propionamide;
3-(4-methylphenyl)-N-{4-[4-(4-hydroxypiperidin-1-ylcarbonyl)-oxazol-2-yl]-benzyl}-propionamide;
3-(3,4-difluorophenyl)-N-{4-[4-(4-hydroxypiperidin-1-ylcarbonyl)-oxazol-2-yl]-benzyl}-propionamide;
2-hydroxymethyl-2-phenyl-N-{4-[4-(piperidin-1-ylcarbonyl)-oxazol-2-yl]-benzyl}-acetamide;
2-phenyl-N-{4-[4-(trans-3,4-dihydroxypiperidin-1-ylcarbonyl)-oxazol-2-yl]-benzyl}-acetamide;
2-(thien-3-yl)-N-{4-[4-(trans-3,4-dihydroxypiperidin-1-ylcarbonyl)-oxazol-2-yl]-benzyl}-acetamide;
2-(thien-2-yl)-N-{4-[4-(trans-3,4-dihydroxypiperidin-1-ylcarbonyl)-oxazol-2-yl]-benzyl}-acetamide;
2-(4-azidophenyl)-N-{4-[4-(trans-3,4-dihydroxypiperidin-1-ylcarbonyl)-oxazol-2-yl]-benzyl}-acetamide;
2-(3-azidophenyl)-N-{4-[4-(trans-3,4-dihydroxypiperidin-1-ylcarbonyl)-oxazol-2-yl]-benzyl}-acetamide;
2-phenyl-N-{4-[4-(3-aza-bicyclo[3.1.0]hexan-3-ylcarbonyl)-oxazol-2-yl]-benzyl}-acetamide;
2-phenyl-N-{4-[4-(3-oxopiperazin-1-ylcarbonyl)-oxazol-2-yl]-benzyl}-acetamide; 2-phenyl-N-{4-[4-([1,4]oxazepan-4-ylcarbonyl)-oxazol-2-yl]-benzyl}-acetamide;
2-ethoxycarbonyl-2-phenyl-N-{4-[4-(piperidin-1-ylcarbonyl)-oxazol-2-yl]-benzyl}-acetamide;
2-(N-methylaminocarbonyl)-2-phenyl-N-{4-[4-(piperidin-1-ylcarbonyl)-oxazol-2-yl]-benzyl}-acetamide;
2-(N,N-dimethylaminocarbonyl)-2-phenyl-N-{4-[4-(piperidin-1-ylcarbonyl)-oxazol-2-yl]-benzyl}-acetamide;
2-phenyl-N-{4-[4-(2-trifluoromethylpyrrolidin-1-ylcarbonyl)-oxazol-2-yl]-benzyl}-acetamide;
2-phenyl-N-{4-[4-(4-trifluoromethylpiperidin-1-ylcarbonyl)-oxazol-2-yl]-benzyl}-acetamide;
2-(thien-3-yl)-N-{4-[4-(3-oxopiperazin-1-ylcarbonyl)-oxazol-2-yl]-benzyl}-acetamide;
2-(thien-3-yl)-N-{4-[4-([1,4]oxazepan-4-ylcarbonyl)-oxazol-2-yl]-benzyl}-acetamide;
2-(thien-3-yl)-N-{4-[4-(2-trifluoromethylpyrrolidin-1-ylcarbonyl)-oxazol-2-yl]-benzyl}-acetamide;
2-(thien-3-yl)-N-{4-[4-(4-trifluoromethylpiperidin-1-ylcarbonyl)-oxazol-2-yl]-benzyl}-acetamide;
2-(thien-3-yl)-N-{4-[4-(3-aza-bicyclo[3.1.0]hexan-3-ylcarbonyl)-oxazol-2-yl]-benzyl}-acetamide;
2-phenyl-N-{4-[4-(cis-3-hydroxy-4-hydroxymethylpiperidin-1-ylcarbonyl)-oxazol-2-yl]-benzyl}-acetamide;
2-phenyl-N-{4-[4-(trans-3-hydroxy-4-hydroxymethylpiperidin-1-ylcarbonyl)-oxazol-2-yl]-20 benzyl}-acetamide;
2-phenyl-N-{4-[4-(cis-4-hydroxy-3-hydroxymethylpiperidin-1-ylcarbonyl)-oxazol-2-yl]-benzyl}-acetamide;
2-(thien-3-yl)-N-{4-[4-(cis-4-hydroxymethyl-3-hydroxypiperidin-1-ylcarbonyl)-oxazol-2-yl]-benzyl}-acetamide;
2-(thien-3-yl)-N-{4-[4-(trans-4-hydroxymethyl-3-hydroxypiperidin-1-ylcarbonyl)-oxazol-2-yl]-benzyl}-acetamide;
2-(thien-3-yl)-N-{4-[4-(cis-4-hydroxy-3-hydroxymethylpiperidin-1-ylcarbonyl)-oxazol-2-yl]-benzyl}-acetamide.
2-(thien-3-yl)-N-{4-[4-(piperidin-1-ylcarbonyl)-oxazol-2-yl]-(3,5-difluorobenzyl)}-acetamide;
2-(thien-3-yl)-N-{4-[4-(piperidin-1-ylcarbonyl)-oxazol-2-yl]-(3-methylbenzyl)}-acetamide;
2-phenyl-N-{4-[4-(piperidin-1-ylcarbonyl)-oxazol-2-yl]-(3,5-difluorobenzyl)}-acetamide;
2-(4-trifluoromethoxyphenyl)-N-{4-[4-(piperidin-1-ylcarbonyl)-oxazol-2-yl]-(3,5-difluorobenzyl)}-acetamide;
2-phenyl-N-{4-[4-(piperidin-1-ylcarbonyl)-oxazol-2-yl]-(3-methylbenzyl)}-acetamide;
2-(4-trifluoromethoxyphenyl)-N-{4-[4-(piperidin-1-ylcarbonyl)-oxazol-2-yl]-(3-methylbenzyl)}-acetamide;
2-(4-methoxyphenyl)-N-{4-[4-(piperidin-1-ylcarbonyl)-oxazol-2-yl]-(3-methylbenzyl)}-acetamide;
2-(thien-2-yl)-N-{4-[4-(piperidin-1-ylcarbonyl)-oxazol-2-yl]-(3-methylbenzyl)}-acetamide;
2-phenyl-N-{4-[4-(piperidin-1-ylcarbonyl)-oxazol-2-yl]-(2-methoxybenzyl)}-acetamide;
2-(thien-2-yl)-N-{4-[4-(piperidin-1-ylcarbonyl)-oxazol-2-yl]-(2-methoxybenzyl)}-acetamide;
2-fluoro-2-phenyl-N-{4-[4-(piperidin-1-ylcarbonyl)-oxazol-2-yl]-(3-methylbenzyl)}-acetamide;
2S-hydroxy-2-phenyl-N-{4-[4-(piperidin-1-ylcarbonyl)-oxazol-2-yl]-(3-methylbenzyl)}-acetamide;
2R-hydroxy-2-phenyl-N-{4-[4-(piperidin-1-ylcarbonyl)-oxazol-2-yl]-(3-methylbenzyl)}-acetamide;
2S-amino-2-phenyl-N-{4-[4-(piperidin-1-ylcarbonyl)-oxazol-2-yl]-(3-methylbenzyl)}-acetamide
2-phenyl-N-{4-[4-(piperidin-1-ylcarbonyl)-oxazol-2-yl]-(2-hydroxybenzyl)}-acetamide;
2-phenyl-N-{4-[4-(piperidin-1-ylcarbonyl)-oxazol-2-yl]-(3-hydroxybenzyl)}-acetamide;
2-fluoro-2-phenyl-N-{4-[4-(4-hydroxypiperidin-1-ylcarbonyl)-oxazol-2-yl]-2-iodobenzyl}-acetamide;
2-phenyl-N-{4-[4-(3,3-difluoropiperidin-1-ylcarbonyl)-oxazol-2-yl]-2-nitrobenzyl}-acetamide;
2-phenyl-N-{4-[4-(3,3-difluoropiperidin-1-ylcarbonyl)-oxazol-2-yl]-2-iodobenzyl}-acetamide;
2-phenyl-N-{4-[4-(3,3-difluoropiperidin-1-ylcarbonyl)-oxazol-2-yl]-2-(methoxycarbonylethylen-1-yl)benzyl}-acetamide;
2-phenyl-N-{4-[4-(3,3-difluoropiperidin-1-ylcarbonyl)-oxazol-2-yl]-2-(methoxycarbonylethyl)benzyl}-acetamide;
2-phenyl-N-{4-[4-(3,3-difluoropiperidin-1-ylcarbonyl)-oxazol-2-yl]-2-(carboxyethylen-1-yl)benzyl}-acetamide;
2-phenyl-N-{4-[4-(3,3-difluoropiperidin-1-ylcarbonyl)-oxazol-2-yl]-2-aminobenzyl}-acetamide;
2-phenyl-N-{4-[4-(3,3-difluoropiperidin-1-ylcarbonyl)-oxazol-2-yl]-2-acetylaminobenzyl}-acetamide;
2-phenyl-N-{4-[4-(4-hydroxpiperidin-1-ylcarbonyl)-oxazol-2-yl]-2-carboxyethylbenzyl}-acetamide;
2,2-difluoro-2-(thien-2-yl)-N-{4-[4-(3,3-difluoropiperidin-1-ylcarbonyl)-oxazol-2-yl]-2-iodobenzyl}-acetamide;
2,2-difluoro-2-(thien-2-yl)-N-{4-[4-(3,3-difluoropiperidin-1-ylcarbonyl)-oxazol-2-yl]-2-(methoxycarbonylethylen-1-yl)benzyl}-acetamide;
2-phenyl-N-{4-[4-(3,3-difluoropiperidin-1-ylcarbonyl)-oxazol-2-yl]-2-(2-carboxyethylcarbonylamino)benzyl}-acetamide;
2,2-difluoro-2-(thien-2-yl)-N-{4-[4-(3,3-difluoropiperidin-1-ylcarbonyl)-oxazol-2-yl]-2-methoxycarbonylethylbenzyl}-acetamide;
2,2-difluoro-2-(thien-2-yl)-N-{4-[4-(3,3-difluoropiperidin-1-ylcarbonyl)-oxazol-2-yl]-2-carboxyethylbenzyl}-acetamide;
2,2-difluoro-2-(thien-2-yl)-N-{4-[4-(3,3-difluoropiperidin-1-ylcarbonyl)-oxazol-2-yl]-2-methoxycarbonylbenzyl}-acetamide;
2-(thien-3-yl)-N-{4-[4-(4-hydroxypiperidin-1-ylcarbonyl)-oxazol-2-yl]-3,5-difluorobenzyl}-acetamide;
2-phenyl-N-{4-[4-(piperidin-1-ylcarbonyl)-oxazol-2-yl]-3-methoxybenzyl}-acetamide;
2,2-difluoro-2-(thien-2-yl)-N-{4-[4-(3,3-difluoropiperidin-1-ylcarbonyl)-oxazol-2-yl]-2-carboxybenzyl}-acetamide;
2-phenyl-N-{4-[4-(3,3-difluoropiperidin-1-ylcarbonyl)-oxazol-2-yl]-2-(piperazin-1-ylcarbonylethyl)benzyl}-acetamide;
2-phenyl-N-{4-[4-(4-hydroxypiperidin-1-ylcarbonyl)-oxazol-2-yl]-2-(morpholin-4-ylcarbonylethyl)benzyl}-acetamide;
2-phenyl-N-{4-[4-(piperidin-1-ylcarbonyl)-oxazol-2-yl]-3-methoxycarbonylmethyloxy-benzyl}-acetamide;
2-phenyl-N-{4-[4-(4-hydroxypiperidin-1-ylcarbonyl)-oxazol-2-yl]-2-methoxycarbonylbenzyl}-acetamide.
2-phenyl-N-{4-[5-(3,3-difluoropiperidin-1-yl-carbonyl)-1H-imidazol-2-yl]-benzyl}-acetamide; and
2-phenyl-N-{4-[4-(2,5-dimethylpiperidin-1-yl-carbonyl)-1H-imidazol-2-yl]-benzyl}-acetamide.
2-phenyl-N-{4-[5-(piperidin-1-yl-carbonyl)-[1,2,4]oxadiazol-3-yl]-benzyl}-acetamide.
2-phenyl-N-{4-[4-(piperidin-1-ylcarbonyl)-thiazol-2-yl]-benzyl}-acetamide;
2-(thien-2-yl)-N-{4-[4-(piperidin-1-ylcarbonyl)-thiazol-2-yl]-benzyl}-acetamide;
1-phenyl-N-{4-[4-(piperidin-1-ylcarbonyl)-thiazol-2-yl]-benzyl}-methanesulfonamide;
3-phenyl-N-{4-[4-(piperidin-1-ylcarbonyl)-thiazol-2-yl]-benzyl}-propionamide.
2-(thien-3-yl)-N-{4-[4-(3,3-difluoropiperidin-1-ylcarbonyl)-pyrazol-1-yl]-benzyl}-acetamide;
2-(thien-3-yl)-N-{4-[4-(piperidin-1-ylcarbonyl)-pyrazol-1-yl]-benzyl}-acetamide;
2-phenyl-N-{4-[4-(piperidin-1-ylcarbonyl)-pyrazol-1-yl]-benzyl}-acetamide;
2-phenyl-N-{4-[4-(3-hydroxypiperidin-1-ylcarbonyl)-pyrazol-1-yl]-benzyl}-acetamide;
2-phenyl-N-{4-[4-(4-hydroxypiperidin-1-ylcarbonyl)-pyrazol-1-yl]-benzyl}-acetamide;
2-phenyl-N-{4-[4-(3,3-difluoropiperidin-1-ylcarbonyl)-pyrazol-1-yl]-benzyl}-acetamide;
2-(thien-3-yl)-N-{4-[4-(3-hydroxypiperidin-1-ylcarbonyl)-pyrazol-1-yl]-benzyl}-acetamide;
2-(thien-3-yl)-N-{4-[4-(4-hydroxypiperidin-1-ylcarbonyl)-pyrazol-1-yl]-benzyl}-acetamide; or
a pharmaceutically acceptable salt thereof.

27. A compound selected from the group consisting of:
2-phenyl-N-{4-[4-(3,3-difluoropiperidin-1-ylcarbonyl)-oxazol-2-yl]-benzyl}-acetamide;
2-(4-trifluoromethoxyphenyl-N-{4-[4-(piperidin-1-ylcarbonyl)-oxazol-2-yl]-benzyl}-acetamide;
2-fluoro-2-phenyl-N-{4-[4-(4-hydroxypiperidin-1-ylcarbonyl)-oxazol-2-yl]-benzyl}-acetamide;
2-(thien-3-yl)-N-{4-[4-(piperidin-1-ylcarbonyl)-oxazol-2-yl]-3,5-difluorobenzyl}-acetamide;
2-phenyl-N-{4-[4-(3-hydroxypiperidin-1-ylcarbonyl)-oxazol-2-yl]-benzyl}-acetamide;
2-(4-chlorophenyl)-N-{4-[4-(piperidin-1-ylcarbonyl)-oxazol-2-yl]-benzyl}-acetamide;
2-(thien-3-yl)-N-{4-[4-(3-hydroxypiperidin-1-ylcarbonyl)-oxazol-2-yl]-benzyl}-acetamide;
2-(thien-3-yl)-N-{4-[4-(3,3-difluoropiperdin-1-ylcarbonyl)-oxazol-2-yl]-benzyl}-25 acetamide;
2-(thien-3-yl)-N-{4-[4-(piperidin-1-ylcarbonyl)-oxazol-2-yl]-3-methylbenzyl}-acetamide;
2-phenyl-N-{4-[4-(piperidin-1-ylcarbonyl)-thiazol-2-yl]-benzyl}-acetamide;
2-(thien-3-yl)-N-{4-[4-(homopiperidin-1-ylcarbonyl)-oxazol-2-yl]-benzyl}-acetamide;
2-phenyl-N-{4-[4-(piperidin-1-ylcarbonyl)-oxazol-2-yl]-benzyl}-acetamide;
2-fluoro-2-phenyl-N-{4-[4-(piperidin-1-ylcarbonyl)-oxazol-2-yl]-benzyl}-acetamide;
2-(thien-3-yl)-N-{4-[4-(piperidin-1-ylcarbonyl)-oxazol-2-yl]-benzyl}-acetamide;
2,2-difluoro-2-(thien-3-yl)-N-{4-[4-(piperidin-1-ylcarbonyl)-oxazol-2-yl]-benzyl}-acetamide;
2-phenyl-N-{4-[4-(homopiperidin-1-ylcarbonyl)-oxazol-2-yl]-benzyl}-acetamide;
2,2-difluoro-2-(thien-3-yl)-N-{4-[4-(4-hydroxyhomopiperidin-1-ylcarbonyl)-oxazol-2-yl]-benzyl}-acetamide;
2,2-difluoro-2-(thien-3-yl)-N-{4-[4-(3-hydroxypiperidin-1-ylcarbonyl)-oxazol-2-yl]-benzyl}-acetamide;
2-(thien-3-yl)-N-{4-[4-(4-hydroxyhomopiperidin-1-ylcarbonyl)-oxazol-2-yl]-benzyl}-acetamide;
2-(thien-3-yl)-N-{4-[4-(3,3-difluoro-4-hydroxypiperidin-1-ylcarbonyl)-oxazol-2-yl]-benzyl}-acetamide; and 2-fluoro-2-(2-fluorophenyl)-N-{4-[4-(piperidin-1-ylcarbonyl)-oxazol-2-yl]-benzyl}-acetamide;
2-(thien-3-yl)-N-{4-[4-(3-hydroxypiperidin-1-ylcarbonyl)-oxazol-2-yl]-3,5-difluorobenzyl}-acetamide;
2-(thien-3-yl)-N-{4-[4-(4-oxopiperidin-1-ylcarbonyl)-oxazol-2-yl]-3,5-difluorobenzyl}-acetamide;
2-(thien-3-yl)-N-{4-[4-(trans-3,4-dihydroxypiperidin-1-ylcarbonyl)-oxazol-2-yl]-3,5-difluorobenzyl}-acetamide
2-phenyl-N-{4-[4-(8-oxa-3-aza-bicyclo[4.2.0]octan-3-ylcarbonyl)-oxazol-2-yl]-benzyl}-acetamide;
2-phenyl-N-{4-[4-(7-oxa-3-aza-bicyclo[4.2.0]octan-3-ylcarbonyl)-oxazol-2-yl]-benzyl}-acetamide;
2-phenyl-N-{4-[4-(4-acetylhomopiperazin-1-ylcarbonyl)-oxazol-2-yl]-benzyl}-acetamide
2-(thien-3-yl)-N-{4-[4-(8-oxa-3-aza-bicyclo[4.2.0] octan-3-ylcarbonyl)-oxazol-2-yl]-benzyl}-acetamide;
2-(thien-3-yl)-N-{4-[4-(7-oxa-3-aza-bicyclo [4.2.0]octan-3-ylcarbonyl)-oxazol-2-yl]-benzyl}-acetamide;
2-(thien-3-yl)-N-{4-[4-(4-acetylhomopiperazin-1-ylcarbonyl)-oxazol-2-yl]-benzyl}-acetamide
2-(thien-3-yl)-N-{4-[4-(pyrrolidin-1-ylcarbonyl)-oxazol-2-yl]-3,5-difluorobenzyl}-acetamide; or
a pharmaceutically acceptable salt thereof.

28. A method of treating a disorder responsive to the induction of apoptosis in an animal suffering said disorder, comprising administering to said animal a pharmaceutical composition comprising a therapeutically effective amount of a compound of Formula II: 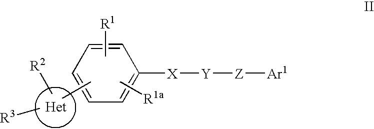
wherein:
R¹ and R a are independently hydrogen, alkyl, alkoxy, halo, haloalkyl, nitro, amino, alkylamino, dialkylamino, alkylcarbonylamino, carboxy, alkoxycarbonyl, carboxyalkyl, alkoxycarbonylalkenyl, hydroxy, alkoxycarbonylalkyloxy, alkoxycarbonylalkyl, carboxyalkylcarbonylamino, carboxyalkenyl, saturated or unsaturated heterocycloalkylaminocarbonylalkyl, or hydroxyalkyl; or when R¹ and R^{1a} are adjacent to each other they may combine to form a CH═CH—CH═CH— group;
R² is hydrogen, alkyl, hydroxyalkyl, aryl, heteroaryl, or halo;
R³ is —CONR⁴R⁵ where R⁴ and R⁵ together with the nitrogen atom to which they are attached form saturated or unsaturated heterocycloalkylamino, saturated or unsaturated bicyclic heterocycloalkylamino, or bridged or unbridged heterocycloalkylamino;
Het is a five membered heteroaryl ring consisting of one, two, three, or four heteroatoms independently selected from nitrogen, oxygen, or sulfur, the remaining ring atoms being carbon;
X is alkylene optionally substituted with halo;
Y is —O—, —NR⁶—, —S—, —SO—, —SO₂—, —NR⁷CO—, —CONR⁷—, —NR⁷SO₂—, —SO₂NR⁷—, —NHCONH—, —NHCSNH—, —NHCOO—, or —OCONH— where R⁶ and R⁷ are independently hydrogen or alkyl;
Z is alkenylene or alkylene wherein said alkylene is optionally substituted with halo, hydroxy, hydroxyalkyl, carboxy, amino, amido, alkoxycarbonyl, alkylaminocarbonyl, or dialkylaminocarbonyl; and
Ar¹ is aryl, heteroaryl, or saturated or unsaturated heterocycloalkyl; or a pharmaceutically acceptable salt thereof and a pharmaceutically acceptable excipient.

29. The method of claim 28 wherein the disease is a cancer.

30. A method of treating cancer in an animal which method comprises administering to said animal a pharmaceutical composition comprising a therapeutically effective amount of a compound of Formula I or II and a pharmaceutically acceptable excipient in combination with radiation therapy and optionally in combination with one or more chemotherapeutic compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.

31. The method of claim 24 wherein the chemotherapeutic compound(s) is independently selected from Taxol®, Taxotere®, epothilone A, epothilone B, desoxyepothilone A, desoxyepothilone B or their derivatives; epidophyllotoxin; procarbazine; mitoxantrone; the mitomycins, discodermolide, podophyllotoxins, doxorubicin, carminomycin, daunorubicin, aminopterin, methotrexate, methopterin, dichloro-methotrexate, mitomycin C, porfiromycin, Herceptin®, Rituxan®, 5-fluorouracil, 6-mercaptopurine, gemcitabine, cytosine arabinoside, colchicines, etoposide, etoposide phosphate or teniposide, melphalan, vinblastine, vincristine, leurosidine, vindesine, leurosine, paclitaxel, estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

32. A pharmaceutical composition comprising a therapeutically effective amount of a compound of Formula I or II and a pharmaceutically acceptable excipient.

33. A process of preparing a compound of Formula I or II where Y is —NR⁷CO—comprising:
(a) reacting a compound of Formula III: 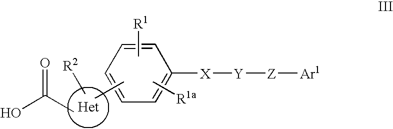
where R¹, R^{1a} R², X, Z, and Ar¹ are as defined for a compound of Formula I above and Y is —NR⁷CO— where R⁷ is as defined for a compound of Formula I above; with an amine of formula NHR⁴R⁵ where R⁴ and R⁵ together with the nitrogen atom to which they are attached form saturated or unsaturated heterocycloalkylamino, saturated or unsaturated bicyclic heterocycloalkylamino, or bridged saturated or unsaturated heterocycloalkylamino to provide a compound of Formula I or II; or
(b) reacting a compound of Formula IV: 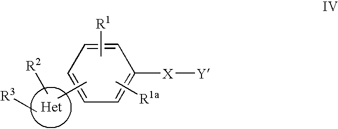
where R¹, R^{1a}, R², R³, X, are as defined for a compound of Formula I above and Y′ is —NHR⁷ where R⁷ is as defined for a compound of Formula I above, with an acylating agent of formula Ar¹—Z—CO₂H or Ar¹—Z—COLG where LG is a leaving group under acylating reaction conditions to provide a compound of Formula I or II, where Y is —NR⁷CO—;
(c) optionally converting the compound obtained in step (a) or (b) above, to an acid addition salt;
(d) optionally converting a salt form of the compound obtained in step (a) or (b) above, to a free base;
(e) optionally separating individual isomers;
(f) optionally modifying any of the R¹, R^{1a} R², R³, and Ar¹ groups.
